(19) United States
(12) Patent Application Publication
(10) Pub. No.: US 2024/0024253 A1
(43) Pub. Date: Jan. 25, 2024

(54) Continuous Drug Delivery Systems and Methods

(71) Applicant: Pike Therapeutics Inc., Vancouver (CA)

(72) Inventors: Brad MILES, Vancouver (CA); Fotios M. PLAKOGIANNIS, Whitestone, NY (US)

(73) Assignee: Pike Therapeutics Inc., Vancouver, BC (CA)

(21) Appl. No.: 18/224,183

(22) Filed: Jul. 20, 2023

Related U.S. Application Data
(60) Provisional application No. 63/391,004, filed on Jul. 21, 2022.

(51) Int. Cl. A61K 9/70; A61K 31/00; A61K 9/00

(52) U.S. Cl. CPC A61K 9/7038; A61K 31/658; A61K 9/0014

(57) ABSTRACT

Compositions and methods for the treatment and/or prevention and/or control of indications such as pain management, cancer, seizure disorders, and Parkinson's Disease using continuous drug delivery.

### CROSS-REFERENCE TO RELATED APPLICATION

This application claims the benefit under 35 U.S.C. § 119(e) of Application Ser. No. 63/391,004 filed on Jul. 21, 2022, entitled CONTINUOUS DRUG DELIVERY SYSTEMS AND METHODS and whose entire disclosure is incorporated by reference herein

### BACKGROUND

The disclosure provides systems, compositions, and methods for the treatment and/or prevention and/or control of indications such as pain management, cancer, seizure disorders, and Parkinson's Disease using continuous drug delivery. The continuous drug delivery methods and systems as disclosed herein may provide active agent in several different ways, such as through infusion therapy via, for example, intra or subcutaneous application, or transdermal or topical application. Infusion therapy administers medication through the use of a sterile thin tube such as catheter that is inserted into the body and secured. A pump delivery system and any other delivery system that would deliver a continuous infusion such as depot injection and ambulatory pumps can be used for this purpose. Continuous drug delivery systems and methods also may include, for example, transdermal systems. In Transdermal drug delivery, a transdermal patch or transdermal composition is applied topically to the skin surface. Throughout the duration of topical application of a transdermal patch or transdermal composition drug is continuously released and delivered through the intact skin (via transcellular, intercellular and transappendageal routes) to achieve systemic effect. Therefore, once applied transdermal composition or transdermal patch can deliver drug into systemic circulation throughout the day or even for more than one day depending on the duration of its application which can be even up to a week.

In continuous drug delivery, active agent is continuously released and delivered to the patient to achieve systemic effect. Therefore, once applied a continuous drug delivery system can deliver active agents into systemic circulation throughout a day or even for more than one day depending on the duration of its application which can be even up to a week.

Through continuous drug delivery, for example, purified tetrahydrocannabinol (THC), cannabidiol (CBD), or combinations thereof, can be delivered throughout the duration of application. Depending on the requirement, the duration of application can be once in a day, once in two days, once in three days, once in four days, once in five days, once in a week. Therefore, continuous drug delivery can overcome the multiple dose regimen of oral delivery by reducing the dosing frequency.

In continuous drug delivery the pharmaceutical composition comprising active agent is delivered slowly and continuously throughout the duration of administration, hence there are no peaks and troughs in drug plasma concentration of the active agent which are associated with multiple dose administration, such as oral administration, in a day. Therefore, by continuous drug delivery of, for example, tetrahydrocannabinol (THC), cannabidiol (CBD), or combinations thereof, patients can have the therapeutic effect of the drug for extended period of time without drastic changes in drug plasma concentration.

In continuous drug delivery active using the systems and methods as disclosed herein active agent is delivered into systemic circulation and escapes the first pass hepatic metabolism, therefore to achieve the desired therapeutic activity less drug is required, resulting into less adverse effects or side effects. Tetrahydrocannabinol (THC) and Cannabidiol (CBD) have high lipid solubility and after oral administration undergoes hepatic first pass metabolism, therefore of the administered dose only 10%-20% reaches systemic circulation, thus as compared to oral dose, a continuous drug delivery a small dose of tetrahydrocannabinol (THC), cannabidiol (CBD), or combinations thereof can give the desired therapeutic effects at a lower dose than oral.

Furthermore, using a continuous drug delivery system and methods as disclosed herein is easy, noninvasive and convenient. Administration of a drug compound by continuous drug does not require medical supervision as patients can control the administration themselves.

In certain embodiments, the continuous drug delivery system and methods as disclosed herein may comprise a pump device, such as ones which are commonly used to deliver one or more fluids to a targeted individual. For example, a medical infusion pump device may be used to deliver a medicine to a patient as part of a medical treatment. The active agent(s) that is delivered by the infusion pump device can depend on the condition of the patient and the desired treatment plan. For example, infusion pump devices have been used to deliver insulin into the subcutaneous tissue and to the vasculature of diabetes patients to regulate blood-glucose levels. In some circumstances, the dosage of medicine delivered by the infusion pump can be calculated by the infusion pump system. In these circumstances, the infusion pump system can take into account many variables, including user input, when making such calculations.

Other forms of continuous drug delivery systems and methods may make use of fluid delivery to a targeted individual. For example, insulin, glucagon, or another medicine can be injected using a manual syringe or a single use injection “pen.” In some circumstances, an injectable form of glucagon is used in emergency aid of severe hypoglycemia when the victim is unconscious or for other reasons cannot take glucose orally. The glucagon fluid can be rapidly injected to the patient by intramuscular, intravenous, or subcutaneous injection, and quickly raises the blood glucose level of the patient.

One category of devices for delivering such fluids is that of pumps that have been developed for the administration of insulin and other medicaments for those suffering from both type I and type II diabetes. Some pumps configured as portable infusion devices can provide Continuous drug for the treatment of diabetes. Such therapy may include, e.g., the regular and/or continuous injection or infusion of insulin into the skin of a person suffering from diabetes and offer an alternative to multiple daily injections of insulin by an insulin syringe or an insulin pen. Such pumps can be ambulatory/portable infusion pumps that are worn by the user and may use replaceable cartridges. Examples of such pumps and various features that can be associated with such pumps include those disclosed in U.S. patent application Ser. No. 13/557,163, U.S. patent application Ser. No. 12/714,299, U.S. patent application Ser. No. 12/538,018, U.S. patent application Ser. No. 13/838,617, U.S. patent application Ser. No. 13/827,707 and U.S. Pat. No. 8,287,495, each of which is hereby incorporated herein by reference in its entirety.

In certain embodiments as disclosed herein continuous drug delivery system may comprise, for example, a patch pump, or micro pump. Patch pumps are small pumps, typically ambulatory, that are carried directly on the skin under the user's clothing. Such a pump generally is situated directly on the injection site such that no tubing is required to deliver the insulin or other medicament to the patient. Patch pumps typically are at least in part disposable, meant to be worn for a day or two and then discarded for a new patch pump.

Cannabis (marijuana) is a schedule-I drug in USA. Cannabis is a flowering plant which contains more than 400 phytonutrient (micronutrient). More than 100 different types of terpenoids, essential oils, antioxidants and cannabinoids have been extracted from the plant. From all of the phytochemicals, only tetrahydrocannabinol (THC) showed significant psychoactive effect. A number of research papers have been published on THC due to its psychoactive and therapeutic effects. Apart from THC, several other constituents have been studied, which also showed some therapeutic effect without psychoactive effect such as cannabidiol (CBD), cannabinol (CBN), cannabichromene (CBC), cannabigerol (CBG), tetrahydrocannabivarin (THCV), delta 9-tetrahydrocannabinol (delta9THC) and many more. It has been showed that cannabis and its derivatives can be used for the treatment of pain, type-2 related metabolic disorder, decrease intraocular pressure, Dravet syndrome, Lennox-Gastaut Syndrome (LGS), epilepsy, nausea, pain and wasting associated with AIDS, arthritis and rheumatism, migraines, muscle spasticity associated with multiple sclerosis and paralysis, alcohol and narcotics withdrawal, stress and depression, asthma, fibromyalgia, inflammatory pain, and pain and/or inflammation associated with chemotherapy, act as an antimicrobial. FDA approved Marinol and Syndros contains delta 9-THC, which currently used in treatment of nausea, vomiting, and anorexia associated with chemotherapy treatments. In clinical studies Sativex (cannabinoid extract oromucosal spray containing THC and CBD) has shown improvements in neuropathic pain and sleep quality. Furthermore, in April 2016 FDA gave orphan drug designation to cannabidiol for the treatment of Tuberous Sclerosis Complex (TSC), Dravet Syndrome and Lennox-Gastaut Syndrome. Cannabidiol is an orally effective treatment for pain and inflammation (Costa, B. The non-psychoactive cannabis constituent cannabidiol is an orally effective therapeutic agent in rat chronic inflammatory and neuropathic pain. European Journal of Pharmacology. Volume 556, Issues 1-3, 5 Feb. 2007, Pages 75-83).

With respect to tetrahydrocannabinol (THC), cannabidiol (CBD), or combinations thereof it is expected that interpatient variability in pharmacologic response will be less with continuous drug delivery as drug plasma concentration can be controlled by controlling the rate of drug delivery. With Continuous drug delivery a small amount of tetrahydrocannabinol (THC), cannabidiol (CBD), or combinations thereof can be delivered for longer duration than oral administration. Continuous drug formulations of tetrahydrocannabinol (THC), cannabidiol (CBD), or combinations thereof can also provide more abuse deterrence than immediate release dosage forms.

Moreover, in case of any adverse effect, side effect or emergency continuous drug delivery gives the liberty to terminate the therapy anytime by taking off the continuous drug delivery system.

There is a need for an improved drug delivery system of CBD and/or THC which can overcome the drawbacks associated with oral routes. Continuous drug delivery of for example, CBD and/or THC, the free base thereof, salts thereof, isomers thereof, amorphous forms thereof, crystalline forms thereof, co crystalline forms thereof, prodrugs thereof, analogs thereof, derivatives thereof, synthetic forms thereof, biosynthetic forms thereof, active metabolites thereof, solid solution thereof, polymorphs thereof, stereoisomers thereof, coated form thereof, ion-pairs thereof, solution thereof in solvents alone or in combinations thereof can address the challenges associated with oral drug delivery, and are useful as treatment, prevention and/or control of, for example, indications such as pain management, cancer, seizure disorders, Parkinson's Disease, the symptoms associated with PD, and other indications.

All references cited herein are incorporated herein by reference in their entireties.

### BRIEF SUMMARY

The disclosure provides a method of treating, preventing, and/or slowing the onset or progression of a disease or condition and/or related symptom in a patient comprising: selecting a patient in need of treating, preventing, and/or slowing the onset or progression of a disease or condition and/or a related symptom; administering to the patient a pharmaceutical composition in a continuous delivery system, the pharmaceutical composition comprising at least one active agent selected from the group consisting of: about 0.1% to about 50% of an active agent selected from the group consisting of cannabidiol (CBD), synthetic forms thereof, biosynthetic forms thereof, free base forms thereof, salts thereof, isomers thereof, amorphous forms thereof, derivatives thereof, and combinations thereof; about 0.1% to about 50% of an active agent selected from the group consisting of tetrahydrocannabinol (THC), synthetic forms thereof, biosynthetic forms thereof, free base forms thereof, salts thereof, isomers thereof, amorphous forms thereof, derivatives thereof, and combinations thereof; optionally, at least one additional active agent, and combinations thereof, wherein the disease or condition and/or related symptom is treated, prevented, and/or slowed in the patient. The disclosure provides a method of treating, preventing, and/or slowing the onset or progression of a disease or condition and/or related symptom in a patient wherein the continuous delivery system provides a continuous, sustained delivery of the pharmaceutical composition to mitigate peak and valley pharmacokinetic behavior of the active agent. The disclosure provides a method of treating, preventing, and/or slowing the onset or progression of a disease or condition and/or related symptom in a patient wherein the continuous delivery system provides a continuous, sustained delivery of the pharmaceutical composition via administration to the patient by a route selected from the group consisting of parenteral, intravenous, subcutaneous, intramuscular, intrathecal, oral, buccal, mucosal, intranasal, rectal, vaginal, transdermal, implantable, topical, and combinations thereof. The disclosure provides a method of treating, preventing, and/or slowing the onset or progression of a disease or condition and/or related symptom in a patient wherein the continuous delivery system provides a continuous, sustained delivery of the pharmaceutical composition to the patient via intravenous or subcutaneous infusion. The disclosure provides a method of treating, preventing, and/or slowing the onset or progression of a disease or condition and/or related symptom in a patient wherein the continuous delivery system provides a continuous, sustained delivery of the pharmaceutical composition to the patient via subcutaneous infusion. The disclosure provides a method of treating, preventing, and/or slowing the onset or progression of a disease or condition and/or related symptom in a patient wherein the continuous delivery system provides a continuous, sustained delivery of the pharmaceutical composition to the patient via an infusion pump device.

The disclosure provides a method of treating, preventing, and/or slowing the onset or progression of a disease or condition and/or related symptom in a patient wherein the continuous delivery system provides a continuous, sustained delivery of the pharmaceutical composition to the patient via an ambulatory/portable infusion pump that is worn by the patient and may use replaceable cartridges. The disclosure provides a method of treating, preventing, and/or slowing the onset or progression of a disease or condition and/or related symptom in a patient wherein the continuous delivery system provides a continuous, sustained delivery of the pharmaceutical composition to the patient via a patch pump or micro pump. The disclosure provides a method of treating, preventing, and/or slowing the onset or progression of a disease or condition and/or related symptom in a patient wherein the continuous delivery system comprises a pharmaceutical composition selected from the group consisting of a liquid formulation, a solid formulation, a semi-solid formulation, an emulsion formulation, a nanoparticle formulation, a matrix formulation, a film formulation, a patch formulation, a formulation in an infusion pump, and/or combinations thereof. The disclosure provides a method of treating, preventing, and/or slowing the onset or progression of a disease or condition and/or related symptom in a patient wherein the continuous delivery system comprises a pharmaceutical composition selected from the group consisting of transdermal liquid formulation, transdermal semisolid formulation, transdermal gel formulation, or transdermal polymer matrix formulation, transdermal adhesive matrix formulation, transdermal film forming gel, transdermal film forming spray formulation, multilayer transdermal matrix system, transdermal drug-in-adhesive matrix formulation, and combinations thereof. The disclosure provides a method of treating, preventing, and/or slowing the onset or progression of a disease or condition and/or related symptom in a patient wherein the continuous delivery system comprises a pharmaceutical composition formulated as a topical liquid formulation, topical semisolid formulation, topical gel formulation, topical polymer matrix formulation, topical adhesive matrix formulation, topical film forming gel formulation, or topical film forming spray formulation.

The disclosure provides a method of treating, preventing, and/or slowing the onset or progression of a disease or condition and/or related symptom in a patient wherein the continuous delivery system comprises a pharmaceutical composition formulated as a transdermal patch, wherein the transdermal patch is selected from the group such as to reservoir patch, a multilayer transdermal matrix system, a microreservoir patch, a matrix patch, a drug in adhesive patch, a matrix patch of adhesive in combination with polymers, a pressure sensitive adhesive patch, extended-release transdermal film a liquid reservoir system, a microreservoir patch, a mucoadhesive patch, and combinations thereof. The disclosure provides a method of treating, preventing, and/or slowing the onset or progression of a disease or condition and/or related symptom in a patient wherein the continuous delivery system comprises a pharmaceutical composition formulated as a topical patch, wherein the topical patch is selected from the group such as a multilayer transdermal matrix system, reservoir patch, a microreservoir patch, a matrix patch, a drug in adhesive patch, a pressure sensitive adhesive patch, extended-release transdermal film a liquid reservoir system, a microreservoir patch, a mucoadhesive patch, a micro-dosing patch, a matrix patch of adhesive in combination with polymers, and combinations thereof. The disclosure provides a method of treating, preventing, and/or slowing the onset or progression of a disease or condition and/or related symptom in a patient wherein the continuous delivery system comprises a pharmaceutical composition formulated as a transdermal patch. The disclosure provides a method of treating, preventing, and/or slowing the onset or progression of a disease or condition and/or related symptom in a patient wherein the continuous delivery system comprises a pharmaceutical composition formulated as a multilayer transdermal matrix system, a metered dose transdermal gel, metered dose transdermal spray, a film forming gel, a film forming spray, or a meter-dose aerosol. The disclosure provides a method of treating, preventing, and/or slowing the onset or progression of a disease or condition and/or related symptom in a patient wherein the continuous delivery system comprises a pharmaceutical composition formulated as a topical patch. The disclosure provides a method of treating, preventing, and/or slowing the onset or progression of a disease or condition and/or related symptom in a patient wherein the continuous delivery system comprises a pharmaceutical composition formulated as metered dose gel, metered dose spray, gel, cream, solution, emulsion, liquid compositions, semisolid compositions, a matrix of adhesive in combination with polymers, or film forming formulation. The disclosure provides a method of treating, preventing, and/or slowing the onset or progression of a disease or condition and/or related symptom in a patient wherein the continuous delivery system comprises a pharmaceutical composition further comprising carriers or ingredients in effective amount selected from the group consisting of solvents, gelling agents, polymers, pressure sensitive adhesive polymers, penetration enhancers, emollients, skin irritation reducing agents, buffering agents, pH stabilizers, tackifier, diluent, bulking agent, solubilizers, suspending agents, dispersing agents, stabilizers, plasticizers, surfactants, antioxidants, oxidants, and combinations thereof.

The disclosure provides a method of treating, preventing, and/or slowing the onset or progression of a disease or condition and/or related symptom in a patient wherein the continuous delivery system comprises a pharmaceutical composition further comprising carriers or ingredients in effective amount selected from the group consisting of solvents, gelling agents, polymers, pressure sensitive adhesive polymers, penetration enhancers, emollients, skin irritation reducing agents, buffering agents, pH stabilizers, solubilizers, suspending agents, dispersing agents, stabilizers, plasticizers, tackifiers, diluents, bulking agents, surfactants, antioxidants, oxidants, and combinations thereof in the range of 0.1%-99.5% w/w or w/v. The disclosure provides a method of treating, preventing, and/or slowing the onset or progression of a disease or condition and/or related symptom in a patient wherein the continuous delivery system comprises a pharmaceutical composition formulated as a transdermal formulation which can be administered in a dosage regimen selected from the group consisting of once daily, twice daily, three times a day, once in 1-8 hrs, once in 1-24 hrs, once in two days, once in three days, once in four days, once in five days, once in six days, once in a week, once in a 8 to about 13 days, once in two weeks, and once in 15 days to about 30 days. The disclosure provides a method of treating, preventing, and/or slowing the onset or progression of a disease or condition and/or related symptom in a patient wherein the continuous delivery system comprises a pharmaceutical composition formulated as a topical formulation which can be administered in a dosage regimen selected from the group consisting of once daily, twice daily, three times a day, four times a day, five times a day, six times a day, once in 1-8 hrs, once in 1-24 hrs, once in two days, once in three days, once in four days, once in five days, once in six days, once in a week, once in a 8 to about 13 days, once in two weeks, and once in 15 days to about 30 days. The disclosure provides a method of treating, preventing, and/or slowing the onset or progression of a disease or condition and/or related symptom in a patient wherein the continuous delivery system provides a continuous, sustained delivery of the pharmaceutical composition to the patient via microneedles. The disclosure provides a method of treating, preventing, and/or slowing the onset or progression of a disease or condition and/or related symptom in a patient wherein the continuous delivery system provides a blood serum level of active agent selected from the group consisting of about 0.01 ng/mL, about 0.2 ng/mL, about 0.05 ng/mL, about 0.1 ng/mL, about 0.2 ng/mL, about 0.5 ng/mL, about 1 ng/mL, about 2 ng/mL, about 5 ng/mL, about 10 ng/mL, about 20 ng/mL, about 50 ng/mL, about 100 ng/mL, about 200 ng/mL, about 500 ng/mL, about 1 μg/mL, about 2 μg/mL, and about 5 μg/mL. The disclosure provides a method of treating, preventing, and/or slowing the onset or progression of a disease or condition and/or related symptom in a patient wherein the continuous delivery system comprises a pharmaceutical composition wherein active agent is present at a concentration selected from the group consisting of about 1% to about 10%, about 1% to about 9%, about 1% to about 8%, about 1% to about 7%, about 1% to about 6%, about 1% to about 5%, about 0.1 to about 50%, about 1 to 20%, of about 5% to 25%, about 10% to about 20%, or about 15% to about 18%, about 30% to about 70%, and about 35% to about 65% of the formulation.

The disclosure provides a method of treating, preventing, and/or slowing the onset or progression of a disease or condition and/or related symptom in a patient wherein the continuous delivery system comprises a pharmaceutical composition wherein active agent is present at a concentration selected from the group consisting of about 1% to about 10%, about 1% to about 9%, about 1% to about 8%, about 1% to about 7%, about 1% to about 6%, and about 1% to about 5% of the formulation. The disclosure provides a method of treating, preventing, and/or slowing the onset or progression of a disease or condition and/or related symptom in a patient wherein the continuous delivery system comprises a pharmaceutical composition wherein THC is selected from the group comprising of free base thereof, salts thereof, isomers thereof, amorphous forms thereof, crystalline forms thereof, co-crystalline forms thereof, prodrugs thereof, analogs thereof, derivatives thereof, synthetic forms thereof, biosynthetic forms thereof, naturally occurring forms thereof, active metabolites thereof, polymorph thereof, solid solution thereof, coated form thereof, stereoisomers thereof, solid solution thereof, ion-pair thereof, solution thereof, powder form thereof, liquid form thereof, alone or combinations thereof. The disclosure provides a method of treating, preventing, and/or slowing the onset or progression of a disease or condition and/or related symptom in a patient wherein the continuous delivery system comprises a pharmaceutical composition wherein CBD is selected from the group comprising of free base thereof, salts thereof, isomers thereof, amorphous forms thereof, crystalline forms thereof, co-crystalline forms thereof, prodrugs thereof, analogs thereof, derivatives thereof, synthetic forms thereof, biosynthetic forms thereof, naturally occurring forms thereof, active metabolites thereof, polymorph thereof, solid solution thereof, coated form thereof, ion-pairs thereof, stereoisomers thereof, solid solution thereof, solution thereof, powder form thereof, liquid form thereof, alone or combinations thereof. The disclosure provides a method of treating, preventing, and/or slowing the onset or progression of a disease or condition and/or related symptom in a patient wherein the continuous delivery system comprises a pharmaceutical composition which comprises one or more active agents selected from the group consisting of tetrahydrocannabinol (THC), cannabidiol (CBD), the free base thereof, salts thereof, isomers thereof, amorphous forms thereof, crystalline forms thereof, co-crystalline forms thereof, prodrugs thereof, analogs thereof, derivatives thereof, synthetic forms thereof, biosynthetic forms thereof, naturally occurring forms thereof, active metabolites thereof, polymorph thereof, solid solution thereof, coated form thereof, and combinations thereof, in a dosage form for transdermal delivery. The disclosure provides a method of treating, preventing, and/or slowing the onset or progression of a disease or condition and/or related symptom in a patient wherein the continuous delivery system comprises a pharmaceutical composition wherein said tetrahydrocannabinol (THC), cannabidiol (CBD), the free base thereof, salts thereof, isomers thereof, amorphous forms thereof, polymorphs forms thereof, stereoisomers thereof, ion-pairs thereof, coated forms thereof, crystalline forms thereof, co-crystalline forms thereof, prodrugs thereof, analogs thereof, derivatives thereof, synthetic forms thereof, biosynthetic forms thereof, naturally occurring forms thereof, active metabolites thereof, and combinations thereof is produced by a synthetic route. The disclosure provides a method of treating, preventing, and/or slowing the onset or progression of a disease or condition and/or related symptom in a patient wherein the continuous delivery system comprises a pharmaceutical composition wherein active agent is produced synthetically and has a purity equal to or greater than about 90%, 91%, 92%, 93%, 94%, 95%, 96%, 97%, 98%, 99%, 99.5%, 99.75%, or 100% (w/w) before being added to said pharmaceutical composition. The disclosure provides a method of treating, preventing, and/or slowing the onset or progression of a disease or condition and/or related symptom in a patient wherein the continuous delivery system comprises a pharmaceutical composition which is co-administered with at least one additional an active agent selected from the group consisting of: a dopamine precursor, a dopamine receptor agonist, and combinations thereof. The disclosure provides a method of treating, preventing, and/or slowing the onset or progression of a disease or condition and/or related symptom in a patient wherein the continuous delivery system comprises a pharmaceutical composition which is co-administered with at least one additional an active agent selected from the group consisting of: levodopa, bromocriptine, pergolide, pramipexole, cabergoline, ropinirole, apomorphine, and any combination thereof.

The disclosure provides a method of treating, preventing, and/or slowing the onset or progression of a disease or condition and/or related symptom in a patient wherein the disease or condition and/or related symptom is selected from the group consisting of cancer, pain, seizure disorder, Parkinson's Disease (“PD”), and a symptom of PD. The disclosure provides a method of treating, preventing, and/or slowing the onset or progression of a disease or condition and/or related symptom in a patient wherein the cancer is selected from the group consisting of endometrial cancer, breast cancer, ovarian cancer, cervical cancer, fallopian tube cancer, testicular cancer, primary peritoneal cancer, colon cancer, colorectal cancer, small intestine cancer, squamous cell carcinoma of the anogenital region, melanoma, renal cell carcinoma, lung cancer, non-small cell lung cancer, squamous cell carcinoma of the lung, stomach cancer, bladder cancer, gall bladder cancer, liver cancer, thyroid cancer, laryngeal cancer, salivary gland cancer, esophageal cancer, head and neck cancer, squamous cell carcinoma of the head and neck, prostate cancer, pancreatic cancer, mesothelioma, Merkel cell carcinoma, sarcoma, glioblastoma, GBM, and a hematological cancer, such as multiple myeloma, B-cell lymphoma, T-cell lymphoma, Hodgkin's lymphoma/primary mediastinal B-cell lymphoma, and chronic myelogenous leukemia. The disclosure provides a method of treating, preventing, and/or slowing the onset or progression of a disease or condition and/or related symptom in a patient wherein the cancer is glioblastoma, GBM. The disclosure provides a method of treating, preventing, and/or slowing the onset or progression of a disease or condition and/or related symptom in a patient wherein the pharmaceutical composition is co-administered with at least one additional therapeutic selected from the group consisting of: temozolomide, peptides, polypeptides, fusion proteins, nucleic acid molecules, small molecules, mimetic agents, synthetic drugs, inorganic molecules, organic molecules, chemotherapies, radiation therapies, hormonal therapies, anti-angiogenesis therapies, targeted therapies, and/or biological therapies including immunotherapies and surgery, and combinations thereof. The disclosure provides a method of treating, preventing, and/or slowing the onset or progression of a disease or condition and/or related symptom in a patient wherein the pain is chronic pain selected from the group consisting of neuropathic pain, peripheral neuropathic pain, inflammatory pain, musculoskeletal pain, pain due to muscle spasms, pain due to increased muscle tone, osteoarthritic pain, muscular headache, tension-type headache, migraine, cluster headache, atypical facial pain, referred pain, vulvodynia, proctodynia, and any combination thereof. The disclosure provides a method of treating, preventing, and/or slowing the onset or progression of a disease or condition and/or related symptom in a patient wherein the seizure disorder is selected from the group consisting of complex partial seizures, simple partial seizures, partial seizures with secondary generalization, generalized seizures (including absence, grand mal (tonic clonic), status epilepticus, tonic, atonic, myoclonic), neonatal and infantile spasms, drug-induced seizures, trauma-induced seizures, and febrile seizures, and additional specific epilepsy syndromes such as juvenile myoclonic epilepsy, Lennox-Gastaut, Dravet syndrome, Tuberous Sclerosis Complex (TSC), Treatment-Resistant Epilepsy, Treatment Resistant Pediatric Epilepsy, mesial temporal lobe epilepsy, nocturnal frontal lobe epilepsy, progressive epilepsy with mental retardation, and progressive myoclonic epilepsy, and seizures associated with CNS mass lesions. The disclosure provides a method of treating, preventing, and/or slowing the onset or progression of a disease or condition and/or related symptom in a patient wherein the seizure disorder is selected from the group consisting of complex partial seizures, simple partial seizures, partial seizures with secondary generalization, generalized seizures (including absence, grand mal (tonic clonic), status epilepticus, tonic, atonic, myoclonic), neonatal and infantile spasms, drug-induced seizures, trauma-induced seizures, and febrile seizures, and additional specific epilepsy syndromes such as juvenile myoclonic epilepsy, Lennox-Gastaut, Dravet syndrome, Tuberous Sclerosis Complex (TSC), Treatment-Resistant Epilepsy, Treatment Resistant Pediatric Epilepsy, mesial temporal lobe epilepsy, nocturnal frontal lobe epilepsy, progressive epilepsy with mental retardation, and progressive myoclonic epilepsy, as well as seizures associated with CNS mass lesions. The disclosure provides a method of treating, preventing, and/or slowing the onset or progression of a disease or condition and/or related symptom in a patient wherein the symptom of PD to be treated, prevented, and/or slowed is selected from the group consisting of: (a) at least one non-motor aspect of experiences of daily living as defined by Part I of the Unified Parkinson's Disease Rating Scale selected from the group consisting of cognitive impairment, hallucinations and psychosis, depressed mood, anxious mood, apathy, features of dopamine dysregulation syndrome, sleep problems, daytime sleepiness, pain, urinary problems, constipation problems, lightheadedness on standing, and fatigue; (b) at least one motor aspect of experiences of daily living as defined by Part II of the Unified Parkinson's Disease Rating Scale selected from the group consisting of speech, saliva and drooling, chewing and swallowing, eating tasks, dressing, hygiene, handwriting, turning in bed, tremors, getting out of a bed, a car, or a deep chair, walking and balance, and freezing; (c) at least one motor symptom identified in Part III of the Unified Parkinson's Disease Rating Scale selected from the group consisting of speech, facial expression, rigidity, finger tapping, hand movements, pronation-supination movements of hands, toe tapping, leg agility, arising from chair, gait, freezing of gait, postural stability, posture, body bradykinesia, postural tremor of the hands, kinetic tremor of the hands, rest tremor amplitude, and constancy of rest tremor; (d) at least one motor complication identified in Part IV of the Unified Parkinson's Disease Rating Scale selected from the group consisting of time spent with dyskinesias, functional impact of dyskinesias, time spent in the off state, functional impact of fluctuations, complexity of motor fluctuations, and painful off-state dystonia; (e) constipation; (f) depression; (g) cognitive impairment; (h) short or long term memory impairment; (i) concentration impairment; (j) coordination impairment; (k) mobility impairment; (l) speech impairment; (m) mental confusion; (n) sleep problem, sleep disorder, or sleep disturbance; (o) circadian rhythm dysfunction; (p) hallucinations; (q) fatigue; (r) REM disturbed sleep; (s) REM behavior disorder; (t) erectile dysfunction; (u) postural hypotension; (v) correction of blood pressure or orthostatic hypotension; (w) nocturnal hypertension; (x) regulation of temperature; (y) improvement in breathing or apnea; (z) correction of cardiac conduction defect; (aa) amelioration of pain; (bb) urinary incontinence, or restoration of bladder sensation and urination; (cc) mood swings; (dd) apathy; (ee) control of nocturia; (ff) neurodegeneration; (gg) anxiety; (hh) improving speaking ability, including public speaking ability; and/or (ii) Parkinson's Disease Dementia.

The disclosure provides a method of treating, preventing, and/or slowing the onset or progression of a disease or condition and/or related symptom in a patient wherein the PD symptom to be treated, prevented, and/or slowed is a sleep problem, sleep disorder, sleep disturbance, circadian rhythm dysfunction, REM disturbed sleep, or REM behavior disorder, and wherein: (a) the sleep disorder or sleep disturbance comprises a delay in sleep onset, sleep fragmentation, REM-behavior disorder, sleep-disordered breathing including snoring and apnea, day-time sleepiness, micro-sleep episodes, narcolepsy, hallucinations, or any combination thereof; and/or (b) the REM-behavior disorder comprises vivid dreams, nightmares, and acting out the dreams by speaking or screaming, or fidgeting or thrashing of arms or legs during sleep; and/or (c) treating the sleep problem, sleep disorder, sleep disturbance prevents or delays the onset and/or progression of the Parkinson's disease; and/or (d) the method results in a positive change in the sleeping pattern of the subject over a defined period of time; and/or (e) the method results in a positive change in the sleeping pattern of the subject over a defined period of time, wherein the positive change is defined as: (i) an increase in the total amount of sleep obtained of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (ii) a percent decrease in the number of awakenings during the night selected from the group consisting of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; and/or (f) as a result of the method the subject obtains the total number of hours of sleep recommended by a medical authority for the age group of the subject; and/or (g) wherein each defined period of time is independently selected from the group consisting of about 1 day to about 10 days, about 10 days to about 30 days, about 30 days to about 3 months, about 3 months to about 6 months, about 6 months to about 12 months, and about greater than 12 months.

The disclosure provides a method of treating, preventing, and/or slowing the onset or progression of a disease or condition and/or related symptom in a patient wherein the PD symptom to be treated, prevented, and/or slowed is hallucination and wherein: (a) the hallucination comprises a visual, auditory, tactile, gustatory or olfactory hallucination; and/or (b) treating the hallucination prevents and/or delays the onset and/or progression of the Parkinson's disease; and/or (c) the method results in a decreased number of hallucinations of the subject over a defined period of time; and/or (d) the method results in a decreased number of hallucinations of the subject over a defined period of time and the decrease in number is selected from the group consisting of by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (e) the method results in the subject being hallucination-free; and/or (f) the method results in a decreased severity of hallucinations of the subject over a defined period of time, as measured by one or more medically recognized technique; and/or (g) the method results in a decreased severity of hallucinations of the subject over a defined period of time and the decrease in severity is selected from the group consisting of by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%, as measured by one or more medically recognized technique; and/or (h) the medically recognized technique selected from the group consisting of Chicago Hallucination Assessment Tool (CHAT), The Psychotic Symptom Rating Scales (PSYRATS), Auditory Hallucinations Rating Scale (AHRS), Hamilton Program for Schizophrenia Voices Questionnaire (HPSVQ), Characteristics of Auditory Hallucinations Questionnaire (CAHQ), Mental Health Research Institute Unusual Perception Schedule (MUPS), positive and negative syndrome scale (PANS S), scale for the assessment of positive symptoms (SAPS), Launay-Slade hallucinations scale (LSHS), the Cardiff anomalous perceptions scale (CAPS), and structured interview for assessing perceptual anomalies (SIAPA); and/or (i) each defined period of time is independently selected from the group consisting of about 1 day to about 10 days, about 10 days to about 30 days, about 30 days to about 3 months, about 3 months to about 6 months, about 6 months to about 12 months, and about greater than 12 months.

The disclosure provides a method of treating, preventing, and/or slowing the onset or progression of a disease or condition and/or related symptom in a patient wherein the PD symptom to be treated, prevented, and/or slowed is depression and wherein: (a) treating the depression prevents and/or delays the onset and/or progression of the Parkinson's disease; and/or (b) the method results in improvement in a subject's depression over a defined period of time, as measured by one or more clinically-recognized depression rating scale; and/or (c) the method results in improvement in a subject's depression over a defined period of time, as measured by one or more clinically-recognized depression rating scale and the improvement is in one or more depression characteristics selected from the group consisting of mood, behavior, bodily functions such as eating, sleeping, energy, and sexual activity, and/or episodes of sadness or apathy; and/or (d) the method results in improvement in a subject's depression, as measured by one or more clinically-recognized depression rating scale, and the improvement a subject experiences following treatment is about 5, about 10, about 15, about 20, about 25, about 30, about 35, about 40, about 45, about 50, about 55, about 60, about 65, about 70, about 75, about 80, about 85, about 90, about 95 or about 100%; and/or (e) wherein each defined period of time is independently selected from the group consisting of about 1 day to about 10 days, about 10 days to about 30 days, about 30 days to about 3 months, about 3 months to about 6 months, about 6 months to about 12 months, and about greater than 12 months. The disclosure provides a method of treating, preventing, and/or slowing the onset or progression of a disease or condition and/or related symptom in a patient wherein the PD symptom to be treated, prevented, and/or slowed is cognitive impairment, and wherein: (a) treating the cognitive impairment prevents and/or delays the onset and/or progression of the Parkinson's disease; and/or (b) progression or onset of the cognitive impairment is slowed, halted, or reversed over a defined period of time following administration of the pharmaceutical composition, as measured by a medically-recognized technique; and/or (c) the cognitive impairment is positively impacted by the administration of the pharmaceutical composition, as measured by a medically-recognized technique; and/or (d) the cognitive impairment is positively impacted by the administration of the pharmaceutical composition, as measured by a medically-recognized technique and the positive impact on and/or progression of cognitive decline is measured quantitatively or qualitatively by one or more techniques selected from the group consisting of Mini-Mental State Exam (MMSE), Mini-cog test, and a computerized tested selected from Cantab Mobile, Cognigram, Cognivue, Cognision, or Automated Neuropsychological Assessment Metrics; and/or (e) the progression or onset of cognitive impairment is slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique; and/or (f) each defined period of time is independently selected from the group consisting of about 1 day to about 10 days, about 10 days to about 30 days, about 30 days to about 3 months, about 3 months to about 6 months, about 6 months to about 12 months, and about greater than 12 months.

The disclosure provides a method of treating, preventing, and/or slowing the onset or progression of a disease or condition and/or related symptom in a patient wherein the PD symptom to be treated, prevented, and/or slowed is constipation, and wherein: (a) treating the constipation prevents and/or delays the onset and/or progression of the Parkinson's disease; and/or (b) the administration of the pharmaceutical composition causes the subject to have a bowel movement; and/or (c) the method results in an increase in the frequency of bowel movement in the subject over a defined period of time; and/or (d) the method results in an increase in the frequency of bowel movement in the subject and the increase in the frequency of bowel movement is defined as: (i) an increase in the number of bowel movements per week of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (ii) a percent decrease in the amount of time between each successive bowel movement selected from the group consisting of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; and/or (e) as a result of the method the subject has the frequency of bowel movement recommended by a medical authority for the age group of the subject.

The disclosure provides a method of treating, preventing, and/or slowing the onset or progression of a disease or condition and/or related symptom in a patient wherein the PD symptom to be treated, prevented, and/or slowed is neurodegeneration correlated with PD, and wherein: (a) treating the neurodegeneration prevents and/or delays the onset and/or progression of the Parkinson's disease; and/or (b) the method results in treating, preventing, and/or delaying the progression and/or onset of neurodegeneration in the subject; and/or (c) progression or onset of the neurodegeneration is slowed, halted, or reversed over a defined period of time following administration of the pharmaceutical composition, as measured by a medically-recognized technique; and/or (d) the neurodegeneration is positively impacted by the administration of the pharmaceutical composition, as measured by a medically-recognized technique; and/or (e) the positive impact and/or progression of neurodegeneration is measured quantitatively or qualitatively by one or more techniques selected from the group consisting of electroencephalogram (EEG), neuroimaging, functional MM, structural MM, diffusion tensor imaging (DTI), [18F]fluorodeoxyglucose (FDG) PET, agents that label amyloid, [18F]F-dopa PET, radiotracer imaging, volumetric analysis of regional tissue loss, specific imaging markers of abnormal protein deposition, multimodal imaging, and biomarker analysis; and/or (f) the progression or onset of neurodegeneration is slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique; and/or (g) each defined period of time is independently selected from the group consisting of about 1 day to about 10 days, about 10 days to about 30 days, about 30 days to about 3 months, about 3 months to about 6 months, about 6 months to about 12 months, and about greater than 12 months.

The disclosure provides a continuous delivery system comprising a pharmaceutical composition comprising at least one active agent selected from the group consisting of: about 0.1% to about 50% of an active agent selected from the group consisting of cannabidiol (CBD), synthetic forms thereof, biosynthetic forms thereof, free base forms thereof, salts thereof, isomers thereof, amorphous forms thereof, derivatives thereof, and combinations thereof; about 0.1% to about 50% of an active agent selected from the group consisting of tetrahydrocannabinol (THC), synthetic forms thereof, biosynthetic forms thereof, free base forms thereof, salts thereof, isomers thereof, amorphous forms thereof, derivatives thereof, and combinations thereof; optionally, at least one additional active agent, and combinations thereof. The disclosure provides a continuous delivery system wherein the continuous delivery system provides a continuous, sustained delivery of the pharmaceutical composition to mitigate peak and valley pharmacokinetic behavior of the active agent. The disclosure provides a continuous delivery system wherein the continuous delivery system provides a continuous, sustained delivery of the pharmaceutical composition via administration to the patient by a route selected from the group consisting of parenteral, intravenous, subcutaneous, intramuscular, intrathecal, oral, buccal, mucosal, intranasal, rectal, vaginal, transdermal, implantable, topical, and combinations thereof. The disclosure provides a continuous delivery system wherein the continuous delivery system provides a continuous, sustained delivery of the pharmaceutical composition via intravenous or subcutaneous infusion. The disclosure provides a continuous delivery system wherein the continuous delivery system provides a continuous, sustained delivery of the pharmaceutical composition via subcutaneous infusion. The disclosure provides a continuous delivery system wherein the continuous delivery system provides a continuous, sustained delivery of the pharmaceutical composition via an infusion pump device.

The disclosure provides a continuous delivery system wherein the continuous delivery system provides a continuous, sustained delivery of the pharmaceutical composition via an ambulatory/portable infusion pump that is worn by the patient and may use replaceable cartridges. The disclosure provides a continuous delivery system wherein the continuous delivery system provides a continuous, sustained delivery of the pharmaceutical composition via a patch pump or micro pump. The disclosure provides a continuous delivery system wherein the continuous delivery system comprises a pharmaceutical composition selected from the group consisting of a liquid formulation, a solid formulation, a semi-solid formulation, an emulsion formulation, a nanoparticle formulation, a matrix formulation, a film formulation, a patch formulation, a formulation in an infusion pump, and/or combinations thereof. The disclosure provides a continuous delivery system wherein the continuous delivery system comprises a pharmaceutical composition selected from the group consisting of transdermal liquid formulation, transdermal semisolid formulation, transdermal gel formulation, or transdermal polymer matrix formulation, transdermal adhesive matrix formulation, transdermal film forming gel, transdermal film forming spray formulation, multilayer transdermal matrix system, transdermal drug-in-adhesive matrix formulation, and combinations thereof. The disclosure provides a continuous delivery system wherein the continuous delivery system comprises a pharmaceutical composition formulated as a topical liquid formulation, topical semisolid formulation, topical gel formulation, topical polymer matrix formulation, topical adhesive matrix formulation, topical film forming gel formulation, or topical film forming spray formulation. The disclosure provides a continuous delivery system wherein the continuous delivery system comprises a pharmaceutical composition formulated as a transdermal patch, wherein the transdermal patch is selected from the group such as to reservoir patch, a multilayer transdermal matrix system, a microreservoir patch, a matrix patch, a drug in adhesive patch, a matrix patch of adhesive in combination with polymers, a pressure sensitive adhesive patch, extended-release transdermal film a liquid reservoir system, a microreservoir patch, a mucoadhesive patch, and combinations thereof. The disclosure provides a continuous delivery system wherein the continuous delivery system comprises a pharmaceutical composition formulated as a topical patch, wherein the topical patch is selected from the group such as a multilayer transdermal matrix system, reservoir patch, a microreservoir patch, a matrix patch, a drug in adhesive patch, a pressure sensitive adhesive patch, extended-release transdermal film a liquid reservoir system, a microreservoir patch, a mucoadhesive patch, a micro-dosing patch, a matrix patch of adhesive in combination with polymers, and combinations thereof. The disclosure provides a continuous delivery system wherein the continuous delivery system comprises a pharmaceutical composition formulated as a transdermal patch. The disclosure provides a continuous delivery system wherein the continuous delivery system comprises a pharmaceutical composition formulated as a multilayer transdermal matrix system, a metered dose transdermal gel, metered dose transdermal spray, a film forming gel, a film forming spray, or a meter-dose aerosol. The disclosure provides a continuous delivery system wherein the continuous delivery system comprises a pharmaceutical composition formulated as a topical patch.

The disclosure provides a continuous delivery system wherein the continuous delivery system comprises a pharmaceutical composition formulated as metered dose gel, metered dose spray, gel, cream, solution, emulsion, liquid compositions, semisolid compositions, a matrix of adhesive in combination with polymers, or film forming formulation. The disclosure provides a continuous delivery system wherein the continuous delivery system comprises a pharmaceutical composition further comprising carriers or ingredients in effective amount selected from the group consisting of solvents, gelling agents, polymers, pressure sensitive adhesive polymers, penetration enhancers, emollients, skin irritation reducing agents, buffering agents, pH stabilizers, tackifier, diluent, bulking agent, solubilizers, suspending agents, dispersing agents, stabilizers, plasticizers, surfactants, antioxidants, oxidants, and combinations thereof. The disclosure provides a continuous delivery system wherein the continuous delivery system comprises a pharmaceutical composition further comprising carriers or ingredients in effective amount selected from the group consisting of solvents, gelling agents, polymers, pressure sensitive adhesive polymers, penetration enhancers, emollients, skin irritation reducing agents, buffering agents, pH stabilizers, solubilizers, suspending agents, dispersing agents, stabilizers, plasticizers, tackifiers, diluents, bulking agents, surfactants, antioxidants, oxidants, and combinations thereof in the range of 0.1%-99.5% w/w or w/v. The disclosure provides a continuous delivery system wherein the continuous delivery system comprises a pharmaceutical composition formulated as a transdermal formulation which can be administered in a dosage regimen selected from the group consisting of once daily, twice daily, three times a day, once in 1-8 hrs, once in 1-24 hrs, once in two days, once in three days, once in four days, once in five days, once in six days, once in a week, once in a 8 to about 13 days, once in two weeks, and once in 15 days to about 30 days. The disclosure provides a continuous delivery system wherein the continuous delivery system comprises a pharmaceutical composition formulated as a topical formulation which can be administered in a dosage regimen selected from the group consisting of once daily, twice daily, three times a day, four times a day, five times a day, six times a day, once in 1-8 hrs, once in 1-24 hrs, once in two days, once in three days, once in four days, once in five days, once in six days, once in a week, once in a 8 to about 13 days, once in two weeks, and once in 15 days to about 30 days. The disclosure provides a continuous delivery system wherein the continuous delivery system comprises a pharmaceutical composition which is formulated as microneedles. The disclosure provides a continuous delivery system wherein the continuous delivery system provides a blood serum level of active agent selected from the group consisting of about 0.01 ng/mL, about 0.02 ng/mL, about 0.05 ng/mL, about 0.1 ng/mL, about 0.2 ng/mL, about 0.5 ng/mL, about 1 ng/mL, about 2 ng/mL, about 5 ng/mL, about 10 ng/mL, about 20 ng/mL, about 50 ng/mL, about 100 ng/mL, about 200 ng/mL, about 500 ng/mL, about 1 μg/mL mL, about 2 μg/mL, and about 5 μg/mL. The disclosure provides a continuous delivery system wherein the continuous delivery system comprises a pharmaceutical composition wherein active agent is present at a concentration selected from the group consisting of about 1% to about 10%, about 1% to about 9%, about 1% to about 8%, about 1% to about 7%, about 1% to about 6%, about 1% to about 5%, about 0.1 to about 50%, about 1 to 20%, of about 5% to 25%, about 10% to about 20%, or about 15% to about 18%, about 30% to about 70%, and about 35% to about 65% of the formulation. The disclosure provides a continuous delivery system wherein the continuous delivery system comprises a pharmaceutical composition wherein active agent is present at a concentration selected from the group consisting of about 1% to about 10%, about 1% to about 9%, about 1% to about 8%, about 1% to about 7%, about 1% to about 6%, and about 1% to about 5% of the formulation.

The disclosure provides a continuous delivery system wherein the continuous delivery system comprises a pharmaceutical composition wherein THC is selected from the group comprising of free base thereof, salts thereof, isomers thereof, amorphous forms thereof, crystalline forms thereof, co-crystalline forms thereof, prodrugs thereof, analogs thereof, derivatives thereof, synthetic forms thereof, biosynthetic forms thereof, naturally occurring forms thereof, active metabolites thereof, polymorph thereof, solid solution thereof, coated form thereof, stereoisomers thereof, solid solution thereof, ion-pair thereof, solution thereof, powder form thereof, liquid form thereof, alone or combinations thereof. The disclosure provides a continuous delivery system wherein the continuous delivery system comprises a pharmaceutical composition wherein CBD is selected from the group comprising of free base thereof, salts thereof, isomers thereof, amorphous forms thereof, crystalline forms thereof, co-crystalline forms thereof, prodrugs thereof, analogs thereof, derivatives thereof, synthetic forms thereof, biosynthetic forms thereof, naturally occurring forms thereof, active metabolites thereof, polymorph thereof, solid solution thereof, coated form thereof, ion-pairs thereof, stereoisomers thereof, solid solution thereof, solution thereof, powder form thereof, liquid form thereof, alone or combinations thereof. The disclosure provides a continuous delivery system wherein the continuous delivery system comprises a pharmaceutical composition which comprises one or more active agents selected from the group consisting of tetrahydrocannabinol (THC), cannabidiol (CBD), the free base thereof, salts thereof, isomers thereof, amorphous forms thereof, crystalline forms thereof, co-crystalline forms thereof, prodrugs thereof, analogs thereof, derivatives thereof, synthetic forms thereof, biosynthetic forms thereof, naturally occurring forms thereof, active metabolites thereof, polymorph thereof, solid solution thereof, coated form thereof, and combinations thereof, in a dosage form for transdermal delivery. The disclosure provides a continuous delivery system wherein the continuous delivery system comprises a pharmaceutical composition wherein said tetrahydrocannabinol (THC), cannabidiol (CBD), the free base thereof, salts thereof, isomers thereof, amorphous forms thereof, polymorphs forms thereof, stereoisomers thereof, ion-pairs thereof, coated forms thereof, crystalline forms thereof, co-crystalline forms thereof, prodrugs thereof, analogs thereof, derivatives thereof, synthetic forms thereof, biosynthetic forms thereof, naturally occurring forms thereof, active metabolites thereof, and combinations thereof is produced by a synthetic route. The disclosure provides a continuous delivery system wherein the continuous delivery system comprises a pharmaceutical composition wherein active agent is produced synthetically and has a purity equal to or greater than about 90%, 91%, 92%, 93%, 94%, 95%, 96%, 97%, 98%, 99%, 99.5%, 99.75%, or 100% (w/w) before being added to said pharmaceutical composition. The disclosure provides a continuous delivery system wherein the continuous delivery system comprises a pharmaceutical composition which is co-administered with at least one additional an active agent selected from the group consisting of: a dopamine precursor, a dopamine receptor agonist, and combinations thereof. The disclosure provides a continuous delivery system wherein the continuous delivery system comprises a pharmaceutical composition which is co-administered with at least one additional an active agent selected from the group consisting of: levodopa, bromocriptine, pergolide, pramipexole, cabergoline, ropinirole, apomorphine, and any combination thereof.

The disclosure provides for the use of the compositions of the disclosure for the production of a medicament for preventing and/or treating the indications as set forth herein.

In accordance with a further embodiment, the present disclosure provides a use of the pharmaceutical compositions described above, in an amount effective for use in a medicament, and most preferably for use as a medicament for treating a disease or disorder, for example, as set forth in herein, in a subject.

In accordance with yet another embodiment, the present disclosure provides a use of the pharmaceutical compositions described above, and at least one additional therapeutic agent, in an amount effective for use in a medicament, and most preferably for use as a medicament for treating a disease or disorder associated with disease, for example, as set forth herein, in a subject.

The disclosure provides a method for treating and/or preventing a disease or condition as set forth herein in a patient, wherein said method comprises: selecting a patient in need of treating and/or preventing said disease or condition as set forth herein; administering to the patient a composition of the disclosure in a therapeutically effective amount, thereby treating and/or preventing said disease in said patient.

### DETAILED DESCRIPTION

As used herein the term “active pharmaceutical ingredient” (“API”) or “pharmaceutically active agent” is a drug or agent which can be employed as disclosed herein and is intended to be used in the human or animal body in order to heal, to alleviate, to prevent or to diagnose diseases, ailments, physical damage or pathological symptoms; allow the state, the condition or the functions of the body or mental states to be identified; to replace active substances produced by the human or animal body, or body fluids; to defend against, to eliminate or to render innocuous pathogens, parasites or exogenous substances or to influence the state, the condition or the functions of the body or mental states. Drugs in use can be found in reference works such as, for example, the Rote Liste or the Merck Index. Examples which may be mentioned include, for example, tretinoin.

As used herein, “pharmaceutically acceptable salts” refer to derivatives of the disclosed compounds wherein the therapeutic compound is modified by making acid or base salts thereof. Examples of pharmaceutically acceptable salts include, but are not limited to, mineral or organic acid salts of the active agent. The pharmaceutically acceptable salts include the conventional non-toxic salts, for example, from non-toxic inorganic or organic acids. For example, such conventional non-toxic salts include those derived from inorganic acids such as hydrochloric, hydrobromic, sulfuric, sulfonic, sulfamic, phosphoric, nitric and the like; and the salts prepared from organic acids such as amino acids, acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane di sulfonic, oxalic, isethionic, and other known to those of ordinary skill in the pharmaceutical sciences. Lists of suitable salts are found in texts such as Remington's Pharmaceutical Sciences, 18th Ed. (Alfonso R. Gennaro, ed.; Mack Publishing Company, Easton, Pa., 1990); Remington: the Science and Practice of Pharmacy 19^{th} Ed. (Lippincott, Williams & Wilkins, 1995); Handbook of Pharmaceutical Excipients, 3^{rd} Ed. (Arthur H. Kibbe, ed.; Amer. Pharmaceutical Assoc., 1999); the Pharmaceutical Codex: Principles and Practice of Pharmaceutics 12^{th} Ed. (Walter Lund ed.; Pharmaceutical Press, London, 1994); The United States Pharmacopeia: The National Formulary (United States Pharmacopeial Convention); and Goodman and Gilman's: the Pharmacological Basis of Therapeutics (Louis S. Goodman and Lee E. Limbird, eds.; McGraw Hill, 1992), the disclosures of which are hereby incorporated by reference.

An amount is “effective” as used herein, when the amount provides an effect in the subject. As used herein, the term “effective amount” means an amount of a compound or composition sufficient to significantly induce a positive benefit, including independently or in combinations the benefits disclosed herein, but low enough to avoid serious side effects, i.e., to provide a reasonable benefit to risk ratio, within the scope of sound judgment of the skilled artisan. For those skilled in the art, the effective amount, as well as dosage and frequency of administration, may be determined according to their knowledge and standard methodology of merely routine experimentation based on the present disclosure.

As used herein, the terms “subject” and “patient” are used interchangeably. As used herein, the term “patient” refers to an animal, preferably a mammal such as a non-primate (e.g., cows, pigs, horses, cats, dogs, rats etc.) and a primate (e.g., monkey and human), and most preferably a human. In some embodiments, the subject is a non-human animal such as a farm animal (e.g., a horse, pig, or cow) or a pet (e.g., a dog or cat). In a specific embodiment, the subject is an elderly human. In another embodiment, the subject is a human adult. In another embodiment, the subject is a human child. In yet another embodiment, the subject is a human infant.

As used herein, the phrase “pharmaceutically acceptable” means approved by a regulatory agency of the federal or a state government, or listed in the U.S. Pharmacopeia, European Pharmacopeia, or other generally recognized pharmacopeia for use in animals, and more particularly, in humans.

As used herein, the terms “prevent,” “preventing” and “prevention” in the context of the administration of a therapy to a subject refer to the prevention or inhibition of the recurrence, onset, and/or development of a disease or condition, or a combination of therapies (e.g., a combination of prophylactic or therapeutic agents).

As used herein, the terms “therapies” and “therapy” can refer to any method(s), composition(s), and/or agent(s) that can be used in the prevention, treatment and/or management of a disease or condition, or one or more symptoms thereof.

As used herein, the terms “treat,” “treatment,” and “treating” in the context of the administration of a therapy to a subject refer to the reduction or inhibition of the progression and/or duration of a disease or condition, the reduction or amelioration of the severity of a disease or condition, and/or the amelioration of one or more symptoms thereof resulting from the administration of one or more therapies.

As used herein, the term “continuous delivery,” “continuously administering,” or “continuous administration” refers to essentially uninterrupted administration to a subject of a medication or drug. The administration is non-stop (uninterrupted) except when it is necessary to refill the medication or drug supply or to administer the next dose in the regimen. “Continuous delivery” means there is an uninterrupted administration of the medication or drug, and that the dosing rate or absorbance rate may fluctuate over the dosing interval.

As used herein, the term “multi-particulates” refers to one or more unit dosage systems such as, but not limited to, pellets, beads, spheres, mini-tablets, seeds, spheroids or granules with modified drug release profile. The multi-particulates comprise a drug-release controlling and/or drug-protecting film or matrix, such as a polymeric film or matrix, whose intactness or efficiency is susceptible to certain conditions such as heat or mechanical forces that may occur during post-processing. The expression “core material” describes the nature of the interior part of multi-particulates that may also comprise a functional coat. Exemplary “core-materials” may be pellets (spherical matrix systems that contain a drug and further excipients), granules (less spherical particles that are almost entirely composed of drug) or nonpareils (spherical particles without drug).

As used herein, the term “about” when used in conjunction with a stated numerical value or range has the meaning reasonably ascribed to it by a person skilled in the art, i.e., denoting somewhat more or somewhat less than the stated value or range.

### Active Agent

The term “active agent” or “active ingredient” refers to an agent, active ingredient compound or other substance, or compositions and mixture thereof that provide some pharmacological, often beneficial, effect. Reference to a specific active ingredient shall include where appropriate the active ingredient and its pharmaceutically acceptable salts. The disclosure provides for, for example, continuous drug delivery systems and methods comprising one or more of the following active agents, for example, tetrahydrocannabinol (THC), cannabidiol (CBD), and combinations thereof.

The term “active agent” or “active ingredient” refers to an agent, active ingredient compound or other substance, or compositions and mixture thereof that provide some pharmacological, often beneficial, effect. Reference to a specific active ingredient shall include where appropriate the active ingredient and its pharmaceutically acceptable salts. The disclosure provides for, for example, continuous drug delivery systems and methods comprising one or more of the following active agents: Cannabinoids are a group of 21-carbon-containing terpenophenolic compounds produced by Cannabis species. Cannabinoids may also be synthetically produced. The term “cannabinoid” refers hereinafter to a class of diverse chemical compounds that act on cannabinoid receptors on cells that repress neurotransmitter release in the brain. These receptor proteins include the endocannabinoids (produced naturally in the body by humans and animals), the phytocannabinoids (found in cannabis and some other plants), and synthetic cannabinoids. Lipophilic cannabinoids are generally grouped as endocannabinoids (most typically as mammalian endocannabinoids); phytocannabinoids, from plant sources; and synthetic cannabinoids. Such cannabinoids are also often classified into the following subclasses: Cannabigerols (CBG); Cannabichromenes (CBC); Cannabidiol (CBD; CBDL); Tetrahydrocannabinol (THC); Cannabinol (CBN); Cannabicyclol (CBL); Cannabielsoin (CBE); and, Cannabitriol (CBT).

Cannabidiol IUPAC Name 2-[(1R,6R)-6-isopropenyl-3-methylcyclohex-2-en-1-yl]-5-pentylbenzene-1,3-diol Chemical Formula: C₂₁H₃₀O₂ Molecular weight: 314.46 dalton

Chemical structure is shown below as formula I

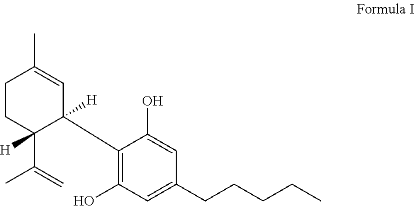 [SMILES: C=C(C)[C@@H]1CCC(C)=C[C@H]1c1c(O)cc(CCCCC)cc1O]

Tetrahydrocannbinol (THC) IUPAC Name (−)-(6aR,10aR)-6,6,9-Trimethyl-3-pentyl-6a,7,8,10a-tetrahydro-6H-benzo[c]chromen-1-ol Chemical Formula: C₂₁H₃₀O₂ Molecular weight: 314.47 dalton.

Chemical structure is shown below as formula II

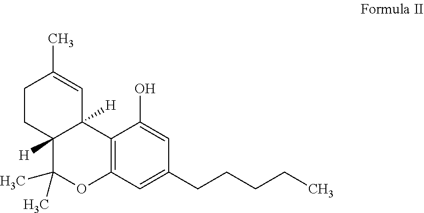 [SMILES: CCCCCc1cc(O)c2c(c1)OC(C)(C)[C@@H]1CCC(C)=C[C@@H]21]

As used herein, the word cannabis refers to all pharmaceutically acceptable forms of cannabis and its derivatives either alone or in combinations thereof, for example, in following forms but not limited to such as free base or salts or isomers or amorphous or crystalline or co crystalline or solid solution or prodrugs or analogs or derivatives or metabolites or polymorphs or its stereoisomer or coated form or ion-pairs. For example, cannabidiol's free base or its salts or its isomers or its amorphous form or its crystalline form or its co crystalline form or its solid solution or its prodrugs or its analogs or its derivatives or synthetic forms or its polymorphs or its stereoisomer or its ion-pairs. The compound may be in the form of, for example, a pharmaceutically acceptable salt, such as an acid addition salt or a base salt, or a solvate thereof, including a hydrate thereof. Suitable acid addition salts are formed from acids which form non-toxic salts and examples are the hydrochloride, hydrobromide, hydroiodide, sulphate, bisulphate, nitrate, phosphate, hydrogen phosphate, acetate, maleate, fumarate, lactate, tartrate, citrate, gluconate, succinate, saccharate, benzoate, methanesulphonate, ethanesulphonate, benzenesulphonate, p-toluenesulphonate and pamoate salts. Suitable base salts are formed from bases which form non-toxic salts and examples are the sodium, potassium, aluminium, calcium, magnesium, zinc and diethanolamine salts.

As used herein, the term “cannabidiol” includes the free base thereof, salts thereof, isomers thereof, amorphous forms thereof, crystalline forms thereof, co crystalline forms thereof, prodrugs thereof, analogs thereof, derivatives thereof, synthetic forms thereof, biosynthetic forms thereof, active metabolites thereof, solid solution thereof, polymorph thereof, stereoisomers thereof, powder form thereof, liquid form thereof, ion-pairs thereof, solution of cannabidiol in solvents such as but not limited to methanol, etc. alone or in combinations thereof.

As used herein, the term “THC” includes the free base thereof, salts thereof, isomers thereof, amorphous forms thereof, crystalline forms thereof, co crystalline forms thereof, prodrugs thereof, analogs thereof, derivatives thereof, synthetic forms thereof, biosynthetic forms thereof, active metabolites thereof, solid solution thereof, powder form thereof, liquid form thereof, ion-pairs thereof, polymorph thereof, stereoisomers thereof, solution of THC in solvents such as but not limited to methanol, heptane, etc. alone or in combinations thereof.

As used herein, synthetic cannabinoids include at least the following: AM-087 is an analgesic drug that is a cannabinoid agonist derivative of Δ8THC substituted on the 3-position side chain and a potent CB1 agonist; AM-251 is an inverse agonist at the CB1 cannabinoid receptor with close structural similarity to SR141716A (rimonabant), both of which are biarylpyrazole cannabinoid receptor antagonists as well as μ-opioid receptor antagonist; Methanandamide (AM-356) is a stable chiral analog of anandamide and acts on the cannabinoid receptors with a Ki of 17.9 nM at CB1 and 868 nM at CB2; AM-374—palmitylsulfonyl fluoride; AM-381—stearylsulfonyl fluoride; AM404, also known as N-arachidonoylaminophenol, is an active metabolite of paracetamol (acetaminophen) thought to induce its analgesic action through its activity on the endocannabinoid, COX, and TRPV systems, all of which are present in pain and thermoregulatory pathways; AM-411 is an analgesic that is a cannabinoid agonist; AM-411 is a potent and fairly selective CB1 full agonist and produces similar effects to other cannabinoid agonists such as analgesia, sedation, and anxiolysis; AM-630 (6-lodopravadoline) acts as a potent and selective inverse agonist for the cannabinoid receptor CB2, selectivity over CB1 where it acts as a weak partial agonist; AM-661—1-(N-methyl-2-piperidine)methyl-2-methyl-3-(2-iodo)benzoylindole; MH-018 (1-pentyl-3-(1-naphthoyl)indole) or AM-678 is an analgesic chemical from the naphthoylindole family that acts as a full agonist at both the CB 1 and CB2 cannabinoid receptors, with some selectivity for CB2; AM-679 acts as a moderately potent agonist for the cannabinoid receptors; AM-694 (1-(5-fluoropentyl)-3-(2-iodobenzoyl)indole) acts as a potent and selective agonist for the cannabinoid receptor CB1; AM-735—3-bornyl-Δ8-THC, a mixed CB1/CB2 agonist; AM-855 is an analgesic cannabinoid agonist at both CB1 and CB2 with moderate selectivity for CB1; AM-881—a chlorine-substituted stereoisomer of anandamide whose Ki=5.3 nM at CB 1 and 95 nM at CB2; AM-883 an allyl-substituted stereoisomer of anandamide whose Ki=9.9 nM at CB1 and 226 nM at CB2; AM-905 is an analgesic cannabinoid which acts as a potent and reasonably selective agonist for the CB 1 cannabinoid receptor; AM-906 is an analgesic drug which is a cannabinoid agonist and is a potent and selective agonist for the CB1 cannabinoid receptor; AM-919 is an analgesic cannabinoid receptor agonist, potent with respect to both CB1 and CB2; AM-926—a potent agonist at both CB1 and CB2 with moderate selectivity for CB1; AM-938 is an analgesic drug which is a cannabinoid receptor agonist and while it is still a potent agonist at both CB1 and CB2, it is reasonably selective for CB2; AM-1116—a dimethylated stereoisomer of anandamide; AM-1172—an endocannabinoid analog specifically designed to be a potent and selective inhibitor of AEA uptake that is resistant to FAAH hydrolysis; AM-1220 is a potent and moderately selective agonist for the cannabinoid receptor CB1; AM-1221 acts as a potent and selective agonist for the cannabinoid receptor CB2; AM-1235 (1-(5-fluoropentyl)-3-(naphthalen-1-oyl)-6-nitroindole) acts as a potent and reasonably selective agonist for the cannabinoid receptor CB 1; AM-1241 (1-(methylpiperidin-2-ylmethyl)-3-(2-iodo-5-nitrobenzoyl)indole) is a potent and selective agonist for the cannabinoid receptor CB2, with analgesic effects in mammals, particularly against “atypical” pain such as hyperalgesia and allodynia, and has also shown efficacy in the treatment of amyotrophic lateral sclerosis in mammalian models; AM-1248 acts as a moderately potent agonist for both the cannabinoid receptors CB1 and CB2; AM-1710—a CB2 selective cannabilactone with 54× selectivity over CB1; AM-1714 acts as a reasonably selective agonist of the peripheral cannabinoid receptor CB2 and has both analgesic and anti-allodynia effects; AM-2201 (1-(5-fluoropentyl)-3-(1-naphthoyl)indole) acts as a potent but nonselective full agonist for the cannabinoid receptor; AM-2212—a potent agonist at both CB1 and CB2; AM-2213—a potent agonist at both CB1 and CB2; AM-2232 (1-(4-cyanobutyl)-3-(naphthalen-l-oyl)indole) acts as a potent but unselective agonist for the cannabinoid receptors CB1 and CB2; AM-2233 acts as a highly potent full agonist for the cannabinoid receptors CB1 and CB2 and has been found to fully substitute for THC in certain mammalian studies, with a potency lower than that of JWH-018 but higher than WIN 55, 212-2; AM-2389 acts as a potent and reasonably selective agonist for the CB1 receptor; AM-3102—an analog of oleoylethanolamide, (the endogenous agonist for proliferator-activated receptor a (PPARα)) it acts as a weak cannabinoid agonist at CB1 and at CB2; AM-4030 an analgesic which is potent agonist at both CB1 and CB2, but also reasonably selective for CB1; AM-4054 is a potent but slow-onset agonist with CB1 affinity and selectivity CB1 over CB2; AM-4113—a CB1 selective neutral antagonist; AM-6545 acts as a peripherally selective silent antagonist for the CB1 and was developed for the treatment of obesity; JWH-007—an analgesic which acts as a cannabinoid agonist at both the CB1 receptor and CB2 receptors, with some selectivity for CB2, JWH-007 is an analgesic which acts as a cannabinoid agonist at both the CB1 and CB2 receptors; JWH-015 acts as a subtype-selective cannabinoid agonist which binds almost 28× more strongly to CB2 than CB1. and has been shown to have immunomodulatory effects, and may be useful in the treatment and/or prevention and/or control of cancer, such as glioblastoma, GBM, JWH-018 an analgesic which acts as a full agonist at both the CB1 and CB2 cannabinoid receptors and produces effects similar to those of THC; JWH-019—an agonist at both CB1 and CB2 receptors and is an analgesic from the naphthoylindole family that acts as a cannabinoid agonist at both the CB1 and CB2 receptors; JWH-030—an analgesic which is a partial agonist at CB1 receptors; JWH-047—a potent and selective agonist for the CB2 receptor, JWH-048—a potent and selective agonist for the CB2 receptor, JWH-051—an analgesic with a high affinity for the CB1 receptor, but is a much stronger agonist for CB2, JWH-057—a 1-deoxy analog of Δ8-THC that has very high affinity for the CB2 receptor, but also has high affinity for the CB1 receptor; JWH-073—an analgesic which acts as a cannabinoid agonist at both the CB1 and CB2 receptors. It is somewhat selective for the CB1 subtype; JWH-081—an analgesic which acts as an agonist at both the cannabinoid CB1 AND CB2 receptors; JWH-098—a potent and fairly selective CB2 agonist; JWH-116—a CB1 ligand; JWH-120—a potent and 173-fold selective CB2 agonist; JWH-122—a potent and fairly selective CB1 agonist; JWH-133—a potent and highly selective CB2 receptor agonist; 1JWH-139—3-(1,1-dimethylpropyl)-6,6,9-trimethyl-6a,7,10,10a-tetrahydro-6H-benzo[c]chromene; JWH-147—an analgesic from the naphthoylpyrrole family, which acts as a cannabinoid agonist at both the CB1 and CB2 receptors; JWH-148—a moderately selective ligand for the CB2 receptor, with more than 8 times selectivity over the CB1 subtype; JWH-149—a potent and fairly selective CB2 agonist; JWH-161—a CB1 ligand; JWH-164—a potent cannabinoid agonist; JWH-166—a potent and highly selective CB2 agonist; JWH-167—a weak cannabinoid agonist from the phenylacetylindole family; JWH-171—an analgesic which acts as a cannabinoid receptor agonist; JWH-175—(1-pentylindol-3-yl)naphthalen-1-ylmethane, 22 nM at CB1, JWH-176—1-([(1E)-3-pentylinden-1-ylidine]methyl)naphthalene; JWH-181—a potent cannabinoid agonist; JWH-182—a potent cannabinoid agonist with some selectivity for CB1; JWH-184—1-pentyl-1H-indol-3-yl-(4-methyl-1-naphthyl)methane; JWH-185—1-pentyl-1H-indol-3-yl-(4-methoxy-1-naphthyl)methane; JWH-192—(1-(2-morpholin-4-yl ethyl)indol-3-yl)-4-methylnaphthalen-1-ylmethane; JWH-193—(1-(2-morpholin-4-ylethyl)indol-3-yl)-4-methylnaphthalen-1-ylmethanone; JWH-194—2-methyl-1-pentyl-1H-indol-3-yl-(4-methyl-1-naphthyl)methane; JWH-195—(1-(2-morpholin-4-ylethyl)indol-3-yl)-naphthalen-1-ylmethane; JWH-196—2-methyl-3-(1-naphthalenylmethyl)-1-pentyl-1H-Indole; JWH-197—2-methyl-1-pentyl-1H-indol-3-yl-(4-methoxy-1-naphthyl)methane; JWH-198—(1-(2-morpholin-4-ylethyl)indol-3-yl)-4-methoxynaphthalen-1-ylmethanone; JWH-199—(1-(2-morpholin-4-ylethyl)indol-3-yl)-4-methoxynaphthalen-1-ylmethane; JWH-200—an analgesic from the aminoalkylindole family, which acts as a cannabinoid receptor agonist; JWH-203—an analgesic from the phenylacetylindole family, which acts as a cannabinoid agonist with approximately equal affinity at both the CB1 and CB2 receptors; JWH-205—142-methyl-1-pentylindol-3-yl)-2-phenylethanone; JWH-210—an analgesic chemical from the naphthoylindole family, which acts as a potent cannabinoid agonist at both the CB1 and CB2 receptors; JWH-213—a potent and fairly selective CB2 agonist; JWH-229—1-methoxy-3-(1′,1′-dimethylhexyl)-Δ8-THC, a dibenzopyran cannabinoid which is a potent CB2 agonist; JWH-234—a cannabinoid agonist with selectivity for CB2; JWH-250—an analgesic from the phenylacetylindole family, which acts as a cannabinoid agonist at both the CB1 and CB2 receptors; JWH-251—(1-pentyl-3-(2-methylphenylacetyl)indole); JWH-258—a potent and mildly selective CB1 agonist; JWH-302-(1-pentyl-3-(3-methoxyphenylacetyl)indole); JWH-307—an analgesic from the naphthoylpyrrole family, which acts as a cannabinoid agonist at both the CB1 and CB2 receptors that is somewhat selective for the CB2 subtype; JWH-350—a 11-nor-1-methoxy-3-(1′,1′-dimethylheptyl)-9α-hydroxyhexahydrocannabinol has a 33-fold selectivity for the CB2 receptor and high CB2receptor affinity with little affinity for the CB1 receptor; JWH-359—a dibenzopyran cannabinoid that is a potent and selective CB2 receptor agonist; JWH-387—1-pentyl-3-(4-bromo-1-naphthoyl)indole, an analgesic from the naphthoylindole family, which acts as a potent cannabinoid agonist at both receptors CB1 and CB2; JWH-398—an analgesic chemical from the naphthoylindole family, which acts as a potent cannabinoid agonist at both receptors with a Ki of 2.3 nM at CB1 and 2.8 nM at CB2; JWH-424—a potent and moderately selective CB2 agonist with a Ki of 5.44 nM at CB2 and 20.9 nM at CB1; HU-210 is a cannabinoid that is 100 to 800 times more potent than natural THC from cannabis and has an extended duration of action and is a potent analgesic with many of the same effects as natural THC; Ajulemic acid (AB-III-56, HU-239, IP-751, CPL 7075, CT-3, Resunab) is a cannabinoid derivative of the non-psychoactive THC metabolite 11-nor-9-carboxy-THC that shows useful analgesic and anti-inflammatory effects without causing a subjective “high”. It is being developed for the treatment of neuropathic pain and inflammatory conditions such as arthritis and for the treatment of orphan life-threatening inflammatory diseases; HU-243 (AM-4056) is a cannabinoid which is a potent agonist at both the CB1 and CB2 receptors; HU-308 acts as a cannabinoid agonist and is highly selective for the CB2 receptor subtype. It has analgesic effects, promotes proliferation of neural stem cells, and protects both liver and blood vessel tissues against oxidative stress via inhibition of TNF-α; HU-331 is a quinone anticarcinogenic synthesized from cannabidiol; HU-336 is a strongly antiangiogenic compound, it inhibits angiogenesis by directly inducing apoptosis of vascular endothelial cells without changing the expression of pro- and anti-angiogenic cytokines and their receptors; HU-345 (cannabinol quinone) is a drug that is able to inhibit aortic ring angiogenesis more potently than its parent compound cannabinol; CP 47,497 or (C7)-CP 47,497 is a cannabinoid receptor agonist drug.

The disclosure also provides methods for the biosynthesis of cannabinoids and for the use of a eukaryotic or prokaryotic expression system for the production of biosynthetic enzymes that can be used for the manufacture of cannabinoids and cannabinoid analogs. Yeast as well as eukaryotic and prokaryotic cells are suitable for the cloning and expression of the cannabinoid acid synthase enzymes and include without limitation E coli, yeast and baculovirus hosts. Thus, the present disclosure provides a method for the production of biosynthetic cannabinoids, such as for example THC and/or CBD, using cannabinoid acid synthase enzymes including, but not limited to, tetrahydrocannabinolic acid (THCA) synthase and cannabidiolic acid (CBDA) synthase. The disclosure further provides for continuous drug delivery systems and methods as disclosed herein comprising, for example, biosynthetic CBD, alone or in combination with other active agents.

According to certain embodiments, continuous drug delivery systems and methods described herein are for the prevention and/or control and/or treatment of cancer.

According to certain embodiments described herein, pharmaceutical composition or continuous drug delivery systems and methods contains cannabidiol and/or THC—the free base thereof, salts thereof, isomers thereof, amorphous forms thereof, crystalline forms thereof, co crystalline forms thereof, prodrugs thereof, analogs thereof, derivatives thereof, synthetic forms thereof, biosynthetic forms thereof, active metabolites thereof, polymorphs thereof, stereoisomers thereof, coated form thereof, solid solution thereof, ion-pairs thereof, solution thereof in solvents alone or in combinations thereof. More preferably continuous drug delivery systems and methods may include cannabidiol, the free base thereof, salts thereof, isomers thereof, amorphous forms thereof, crystalline forms thereof, co crystalline forms thereof, prodrugs thereof, analogs thereof, derivatives thereof, synthetic forms thereof, biosynthetic forms thereof, active metabolites thereof, polymorphs thereof, ion-pairs thereof, stereoisomers thereof, coated form thereof, solution of cannabidiol in methanol, alone or in combinations thereof. More preferably continuous drug delivery systems and methods may include THC, the free base thereof, salts thereof, isomers thereof, amorphous forms thereof, crystalline forms thereof, co crystalline forms thereof, prodrugs thereof, analogs thereof, derivatives thereof, ion-pairs thereof, synthetic forms thereof, biosynthetic forms thereof, active metabolites thereof, polymorphs thereof, stereoisomers thereof, coated form thereof, solution of cannabidiol in methanol, alone or in combinations thereof.

As used herein, the word active agent refers to all pharmaceutically acceptable forms of the active agent and its derivatives either alone or in combinations thereof, for example, in following forms but not limited to such as free base or salts or isomers or amorphous or crystalline or co crystalline or solid solution or prodrugs or analogs or derivatives or metabolites. For example, the active agent's free base or its salts or its isomers or its amorphous form or its crystalline form or its co crystalline form or its solid solution or its prodrugs or its analogs or its derivatives or synthetic forms. The compound may be in the form of, for example, a pharmaceutically acceptable salt, such as an acid addition salt or a base salt, or a solvate thereof, including a hydrate thereof. Suitable acid addition salts are formed from acids which form non-toxic salts and examples are the hydrochloride, hydrobromide, hydroiodide, sulphate, bisulphate, nitrate, phosphate, hydrogen phosphate, acetate, maleate, fumarate, lactate, tartrate, citrate, gluconate, succinate, saccharate, benzoate, methanesulphonate, ethanesulphonate, benzenesulphonate, p-toluenesulphonate and pamoate salts. Suitable base salts are formed from bases which form non-toxic salts and examples are the sodium, potassium, aluminium, calcium, magnesium, zinc and diethanolamine salts. The active ingredient(s) can be present in the form of a free base or in the form of pharmaceutically acceptable salts. Pharmaceutically acceptable salts forming part of this invention are intended to define but not limited to salts of the carboxylic acid moiety such as alkali metal salts like Li, Na and K salts; alkaline earth metal salts like Ca and Mg salts; salts of organic bases such as lysine, arginine, guanidine, diethanolamine, choline, and the like; ammonium or substituted ammonium salts and aluminium salts. Salts may be acid addition salts which defines but not limited to sulfates, nitrates, phosphates, perchlorates, borates, hydrohalides, acetates, tartrates, maleates, citrates, succinates, palmoates, methanesulfonates, benzoates, salicylates, hydroxynaphthoates, benzensulfonates, ascorbates, glycerophosphates, ketoglutarates and the like.

As used herein, the term “active agent” includes the free base thereof, salts thereof, isomers thereof, amorphous forms thereof, crystalline forms thereof, co crystalline forms thereof, prodrugs thereof, analogs thereof, derivatives thereof, synthetic forms thereof, alone or in combinations thereof. In certain embodiments the active agent is highly purified. In certain embodiments the active agent is present as a highly purified extract of active agent which comprises at least 90%, 91%, 92%, 93%, 94%, 95%, 96%, 97%, 98%, 99%, 99.5%, or 99.75% (w/w) of the formulation in certain embodiments, the dose of active agent is greater than, for example, about 1, 2, 3, 4, 5, 6, 7, 8, 9, 10, 15, 20, 25, 30, 35, 40, or 45 mg/kg/day. In certain embodiments, the dose of active agent is greater than, for example, about 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, or 275 mg/day. In exemplary embodiments, formulations of the disclosure may comprise active agent at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80% of the formulation. In exemplary embodiments, formulations of the disclosure may comprise active agent at a concentration of about 1 to 20%, of about 5% to 25%, about 10% to about 20%, or about 15% to about 18%, about 30% to about 70%, about 35% to about 65%, about 63.13%, and about 40% to about 64% w/w. In exemplary formulations of the disclosure, the active agent will represent approximately 1 wt % to 75 wt %, preferably 2 wt % to 30 wt %, more preferably 5 wt. % to 20 wt. % of the formulation.

In exemplary embodiments, formulations as disclosed herein may comprise active agent at a concentration of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80%, In exemplary embodiments, formulations as disclosed herein may comprise active agent at a concentration of about 1 to about 20%, of about 5% to about 25%, about 10% to about 20%, or about 15% to about 18%, about 30% to about 70%, about 35% to about 65%, about 63.13%, and about 40% to about 64% w/w.

In certain embodiments the active agent is present as a highly purified extract of active agent which comprises at least 90%, 91%, 92%, 93%, 94%, 95%, 96%, 97%, 98%, 99%, 99.5%, or 99.75% (w/w) of the formulation.

In certain embodiments the active agent is present in the formulation provided at a concentration of at least 90%, 91%, 92%, 93%, 94%, 95%, 96%, 97%, 98%, 99%, 99.5%, or 99.75%, or 100% (w/w).

In certain embodiments the active agent is 100% synthetic. In certain embodiments the active agent has a purity equal to or greater than about 90%, 91%, 92%, 93%, 94%, 95%, 96%, 97%, 98%, 99%, 99.5%, 99.75%, or 100% (w/w). In certain embodiments the active agent is produced synthetically and has a purity equal to or greater than about 90%, 91%, 92%, 93%, 94%, 95%, 96%, 97%, 98%, 99%, 99.5%, 99.75%, or 100% (w/w). In certain embodiments the active agent is a combination of active agents, and each active agent may be produced synthetically and independently have a purity equal to or greater than about 90%, 91%, 92%, 93%, 94%, 95%, 96%, 97%, 98%, 99%, 99.5%, 99.75%, or 100% (w/w).

In certain embodiments, the dose of active agent is equal to or greater than, for example, about 0.001, 0.0025 0.005, 0.0075, 0.01, 0.025, 0.05, 0.075, 0.1, 0.25, 0.75, 1, 2, 3, 4, 5, 6, 7, 8, 9, 10, 15, 20, 25, 30, 35, 40, or 45 mg/kg/day. In certain embodiments, the dose of active agent is equal to or greater than, for example, about 0.001 ng/day, 0.01 ng/day, 0.025 ng/day. 0.05 ng/day, 0.1 ng/day, 0.25 ng/day, 0.5 ng/day, 1 ng/day, 10 ng/day, 25 ng/day, 50 ng/day, 100 ng/day, 250 ng/day, 500 ng/day, 1000 ng/day, 0.001 microgram/day, 0.01 microgram/day, 0.025 microgram/day, 0.050 microgram/day, 0.1 microgram/day, 0.25 microgram/day, 0.5 microgram/day, 1 microgram/day, 2.5 microgram/day, 5 microgram/day, 10 microgram/day, 25 microgram/day, 50 microgram/day, 100 microgram/day, 250 microgram/day, or 500 microgram/day. In certain embodiments, the dose of active agent is equal to or greater than, for example, about 0.001, 0.0025 0.005, 0.0075, 0.01, 0.025, 0.05, 0.075, 0.1, 0.25, 0.75, 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, or 275 ng/day. In certain embodiments, the dose of active agent is equal to or greater than, for example, about 0.001, 0.0025 0.005, 0.0075, 0.01, 0.025, 0.05, 0.075, 0.1, 0.25, 0.75, 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, or 275 mg/day. In exemplary embodiments, formulations of the disclosure may comprise active agent at a concentration of about 0.001 ng, 0.01 ng, 0.025 ng. 0.05 ng, 0.1 ng, 0.25 ng, 0.5 ng, 1 ng, 10 ng, 25 ng, 50 ng, 100 ng, 250 ng, 500 ng, 1000 ng, 0.001 microgram, 0.01 microgram, 0.025 microgram. 0.05 microgram, 0.1 microgram, 0.25 microgram, 0.5 microgram, 1 microgram, 2.5 microgram, 5 microgram, 10 microgram, 25 microgram, 50 microgram, 100 microgram, 250 microgram, or 500 microgram. In exemplary embodiments, formulations of the disclosure may comprise active agent at a concentration of about 0.001, 0.0025 0.005, 0.0075, 0.01, 0.025, 0.05, 0.075, 0.1, 0.25, 0.75, 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, or 275 ng. In exemplary embodiments, formulations of the disclosure may comprise active agent at a concentration of about 0.001, 0.0025 0.005, 0.0075, 0.01, 0.025, 0.05, 0.075, 0.1, 0.25, 0.75, 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, or 275 mg.

In an exemplary formulation as disclosed herein, the active agent will represent approximately 1 wt % to 75 wt %, preferably 2 wt % to 30 wt %, more preferably 5 wt. % to 20 wt. % of the total weight.

In other embodiments, the pharmaceutical compositions further comprise one or more additional materials such as a pharmaceutically compatible carrier, binder, viscosity modifier, filling agent, suspending agent, flavoring agent, sweetening agent, disintegrating agent, surfactant, preservative, lubricant, colorant, diluent, solubilizer, moistening agent, stabilizer, wetting agent, anti-adherent, parietal cell activator, anti-foaming agent, antioxidant, chelating agent, antifungal agent, antibacterial agent, or one or more combination thereof

### Continuous Drug Delivery

According to embodiments provided herein, a continuous delivery system comprises a formulation selected from the group consisting of a liquid formulation, a solid formulation, a semi-solid formulation, an emulsion formulation, a nanoparticle formulation, a matrix formulation, a film formulation, a patch formulation, an infusion pump, and/or combinations thereof. The embodiments are intended to be formulated to provide a continuous, sustained delivery to mitigate the peak and valley pharmacokinetic behavior associated with standard immediate release oral delivery forms. The embodiments are intended to be formulated to provide a route of administration selected from the group consisting of an oral, buccal, mucosal, rectal, transdermal, topical, parenteral, and or implantable, and or combinations thereof.

In an aspect, a method of providing continuous drug delivery systems and methods of a therapeutically effective amount of a pharmaceutical composition or formulation comprising active agent, for example, THC and/or CBD is provided. In some embodiments, the continuous drug delivery systems and methods delivers THC and/or CBD continuously at a predetermined hourly rate. In some embodiments, the predetermined hourly rate may range from 16 to 1400 μg/hour for example from 30 μg to 750 μg/hour, from 30 μg to 145 μg/hour, from 70 μg to 285 μg/hour, or from 185 μg to 725 μg/hour, such as 35 μg/hour, 75 μg/hour, 90 μg/hour, 140 μg/hour, 180 μg/hour, 190 μg/hour, 275 μg/hour, 450 μg/hour, or 700 μg/hour, or is an hourly rate between any two of these recited rates (inclusive), e.g., between 35-140 μg/hour or 75-280 μg/hour or 190 to 700 μg/hour.

In some embodiments, the continuous drug delivery systems and methods delivers active agent continuously to achieve a steady state plasma level of the THC and/or CBD compounds in a range of 3-140 μg/L, such as 3.5-140 μg/L, 3-75 μg/L, 3.5-75 μg/L, 3.5-14 μg/L, 7.5-28 μg/L, 19-70 μg/L, 9 μg/L, 18 μg/L, or 45 μg/L.

In some embodiments, the continuous drug delivery systems and methods delivers the active agent continuously for a predetermined number of days. In some embodiments, the predetermined number of days is one day, two days, three days, four days, five days, six days, seven days, eight days, nine days, ten day, eleven days, twelve day, thirteen days, or fourteen days. In other embodiments, the predetermined number of days is between 1-14 days, 1-12 days, 1-10 days, 1-7 days, 1-5 days, 1-3 days, 2-14 days, 2-12 days, 2-10 days, 2-7 days, 2-5 days, 2-3 days, 3-14 days, 3-12 days, 3-10 days, 3-7 days, 3-5 days, 4-14 days, 4-10 days, 7-14 days or 7-10 days.

In some embodiments, the hourly rate is selected to achieve a plasma concentration comparable to the active agent plasma concentration provided by an oral dose between 0 to 24 hours, such as from about 1 to 24 hrs, further such as between about 5 and 24 hrs, and even further such as between about 5 hours and 23 hours or between about 10 hours and 16 hours after ingestion. The oral dose can be from 2.5 to 50 mg once daily, for example, 2.5 mg, 4.0 mg, 5 mg, 10 mg, 15 mg, 20 mg, 25 mg, 30 mg, or 50 mg of the THC and/or CBD compound once daily or once every two days. The phrase “comparable to plasma concentration of an oral dose between 0 and 24 hours” can be understood from the description of Examples 6-9. For example, the area of the time concentration curve (AUC) calculated by the trapezoidal rule for the interval 0-24 hours from a graph of plasma concentration versus time of an oral dose divided by the hours (24 hours) is the hourly blood concentration associated with the AUC. The hourly AUC that can be achieved by the hourly rate of the continuous delivery method described herein would produce similar drug exposure over time exhibited by the oral dose. Such hourly rate of the continuous delivery method described herein is the one which achieves a plasma concentration comparable to the 0 hour to 24 hours blood level of an oral dose. In another embodiment, the continuous delivery method described herein achieves an AUC comparable to the AUC from an oral dose of the same compound in the 0-24 hour post dosing period.

In some embodiments, the continuous drug delivery systems and methods delivers the active agent continuously in a manner where the AUC thereof is between 10-60% of the exposure obtained from a standard of care treatment. The standard of care treatment can be intraperitoneal injection of, for example, 500 mcg once per day, or can be from 2.5 to 50 mg oral once daily, such as 2.5 mg, 4.0 mg, 5 mg, 10 mg, 15 mg, 20 mg, 25 mg, 30 mg, or 50 mg of the CBD and/or THC compound once daily or once every two days via oral administration. In another embodiment, the continuous drug delivery systems and methods herein provides for continuous administration of the active agent to provide an AUC that is between about 10 and 60% of the AUC provided by a standard of care treatment. In some embodiments, the standard of care treatment is an oral dose of CBD and/or THC from 2.5 mg to 50 mg once daily, such as at 2.5 mg, 5 mg, 10 mg, 15 mg, 20 mg, or 25 mg or 50 mg once daily or once every two days.

In some embodiments, the continuous drug delivery systems and methods delivers the active agent compound (such as CBD and/or THC) continuously at a dose rate that provides a blood level (m/L) equivalent to the blood level at a time point from 10 hours to 16 hours obtained from daily oral dose of 2.5-50 mg once daily (e.g., 2.5 mg, 5 mg, 10 mg, or 25 mg of active agent once per day). In the most preferred embodiments, the method delivers the active agent compound continuously at a dose rate that provides a blood level equivalent to the blood level at 12 hours obtained from daily oral dose of 2.5 to 50 mg once daily (e.g. 5 mg, 10 mg, or 25 mg of active agent once per day).

In some embodiments, the continuous drug delivery systems and methods delivers the active agent compound (such as THC and/or CBD) continuously at a dose rate such that the daily dose of the method is 10-75%, such as 15-70%, 15-25%, 40-50%, 10-45%, 45%-70%, 60-70%, 15%, 16%, 17%, 18%, 19%, 20%, 21%, 22%, 23%, 24%, 25%, 26%, 27%, 28%, 29%, 30%, 40%, 41%, 42%, 43%, 44%, 45%, 46%, 47%, 48%, 49%, 50%, 60%, 61%, 62%, 63%, 64%, 65%, 66%, 67%, 68%, 69%, or 70% of the daily dose of a standard of care treatment. In some embodiments, the standard of care treatment is intraperitoneal injection of, for example, 500 mcg once daily. In some embodiments, the standard of care treatment is FDA-approved once daily oral dose of the active agent such as at 5 mg, 10 mg, or 25 mg oral once per day.

The continuous drug delivery systems and methods provides for continuous delivery of the active agent comprises a pharmaceutically acceptable carrier. The pharmaceutically acceptable carrier should be compatible with other ingredients of the formulation, if any, and not harmful for the subject's health. Where the continuous delivery comprise infusion, the formulation ingredients may be selected to facilitate the infusion of the formulation. Exemplary carriers for formulations for continuous delivery include, without limitation, water, carboxymethyl cellulose (CMC), Tween 80, dimethyl sulfoxide (DMSO), ethanol, 2-hydroxypropyl-β-cyclodextrin, dextrose, and PEG400.

When the formulation is in a liquid format, the active agent is present, in some embodiments, at a concentration of between about 0.01-20 mg/mL, 0.05-5 mg/mL, 0.05-3 mg/mL, 0.1-4 mg/mL, 0.1-2.0 mg/mL or between about 0.1-1 mg/mL.

In certain embodiments, continuous drug delivery systems and methods comprise transdermal system or topical formulation may be in form of a liquid or gel and may be incorporated in a transdermal patch. For example, without limitation, the transdermal formulation may include a polymer matrix, which may be adhesive or non-adhesive, e.g., without limitation a polyacrylic adhesive. Matrix patches include those with a single matrix layer, or multiple matrix layers.

Usage of the continuous drug delivery systems and methods such as transdermal and topical systems described herein, will have dosages that vary depending on the mode of administration, the particular condition to be treated and the effect desired. Dosage may be transdermal application once daily for 1 day, 2 days, 3, day, 4 days, 5, days, 6 days, 7 days, 8 days, 9 days, 10 days, 11 days, 12 days, 13 days, or 14 days, or longer. Alternatively, application may be several times a day for 1 day, 2 days, 3, day, 4 days, 5, days, 6 days, 7 days, 8 days, 9 days, 10 days, 11 days, 12 days, 13 days, or 14 days, or longer. Alternatively application may be once every day, every 2 days, every 3 days every 4 days, every 5 days, every 6 days, every 7 days, every 8 days, every 9 days, every 10 days, every 11 days, every 12 days, every 13 days, or every 14 days.

In some embodiments, the continuous drug delivery systems and methods such as transdermal or topical formulations provide for a predetermined rate of delivery of the active components of the transdermal patch over a predetermined time period. In some embodiments, the predetermined time period is 24 hours, 48 hours, 72 hours, 96 hours, 120 hours, 144 hours, 7 days, 8 to 13 days, two weeks, or 15 days. In some further embodiments, the predetermined rate is an essentially constant rate with a coefficient of variation of less than about 90%, 85% or 80% over a predetermined time period.

In yet further embodiments, the continuous drug delivery systems and methods such as transdermal or topical formulations provide a range of predetermined blood serum levels of the active components of the transdermal patches in a patient over a predetermined time. In some embodiments, the predetermined time period is 24 hours, 48 hours, 72 hours, 96 hours, 120 hours, 144 hours, 7 days, 8 to 13 days, two weeks, or 15 days.

In yet further embodiments, the continuous drug delivery systems and methods, such as transdermal or topical formulations provide a plasma concentration of the active components of the transdermal patches in a therapeutic range in a patient over a predetermined time. In some embodiments, the predetermined time period is 24 hours, 48 hours, 72 hours, 96 hours, 120 hours, 144 hours, 7 days, 8 to 13 days, two weeks, or 15 days.

In yet further embodiments, the continuous drug delivery systems and methods, such as transdermal or topical formulations described herein allow for reduced variability in dosage of the active components in a patient over a predetermined time. In some embodiments, the predetermined time period is 24 hours, 48 hours, 72 hours, 96 hours, 120 hours, 144 hours, 7 days, 8 to 13 days, two weeks, or 15 days.

In some embodiments, the continuous drug delivery systems and methods, such as transdermal or topical formulation provided herein may be administered in dosage regimens such as once in a day, once in two days, once in three days, once in four days, once in five days, once in six days, once in a week, once in 8 to about 13 days, once in two weeks, once in 15 days to about 30 days.

In yet further embodiments, a pharmacokinetic assessment is performed on a blood sample of a subject who has been treated using the transdermal delivery systems described herein. The transdermal formulations are adjusted in response to the pharmacokinetic assessment. For example, the dosage may be adjusted such that a smaller patch, larger patch, or multiple transdermal patches are applied to the subject, or a patch having a more or less of a dose of active ingredients may be applied. In some embodiments, the formulation will be available in various dosage strengths and patch sizes in order to achieve optimum therapeutic outcome based on the subject's requirements. In one embodiment, more than one transdermal system or topical formulation is applied to a subject, and in some embodiments, between 1-5, 1-4, 1-3, 1-2, 2-5, 2-4, 2-3 patches are applied, and in other embodiments, 1, 2, 3, 4, 5, or 6 patches are applied.

### Cancer

The disclosure provides a method for treating neoplasia in a patient in need of such treatment, comprising: administering to the patient continuous drug delivery systems and methods as disclosed herein comprising therapeutically effective amounts of, for example, tetrahydrocannabinol (THC), cannabidiol (CBD), and combinations thereof, as exemplified herein, optionally in further combination with, for example, a neoplasia treating agent. The term “neoplasia” as used herein refers also to tumors, proliferative diseases, malignancies and their metastases.

Cancer is an abnormal growth of cells which tend to proliferate in an uncontrolled way and, in some cases, to metastasize (spread). Cancer is not one disease. It is a group of more than 100 different and distinctive diseases. Cancer can involve any tissue of the body and have many different forms in each body area. Most cancers are named for the type of cell or organ in which they start. A tumor can be cancerous or benign. A benign tumor means the tumor can grow but does not spread. A cancerous tumor is malignant, meaning it can grow and spread to other parts of the body. If a cancer spreads (metastasizes), the new tumor bears the same name as the original (primary) tumor. The frequency of a particular cancer may depend on gender. While skin cancer is the most common type of malignancy for both men and women, the second most common type in men is prostate cancer and in women, breast cancer.

Examples for cancer diseases are Adenocarcinomas of the head and neck (including salivary glands and oral cavity), gastrointestinal tract (including pharynx, esophagus, stomach, small intestine, large intestine, anus), lung, liver (including hepatocellular carcinoma, cholangiocarcinoma, and mixed tumors), extrahepatic biliary tract and gallbladder, pancreas (including ductal and acinar types), genitourinary tracts (ovaries, fallopian tubes, endometrium, cervix, and vagina, ureters, urinary bladder, testicles, epididymis, prostate), and skin adnexa; squamous cell carcinomas of the head and neck (including salivary glands and oral cavity), gastrointestinal tract (including pharynx, esophagus, anus), lung, intrahepatic and extrahepatic biliary tree (including gallbladder), pancreas, genitourinary tracts (including endometrium, cervix, vagina, ureters, urinary bladder, testicles, epididymis, prostate), and skin adnexa; germ cell tumors (including malignant teratoma, embryonal carcinoma, struma ovarii, yolk sac tumor, seminoma, choriocarcinoma); sarcomas (including leiomyosarcomas, rhabdomyosarcomas, angiosarcomas, hemangioendotheliomas, liposarcomas, chondosarcomas, fibrosarcomas, Ewing sarcoma, malignant nerve sheathe tumors, alveolar soft part sarcomas, clear cell sarcomas, synovial sarcoma, osteosarcomas); malignancies of the central nervous system (including astrocytomas, oligodendroglioma, glioblastoma, GBM, medulloblastoma); salivary gland malignancies (including adenoid cystic carcinoma, adenosquamous carcinoma, clear cell carcinoma, cystadenocarcinoma, mucoepidermoid carcinoma); mixed type carcinomas (including hepatocellular-cholangiocarcinomas, carcinosarcomas, mixed adenoneurondocrine carcinomas, adenosquamous carcinomas); hepatocellular carcinoma; blastic malignancies (including hepatoblastoma, neuroblastoma, ganglioneuroblastoma, nephroblastoma); renal cell carcinomas; neuroendocrine carcinomas; thyroid carcinomas (including papillary, follicular, medullary, anaplastic carcinomas); parathyroid carcinomas, pituitary gland carcinomas, adrenal gland carcinomas (including adrenocortical carcinomas, pheochromocytoma), and combinations thereof. In some embodiments of the present disclosure, the cancer is endometrial cancer, breast cancer, ovarian cancer, cervical cancer, fallopian tube cancer, testicular cancer, primary peritoneal cancer, colon cancer, colorectal cancer, small intestine cancer, squamous cell carcinoma of the anogenital region, melanoma, renal cell carcinoma, lung cancer, non-small cell lung cancer, squamous cell carcinoma of the lung, stomach cancer, bladder cancer, gall bladder cancer, liver cancer, thyroid cancer, laryngeal cancer, salivary gland cancer, esophageal cancer, head and neck cancer, squamous cell carcinoma of the head and neck, prostate cancer, pancreatic cancer, mesothelioma, Merkel cell carcinoma, sarcoma, glioblastoma, GBM, and a hematological cancer, such as multiple myeloma, B-cell lymphoma, T-cell lymphoma, Hodgkin's lymphoma/primary mediastinal B-cell lymphoma, or chronic myelogenous leukemia.

The methods of the disclosure can be used to treat any type of cancer known in the art. Non-limiting examples of cancers to be treated by the methods of the present disclosure can include melanoma (e.g., metastatic malignant melanoma), renal cancer (e.g. clear cell carcinoma), prostate cancer (e.g. hormone refractory prostate adenocarcinoma), pancreatic adenocarcinoma, breast cancer, colon cancer, lung cancer (e.g. non-small cell lung cancer), esophageal cancer, squamous cell carcinoma, liver cancer, ovarian cancer, cervical cancer, thyroid cancer, glioblastoma, GBM, glioma, leukemia, lymphoma, mesothelioma, sarcoma and other neoplastic malignancies. Additionally, the invention includes refractory or recurrent malignancies whose growth may be inhibited using the methods of the invention. In some embodiments, a cancer to be treated by the methods of the present disclosure include, for example, carcinoma, squamous carcinoma (for example, cervical canal, eyelid, tunica conjunctiva, vagina, lung, oral cavity, skin, urinary bladder, head and neck, tongue, larynx, and gullet), and adenocarcinoma (for example, prostate, small intestine, endometrium, cervical canal, large intestine, lung, pancreas, gullet, rectum, uterus, stomach, mammary gland, and ovary). In some embodiments, a cancer to be treated by the methods of the present disclosure further include sarcomata (for example, myogenic sarcoma), leukosis, neuroma, melanoma, and lymphoma.

In some embodiments, a patient or population of patients to be treated with combination therapy of the present disclosure have a solid tumor. In some embodiments, a solid tumor is a melanoma, renal cell carcinoma, lung cancer, bladder cancer, breast cancer, cervical cancer, colon cancer, gall bladder cancer, laryngeal cancer, liver cancer, thyroid cancer, stomach cancer, salivary gland cancer, prostate cancer, pancreatic cancer, mesothelioma, sarcoma or Merkel cell carcinoma. In some embodiments, a patient or population of patients to be treated with combination therapy of the present disclosure have a hematological cancer. In some embodiments, the patient has a hematological cancer such as diffuse large B cell lymphoma (“DLBCL”), Hodgkin's lymphoma (“HL”), Non-Hodgkin's lymphoma (“NHL”), Follicular lymphoma (“FL”), acute myeloid leukemia (“AML”), or Multiple myeloma (“MM”).

### Neoplasia Therapies

Any therapy (e.g., therapeutic or prophylactic agent) which is useful, has been used, is currently being used, or may be used for the prevention, treatment and/or management of neoplasia can be used to prevent, treat, and/or manage the patient whose neoplasia and/or cancer in accordance with the methods, compositions, and combinations of the disclosure. Also, neoplasia and/or cancer monitoring can be employed in conjunction with any therapy for cancer according to the instant invention. Therapies (e.g., therapeutic or prophylactic agents) include, but are not limited to, peptides, polypeptides, fusion proteins, nucleic acid molecules, small molecules, mimetic agents, synthetic drugs, inorganic molecules, and organic molecules. Non-limiting examples of cancer therapies include chemotherapies, radiation therapies, hormonal therapies, anti-angiogenesis therapies, targeted therapies, and/or biological therapies including immunotherapies and surgery. In certain embodiments, a prophylactically and/or therapeutically effective regimen comprises the administration of a combination of therapies.

Examples of neoplasia agents or therapies include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthracyclin; anthramycin; asparaginase; asperlin; azacitidine (Vidaza); azetepa; azotomycin; batimastat; benzodepa; bicalutamide; bisantrene hydrochloride; bisnafide dimesylate; bisphosphonates (e.g., pamidronate (Aredria), sodium clondronate (Bonefos), zoledronic acid (Zometa), alendronate (Fosamax), etidronate, ibandornate, cimadronate, risedromate, and tiludromate); bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine (Ara-C); dacarbazine; dactinomycin; daunorubicin hydrochloride; decitabine (Dacogen); demethylation agents, dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; EphA2 inhibitors; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; histone deacetylase inhibitors (HDAC-Is) hydroxyurea; idarubicin hydrochloride; ifosfamide; ilmofosine; imatinib mesylate (Gleevec, Glivec); interleukin II (including recombinant interleukin II, or rIL2), interferon alpha-2a; interferon alpha-2b; interferon alpha-n1; interferon alpha-n3; interferon beta-I a; interferon gamma-I b; iproplatin; irinotecan hydrochloride; lanreotide acetate; lenalidomide (Revlimid); letrozole; leuprolide acetate; liarozole hydrochloride; lometrexol sodium; lomustine; losoxantrone hydrochloride; masoprocol; maytansine; mechlorethamine hydrochloride; anti-CD2 antibodies (e.g., siplizumab (MedImmune Inc.; International Publication No. WO 02/098370, which is incorporated herein by reference in its entirety)); megestrol acetate; melengestrol acetate; melphalan; menogaril; mercaptopurine; methotrexate; methotrexate sodium; metoprine; meturedepa; mifepristone; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxaliplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; RU486; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Other examples of cancer therapies include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; anti estrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; ci s-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, dioxamycin; diphenyl spiromustine; docetaxel; docosanol; dolasetron; doxifluridine; droloxifene; dronabinol; duocarmycin SA; ebselen; ecomustine; edelfosine; edrecolomab; eflomithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; HMG CoA reductase inhibitors (e.g., atorvastatin, cerivastatin, fluvastatin, lescol, lupitor, lovastatin, rosuvastatin, and simvastatin); hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuproli de+estrogen+progesterone; leuprorelin; levamisole; LFA-3 TIP (Biogen, Cambridge, Mass.; International Publication No. WO 93/0686 and U.S. Pat. No. 6,162,432); liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B 1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; gamma secretase inhibitors, single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; 5-fluorouracil; leucovorin; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; thalidomide; velaresol; veramine; verdins; verteporfin; vinorelbine; vinxaltine; anti-integrin antibodies (e.g., anti-integrin a.sub.vb.sub.3 antibodies); vorozole; zanoterone; zeniplatin; zilascorb; and zinostatin stimalamer.

A non-limiting list of compounds that could be used to target neoplasia and/or cancer includes: inhibitors of interleukin-3 receptor (IL-3R) and CD 123 (including peptides, peptide-conjugates, antibodies, antibody-conjugates, antibody fragments, and antibody fragment-conjugates that target IL-3R or CD123); cantharidin; norcantharidin and analogs and derivatives thereof; Notch pathway inhibitors including gamma secretase inhibitors; sonic hedgehog/smoothened pathway inhibitors including cyclopamine and analogs thereof; antibodies to CD96; certain NF-kB/proteasome inhibitors including parthenolide and analogs thereof; certain triterpenes including celastrol; certain mTOR inhibitors; compounds and antibodies that target the urokinase receptor; sinefungin; certain inosine monophosphate dehydrogenase (IMPDH) inhibitors; PPAR-alpha and PPAR-gamma agonists and antagonists (including pioglitazone, tesaslitazar, muraglitazar, peliglitazar, lobeglitazone, balaglitazone, ragaglitazar, rosiglitazone, farglitazar, sodelglitazar, reglitazar, naveglitazar, oxeglitazar, metaglidasen, netoglitazone, darglitazone, englitazone, thiazolidinediones, aleglitazar, edaglitazone, rivoglitazone, troglitazone, imiglitazar, and sipoglitazar); telomerase inhibitors; antibodies to EpCAM (ESA); GSK-3 beta agonists and antagonists (including Lithium, 6-bromoinirubin-3′-oxime (BIO), TDZD8); Wnt pathway inhibitors including antibodies to frizzled or small molecules that inhibit disheveled/frizzled or beta catenin; anti-CD20 antibodies and conjugates (e.g. Rituxan, Bexxar, Zevalin) for novel use in multiple myeloma or melanoma; anti-CD133 antibody; anti-CD44 antibody; antibodies to IL-4; certain differentiation agents such as versnarinone; compounds that target CD33 such as an antibody or betulinic acid; compounds that target lactadherin such as an antibody; small molecules or antibodies that target CXCR4 or SDF-1; small molecules or antibodies that target multi-drug resistance pumps; inhibitors of survivin; inhibitors of XIAP; small molecules that target Bcl-2; antibodies to CLL-1; and furin inhibitors (such as cucurbitacins).

An additional non-limiting list of compounds that could also be used to target cancer and/or cancer cells includes: i) antibodies, antibody fragments, and proteins that are either naked or conjugated to a therapeutic moiety that target certain cell surface targets on cancer cells, or 11) small molecules known in the art including ones that can be further optimized (e.g., via chemistry) or identified via a cancer cell-based screen (e.g., such as one that would determine whether a compound impairs proliferation or viability of a cancer cell through standard methods, the cell surface and intracellular targets including (not meant to be exhaustive) are: Rex 1 (Zfp42), CTGF, Activin A, Wnt, FGF-2, HIF-1, AP-2gamma, Bmi-1, nucleostemin, hiwi, Moz-TIF2, Nanog, beta-arrestin-2, Oct-4, Sox2, stella, GDF3, RUNX3, EBAF, TDGF-1, nodal, ZFPY, PTNE, Evi-1, Pax3, Mcl-1, c-kit, Lex-1, Zfx, lactadherin, aldehyde dehydrogenase, BCRP, telomerase, CD133, Bcl-2, CD26, Gremlin, and FoxC2.

In some embodiments, the therapy(ies) is an immunomodulatory agent. Non-limiting examples of immunomodulatory agents include proteinaceous agents such as cytokines, peptide mimetics, and antibodies (e.g., human, humanized, chimeric, monoclonal, polyclonal, Fvs, ScFvs, Fab or F(ab)2 fragments or epitope binding fragments), nucleic acid molecules (e.g., antisense nucleic acid molecules and triple helices), small molecules, organic compounds, and inorganic compounds. In particular, immunomodulatory agents include, but are not limited to, methotrexate, leflunomide, cyclophosphamide, cytoxan, Immuran, cyclosporine A, minocycline, azathioprine, antibiotics (e.g., FK506 (tacrolimus)), methylprednisolone (MP), corticosteroids, steroids, mycophenolate mofetil, rapamycin (sirolimus), mizoribine, deoxyspergualin, brequinar, malononitriloamides (e.g., leflunamide), T cell receptor modulators, cytokine receptor modulators, and modulators mast cell modulators. Other examples of immunomodulatory agents can be found, e.g., in U.S. Publication No. 2005/0002934 A1 at paragraphs 259-275 which is incorporated herein by reference in its entirety. In one embodiment, the immunomodulatory agent is a chemotherapeutic agent. In an alternative embodiment, the immunomodulatory agent is an immunomodulatory agent other than a chemotherapeutic agent. In some embodiments, the therapy(ies) used in accordance with the invention is not an immunomodulatory agent.

In some embodiments, the therapy(ies) is an anti-angiogenic agent. Non-limiting examples of anti-angiogenic agents include proteins, polypeptides, peptides, fusion proteins, antibodies (e.g., human, humanized, chimeric, monoclonal, polyclonal, Fvs, ScFvs, Fab fragments, F(ab2 fragments, and antigen-binding fragments thereof) such as antibodies that specifically bind to TNF-alpha, nucleic acid molecules (e.g., anti sense molecules or triple helices), organic molecules, inorganic molecules, and small molecules that reduce or inhibit angiogenesis.

In certain embodiments, the therapy(ies) is an alkylating agent, a nitrosourea, an antimetabolite, and anthracyclin, a topoisomerase II inhibitor, or a mitotic inhibitor. Alkylating agents include, but are not limited to, busulfan, cisplatin, carboplatin, chlorambucil, cyclophosphamide, ifosfamide, decarbazine, mechlorethamine, mephalen, and themozolomide. Nitrosoureas include but are not limited to carmustine (BCNU) and lomustine (CCNU). Antimetabolites include but are not limited to 5-fluorouracil, capecitabine, methotrexate, gemcitabine, cytarabine, and fludarabine. Anthracyclins include but are not limited to daunorubicin, doxorubicin, epirubicin, idarubicin, and mitoxantrone. Topoisomerase II inhibitors include, but are not limited to, topotecan, irinotecan, etopiside (VP-16), and teniposide. Mitotic inhibitors include, but are not limited to taxanes (paclitaxel, docetaxel), and the vinca alkaloids (vinblastine, vincristine, and vinorelbine). In some embodiments of the invention, the therapy(ies) includes the administration cantharidin or an analog thereof. The invention includes the use of agents that target cancer cells. In certain embodiments, the agent acts alone. In other embodiments, the agent is attached directly or indirectly to another therapeutic moiety. Non-limiting examples of therapeutic moieties include, but are not limited to alkylating agents, anti-metabolites, plant alkaloids, cytotoxic agents, chemotherapeutic agents (e.g., a steroid, cytosine arabinoside, fluoruracil, methotrexate, aminopterin, mitomycin C, demecolcine, etoposide, mithramycin, calicheamicin, CC-1065, chlorambucil or melphalan), radionuclides, therapeutic enzymes, cytokines, toxins including plant-derived toxins, fungus-derived toxins, bacteria-derived toxin (e.g., deglycosylated ricin A chain, a ribosome inactivating protein, alpha-sarcin, aspergillin, restirictocin, a ribonuclease, a diphtheria toxin, Pseudomonas exotoxin, a bacterial endotoxin or the lipid A moiety of a bacterial endotoxin), growth modulators and RNase. In some embodiments, the agent used is an agent that binds to a marker, e.g., an antigen on a cancer cell. In a specific embodiment, the agent binds to an antigen that is expressed at a greater level on cancer cells than on normal cells. In a specific embodiment, the agent binds specifically to a cancer cell antigen that is not a normal cell. In other embodiments, the therapy(ies) is an agent that binds to a marker on cancer cells. In one embodiment, the agent that binds to a marker on cancer cells is an antibody or an antibody conjugated to a therapeutic moiety, or an antibody fragment conjugated to a therapeutic moiety.

For example, in a specific embodiment, the agent binds specifically to the IL-3 Receptor (IL-3R). In some embodiments, the agent that binds to the IL-3R is an antibody or an antibody fragment that is specific for IL-3R. In some embodiments, the antibody or antibody fragment is conjugated either chemically or via recombinant technology to a therapeutic moiety (e.g., a chemotherapeutic agent, a plant-, fungus- or bacteria-derived toxin, a radionuclide) using a linking agent to effect a cell killing response. In certain embodiments, the antibody, antibody-conjugate, antibody fragment, or antibody fragment-conjugate binds to the alpha-subunit of IL-3R (i.e., the CD 123 antigen). In other embodiments, the antibody, antibody-conjugate, antibody fragment, or antibody fragment-conjugate binds to the IL-3R, containing both the alpha and beta subunits. Methods for preparing antibodies to IL-3R and mimetics of antibodies to IL-3R are described in U.S. Pat. No. 6,733,743 B2, which is incorporated herein by reference in its entirety.

In other embodiments, the agent that binds to a marker on cancer cells is a ligand. In some embodiments, the ligand is a cytokine that binds to a cytokine receptor on cancer cells. In a particular embodiment, the ligand is interleukin-3 (IL-3) which can be conjugated to a therapeutic moiety that includes a chemotherapeutic agent, a plant-, fungus-, or bacteria-derived toxin, or a radionuclide. The IL-3-conjugate prophylactic and/or therapeutic therapy or regimen can be in the form of a recombinant fusion protein in embodiments where the conjugate is a toxin and the toxin is a protein, such as diphtheria toxin.

In certain embodiments, antibodies or fragments thereof that bind to a marker on cancer cells are substantially non-immunogenic in the treated subject. Methods for obtaining non-immunogenic antibodies include, but are not limited to, chimerizing the antibody, humanizing the antibody, and isolating antibodies from the same species as the subject receiving the therapy. Antibodies or fragments thereof that bind to markers in cancer cells can be produced using techniques known in the art.

In some embodiments, the therapy comprises the use of X-rays, gamma rays and other sources of radiation to destroy cancer cells and/or cancer cells. In specific embodiments, the radiation therapy is administered as external beam radiation or teletherapy, wherein the radiation is directed from a remote source. In other embodiments, the radiation therapy is administered as internal therapy or brachytherapy wherein a radioactive source is placed inside the body close to cancer cells, cancer cells and/or a tumor mass.

In some embodiments, the therapy used is a proliferation based therapy. Non-limiting examples of such therapies include a chemotherapy and radiation therapy as described supra.

Currently available therapies and their dosages, routes of administration and recommended usage are known in the art and have been described in such literature as the Physician's Desk Reference (60th ed., 2006).

When two prophylactically and/or therapeutically effective regimens are administered to a subject concurrently, the term “concurrently” is not limited to the administration of the cancer therapeutics at exactly the same time, but rather, it is meant that they are administered to a subject in a sequence and within a time interval such that they can act together (e.g., synergistically to provide an increased benefit than if they were administered otherwise). For example, the cancer therapeutics may be administered at the same time or sequentially in any order at different points in time; however, if not administered at the same time, they should be administered sufficiently close in time so as to provide the desired therapeutic effect, preferably in a synergistic fashion. The combination cancer therapeutics can be administered separately, in any appropriate form and by any suitable route. When the components of the combination cancer therapeutics are not administered in the same pharmaceutical composition, it is understood that they can be administered in any order to a subject in need thereof. For example, a first prophylactically and/or therapeutically effective regimen can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of the second cancer therapeutic, to a subject in need thereof. In various embodiments, the cancer therapeutics are administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart or no more than 48 hours apart. In one embodiment, the cancer therapeutics are administered within the same office visit. In another embodiment, the combination cancer therapeutics are administered at 1 minute to 24 hours apart.

In a specific embodiment, the combination therapies have the same mechanism of action. In another specific embodiment, the combination therapies each have a different mechanism of action.

### Indications

The disclosure provides continuous drug delivery systems and methods for the treatment and/or prevention and/or control of indications such as pain management, cancer, seizure disorders, Parkinson's Disease, symptoms associated with PD, and other indications using continuous drug delivery. Conditions suitable for treatment according to this disclosure include, for example, seizure disorders, pain syndromes, neurodegenerative diseases (including motor neuron diseases, myelopathies, radiculopathies, and disorders of the sympathetic nervous system), dementias, cerebrovascular conditions, movement disorders, brain trauma, cranial nerve disorders, neuropsychiatric disorders, and other disease neuropathies (including viral associated neuropathies, diabetes associated neuropathies, Guillian-Barre syndrome, dysproteinemias, transthyretin-induced neuropathies, and carpal tunnel syndrome). As used herein, seizure disorders include complex partial seizures, simple partial seizures, partial seizures with secondary generalization, generalized seizures (including absence, grand mal (tonic clonic), status epilepticus, tonic, atonic, myoclonic), neonatal and infantile spasms, drug-induced seizures, trauma-induced seizures, and febrile seizures, and additional specific epilepsy syndromes such as juvenile myoclonic epilepsy, Lennox-Gastaut, mesial temporal lobe epilepsy, nocturnal frontal lobe epilepsy, progressive epilepsy with mental retardation, and progressive myoclonic epilepsy, as well as seizures associated with CNS mass lesions.

Pain syndromes include, for example, headaches (e.g., migraine, tension, and cluster), acute pain, chronic pain, neuropathic pain, nociceptive pain, central pain and inflammatory pain, drug-induced neuropathic pain, causalgia, complex regional pain syndrome types I and II, and reflex sympathetic dystrophy (RSDS).

Neurodegenerative diseases include Alzheimer's disease, Parkinson's Disease, multiple sclerosis, Huntington's Disease, ALS, spinal muscular atrophy, muscular dystrophies prion-related diseases, cerebellar ataxia, Friedrich's ataxia, SCA, Wilson's disease, RP, Gullain Barre syndrome, Adrenoleukodystrophy, Menke's Sx, cerebral autosomal dominant arteriopathy with subcortical infarcts (CADASIL), Charcot Marie Tooth diseases, neurofibromatosis, von-Hippel Lindau, Fragile X, spastic paraplegia, tuberous sclerosis complex, Wardenburg syndrome, spinal motor atrophies, Tay-Sachs, Sandoff disease, familial spastic paraplegia, myelopathies, radiculopathies, encephalopathies associated with trauma, radiation, drugs and infection, and disorders of the sympathetic nervous system (e.g., Shy Drager (familial dysautonomia), diabetic neuropathy, drug-induced and alcoholic neuropathy).

Dementias include Alzheimer's disease, Parkinson's disease, Pick's disease, fronto-temporal dementia, vascular dementia, normal pressure hydrocephalus, Huntington's disease, and MCI.

Cerebrovascular conditions amenable to treatment according to the present disclosure include cerebrovascular disease and strokes (e.g., thrombotic, embolic, thromboembolic, hemorrhagic [including AVM and berry aneurysms], venoconstrictive, and venous).

Included in movement disorders are Parkinson's disease, dystonias, benign essential tremor, tardive dystonia, tardive dyskinesia, and Tourette's syndrome.

Brain trauma as used herein includes traumatic brain and spinal cord injuries as well as brain injuries from radiation.

Cranial nerve disorders include trigeminal neuropathy, trigeminal neuralgia, Menier's syndrome, glossopharangela neuralgia, dysphagia, dysphonia, cranial nerve palsies and Bell's palsy.

Neuropsychiatric disorders include panic syndrome, general anxiety disorder, phobic syndromes of all types, mania, manic depressive illness, hypomania, unipolar depression, depression, stress disorders, PTSD, somatoform disorders, personality disorders, psychosis, and schizophrenia), and drug dependence/addiction (e.g., alcohol, psychostimulants (e.g., crack, cocaine, speed, meth), opioids, and nicotine), and drug-induced psychiatric disorders.

Other disease neuropathies that may be treated with the compositions and methods described herein include Guillain-Barre, diabetes associated neuropathies, dysproteinemias, transthyretin-induced neuropathies, neuropathy associated with HIV, herpes viruses (including herpes zoster) or other viral infection, neuropathy associated with Lyme disease, carpal tunnel syndrome, tarsal tunnel syndrome, amyloid-induced neuropathies, leprous neuropathy, Bell's palsy, compression neuropathies, sarcoidosis-induced neuropathy, polyneuritis cranialis, heavy metal induced neuropathy, transition metal-induced neuropathy, drug-induced neuropathy, post-meningitis syndrome, post-polio syndrome, prion diseases, and radiation associated neuropathic syndromes.

Other diseases amenable to treatment with the present disclosure include fatigue syndromes (e.g., chronic fatigue syndrome and fibromyalgia), ataxic syndromes, olivopontoicerebellar degeneration, striatonigral degeneration, and axonic brain damage.

The present disclosure is particularly useful in the treatment of neuropsychiatric disorders such as depression, agitation, anxiety, seizure disorders such as grand mal seizures, status epilepticus, migraine pain treatment and prophylaxis, Alzheimer's disease, Parkinson's disease, and traumatic brain and spinal cord injury.

Also, the higher doses enabled by the present disclosure are expected to be of particular importance for dementias including Alzheimer's disease, Parkinson's disease, and vascular dementia, pain syndromes, including headaches and migraines, seizure disorders, movement disorders, and brain trauma.

Furthermore, the ease of use and convenience of a dosage form provided developed to be delivered at once per day or less frequent administration at a therapeutically effective quantity from the onset of therapy is of value in the treatment of dementias including Alzheimer's disease and Parkinson's disease, seizure disorders, pain syndromes, and cerebrovascular conditions.

Another aspect of the present disclosure is directed to the use of the continuous drug delivery systems and methods as disclosed herein as a therapeutic agent for the prophylaxis and/or treatment of immunoinflammatory disorder. The term “immunoinflammatory disorder” encompasses a variety of conditions, including autoimmune diseases, proliferative skin diseases, and inflammatory dermatoses. Immunoinflammatory disorders result in the destruction of healthy tissue by an inflammatory process, dysregulation of the immune system, and unwanted proliferation of cells. Examples of immunoinflammatory disorders are acne vulgaris; acute respiratory distress syndrome; Addison's disease; allergic rhinitis; allergic intraocular inflammatory diseases, antineutrophil cytoplasmic antibody (ANCA)-associated small-vessel vasculitis; ankylosing spondylitis; arthritis, asthma; atherosclerosis; atopic dermatitis; autoimmune hepatitis; autoimmune hemolytic anemia; autoimmune hepatitis; Behcet's disease; Bell's palsy; bullous pemphigoid; cerebral ischemia; chronic obstructive pulmonary disease; cirrhosis; Cogan's syndrome; contact dermatitis; COPD; Crohn's disease; Cushing's syndrome; dermatomyositis; diabetes mellitus; discoid lupus erythematosus; eosinophilic fasciitis; erythema nodosum; exfoliative dermatitis; fibromyalgia; focal glomerulosclerosis; focal segmental glomerulosclerosis; giant cell arteritis; gout; gouty arthritis; graft versus host disease; hand eczema; Henoch-Schonlein purpura; herpes gestationis; hirsutism; idiopathic cerato-scleritis; idiopathic pulmonary fibrosis; idiopathic thrombocytopenic purpura; immune thrombocytopenic purpura inflammatory bowel or gastrointestinal disorders, inflammatory dermatoses; lichen planus; lupus nephritis; lymphomatous tracheobronchitis; macular edema; multiple sclerosis; myasthenia gravis; myositis; nonspecific fibrosing lung disease; osteoarthritis; pancreatitis; pemphigoid gestationis; pemphigus vulgaris; periodontitis; polyarteritis nodosa; polymyalgia rheumatica; pruritus scroti; pruritis/inflammation, psoriasis; psoriatic arthritis; pulmonary histoplasmosis; rheumatoid arthritis; relapsing polychondritis; rosacea caused by sarcoidosis; rosacea caused by scleroderma; rosacea caused by Sweet's syndrome; rosacea caused by systemic lupus erythematosus; rosacea caused by urticaria; rosacea caused by zoster-associated pain; sarcoidosis; scleroderma; segmental glomerulosclerosis; septic shock syndrome; shoulder tendinitis or bursitis; Sjogren's syndrome; Still's disease; stroke-induced brain cell death; Sweet's disease; systemic lupus erythematosus; systemic sclerosis; Takayasu's arteritis; temporal arteritis; toxic epidermal necrolysis; transplant-rejection and transplant-rejection-related syndromes; tuberculosis; type-1 diabetes; ulcerative colitis; uveitis; vasculitis; and Wegener's granulomatosis.

As used herein, “non-dermal inflammatory disorders” include, for example, rheumatoid arthritis, inflammatory bowel disease, asthma, and chronic obstructive pulmonary disease. By “dermal inflammatory disorders” or “inflammatory dermatoses” is meant an inflammatory disorder selected from psoriasis, guttate psoriasis, inverse psoriasis, pustular psoriasis, erythrodermic psoriasis, acute febrile neutrophilic dermatosis, eczema, asteatotic eczema, dyshidrotic eczema, vesicular palmoplantar eczema, acne vulgaris, atopic dermatitis, contact dermatitis, allergic contact dermatitis, dermatomyositis, exfoliative dermatitis, hand eczema, pompholyx, rosacea, rosacea caused by sarcoidosis, rosacea caused by scleroderma, rosacea caused by Sweet's syndrome, rosacea caused by systemic lupus erythematosus, rosacea caused by urticaria, rosacea caused by zoster-associated pain, Sweet's disease, neutrophilic hidradenitis, sterile pustulosis, drug eruptions, seborrheic dermatitis, Pityriasis rosea, cutaneous kikuchi disease, pruritic urticarial papules and plaques of pregnancy, Stevens-Johnson syndrome and toxic epidermal necrolysis, tattoo reactions, Wells syndrome (eosinophilic cellulitis), reactive arthritis (Reiter's syndrome), bowel-associated dermatosis-arthritis syndrome, rheumatoid neutrophilic dermatosis, neutrophilic eccrine hidradenitis, neutrophilic dermatosis of the dorsal hands, balanitis circumscripta plasmacellularis, balanoposthitis, Behcet's disease, erythema annulare centrifugum, erythema dyschromicum perstans, erythema multiforme, granuloma annulare, hand dermatitis, lichen nitidus, lichen planus, lichen sclerosus et atrophicus, lichen simplex chronicus, lichen spinulosus, nummular dermatitis, pyoderma gangrenosum, sarcoidosis, subcorneal pustular dermatosis, urticaria, and transient acantholytic dermatosis.

By “proliferative skin disease” is meant a benign or malignant disease that is characterized by accelerated cell division in the epidermis or dermis. Examples of proliferative skin diseases are psoriasis, atopic dermatitis, nonspecific dermatitis, primary irritant contact dermatitis, allergic contact dermatitis, basal and squamous cell carcinomas of the skin, lamellar ichthyosis, epidermolytic hyperkeratosis, premalignant keratosis, acne, and seborrheic dermatitis. As will be appreciated by one skilled in the art, a particular disease, disorder, or condition may be characterized as being both a proliferative skin disease and an inflammatory dermatosis. An example of such a disease is psoriasis.

Symptoms and signs of inflammation associated with specific conditions include: rheumatoid arthritis: —pain, swelling, warmth and tenderness of the involved joints; generalized and morning stiffness; insulin-dependent diabetes mellitus-insulitis; this condition can lead to a variety of complications with an inflammatory component, including: —retinopathy, neuropathy, nephropathy; coronary artery disease, peripheral vascular disease, and cerebrovascular disease; autoimmune thyroiditis: —weakness, constipation, shortness of breath, puffiness of the face, hands and feet, peripheral edema, bradycardia; multiple sclerosis: —spasticity, blurry vision, vertigo, limb weakness, paresthesias; uveoretinitis: —decreased night vision, loss of peripheral vision; lupus erythematosus: —joint pain, rash, photosensitivity, fever, muscle pain, puffiness of the hands and feet, abnormal urinalysis (hematuria, cylinduria, proteinuria), glomerulonephritis, cognitive dysfunction, vessel thrombosis, pericarditis; scleroderma: —Raynaud's disease; swelling of the hands, arms, legs and face; skin thickening; pain, swelling and stiffness of the fingers and knees, gastrointestinal dysfunction, restrictive lung disease; pericarditis; renal failure; other arthritic conditions having an inflammatory component such as rheumatoid spondylitis, osteoarthritis, septic arthritis and polyarthritis: —fever, pain, swelling, tenderness; other inflammatory brain disorders, such as meningitis, Alzheimer's disease, AIDS dementia encephalitis: —photophobia, cognitive dysfunction, memory loss; other inflammatory eye inflammations, such as retinitis: —decreased visual acuity; inflammatory skin disorders, such as, eczema, other dermatites (e.g., atopic, contact), psoriasis, burns induced by UV radiation (sun rays and similar UV sources): —erythema, pain, scaling, swelling, tenderness; inflammatory bowel disease, such as Crohn's disease, ulcerative colitis: —pain, diarrhea, constipation, rectal bleeding, fever, arthritis; asthma: —shortness of breath, wheezing; other allergy disorders, such as allergic rhinitis: —sneezing, itching, runny nose conditions associated with acute trauma such as cerebral injury following stroke-sensory loss, motor loss, cognitive loss; heart tissue injury due to myocardial ischemia: —pain, shortness of breath; lung injury such as that which occurs in adult respiratory distress syndrome: —shortness of breath, hyperventilation, decreased oxygenation, pulmonary infiltrates; inflammation accompanying infection, such as sepsis, septic shock, toxic shock syndrome: —fever, respiratory failure, tachycardia, hypotension, leukocytosis; other inflammatory conditions associated with particular organs or tissues, such as: (i) nephritis (e.g., glomeralonephritis): —oliguria, abnormal urinalysis; (ii) inflamed appendix: —fever, pain, tenderness, leukocytosis; (iii) gout: —pain, tenderness, swelling and erythema of the involved joint, elevated serum and/or urinary uric acid; (iv) inflamed gall bladder: —abdominal pain and tenderness, fever, nausea, leukocytosis; (v) congestive heart failure: —shortness of breath, rales, peripheral edema; (vi) Type II diabetes: —end organ complications including cardiovascular, ocular, renal, and peripheral vascular disease; (vii) lung (pulmonary) fibrosis: —hyperventilation, shortness of breath, decreased oxygenation; (viii) vascular disease, such as atherosclerosis and restenosis: —pain, loss of sensation, diminished pulses, loss of function; and (ix) alloimmunity leading to transplant rejection: —pain, tenderness, fever.

Another aspect of the present disclosure is directed to the use of the continuous drug delivery system and methods as disclosed herein as a therapeutic agent for the prophylaxis and/or treatment of Neurodegenerative Disease.

The present disclosure also relates generally to the fields of neurology and psychiatry and to methods of protecting the cells of a mammalian central nervous system from damage or injury. Injuries or trauma of various kinds to the central nervous system (CNS) or the peripheral nervous system (PNS) can produce profound and long-lasting neurological and/or psychiatric symptoms and disorders. One form that this can take is the progressive death of neurons or other cells of the central nervous system (CNS), i.e., neurodegeneration or neuronal degeneration.

Neuronal degeneration as a result of, for example; Alzheimer's disease, multiple sclerosis, cerebral-vascular accidents (CVAs)/stroke, traumatic brain injury, spinal cord injuries, degeneration of the optic nerve, e.g., ischemic optic neuropathy or retinal degeneration and other central nervous system disorders is an enormous medical and public health problem by virtue of both its high incidence and the frequency of long-term sequelae. Animal studies and clinical trials have shown that amino acid transmitters (especially glutamate), oxidative stress and inflammatory reactions contribute strongly to cell death in these conditions. Upon injury or upon ischemic insult, damaged neurons release massive amounts of the neurotransmitter glutamate, which is excitotoxic to the surrounding neurons. Glutamate is a negatively charged amino acid that is an excitatory synaptic transmitter in the mammalian nervous system.

This nervous system injury may take the form of an abrupt insult or an acute injury to the nervous system as in, for example, acute neurodegenerative disorders including, but not limited to; acute injury, hypoxia-ischemia or the combination thereof resulting in neuronal cell death or compromise. Acute injury includes, but is not limited to, traumatic brain injury (TBI) including, closed, blunt or penetrating brain trauma, focal brain trauma, diffuse brain damage, spinal cord injury, intracranial or intravertebral lesions (including, but not limited to, contusion, penetration, shear, compression or laceration lesions of the spinal cord or whiplash shaken infant syndrome).

In addition, deprivation of oxygen or blood supply in general can cause acute injury as in hypoxia and/or ischemia including, but not limited to, cerebrovascular insufficiency, cerebral ischemia or cerebral infarction (including cerebral ischemia or infarctions originating from embolic occlusion and thrombosis, retinal ischemia (diabetic or otherwise), glaucoma, retinal degeneration, multiple sclerosis, toxic and ischemic optic neuropathy, reperfusion following acute ischemia, perinatal hypoxic-ischemic injury, cardiac arrest or intracranial hemorrhage of any type (including, but not limited to, epidural, subdural, subarachnoid or intracerebral hemorrhage).

Trauma or injury to tissues of the nervous system may also take the form of more chronic and progressive neurodegenerative disorders, such as those associated with progressive neuronal cell death or compromise over a period of time including, but not limited to, Alzheimer's disease, Pick's disease, diffuse Lewy body disease, progressive supranuclear palsy (Steel-Richardson syndrome), multisystem degeneration (Shy-Drager syndrome), chronic epileptic conditions associated with neurodegeneration, motor neuron diseases (amyotrophic lateral sclerosis), multiple sclerosis, degenerative ataxias, cortical basal degeneration, ALS-Parkinson's-dementia complex of Guam, subacute sclerosing panencephalitis, Huntington's disease, Parkinson's disease, synucleinopathies (including multiple system atrophy), primary progressive aphasia, striatonigral degeneration, Machado-Joseph disease or spinocerebellar ataxia type 3 and olivopontocerebellar degenerations, bulbar and pseudobulbar palsy, spinal and spinobulbar muscular atrophy (Kennedy's disease), primary lateral sclerosis, familial spastic paraplegia, Werdnig-Hoffmann disease, Kugelberg-Welander disease, Tay-Sach's disease, Sandhoff disease, familial spastic disease, Wohlfart-Kugelberg-Welander disease, spastic paraparesis, progressive multifocal leukoencephalopathy, familial dysautonomia (Riley-Day syndrome) or prion diseases (including, but not limited to Creutzfeld-Jakob disease, Gerstmann-Strussler-Scheinker disease, Kuru disease or fatal familial insomnia).

In addition, trauma and progressive injury to the nervous system can take place in various psychiatric disorders, including but not limited to, progressive, deteriorating forms of bipolar disorder or schizoaffective disorder or schizophrenia, impulse control disorders, obsessive compulsive disorder (OCD), behavioral changes in temporal lobe epilepsy and personality disorders.

In one preferred embodiment the continuous drug delivery systems and methods of the disclosure would be used to provide neuroprotection in disorders involving trauma and progressive injury to the nervous system in various psychiatric disorders. These disorders would be selected from the group consisting of; schizoaffective disorder, schizophrenia, impulse control disorders, obsessive compulsive disorder (OCD) and personality disorders.

In addition, trauma and injury make take the form of disorders associated with overt and extensive memory loss including, but not limited to, neurodegenerative disorders associated with age-related dementia, vascular dementia, diffuse white matter disease (Binswanger's disease), dementia of endocrine or metabolic origin, dementia of head trauma and diffuse brain damage, dementia pugilistica or frontal lobe dementia, including but not limited to Pick's Disease.

Other disorders associated with neuronal injury include, but are not limited to, disorders associated with chemical, toxic, infectious and radiation injury of the nervous system including the retina, injury during fetal development, prematurity at time of birth, anoxic-ischemia, injury from hepatic, glycemic, uremic, electrolyte and endocrine origin, injury of psychiatric origin (including, but not limited to, psychopathology, depression or anxiety), injury from peripheral diseases and plexopathies (including plexus palsies) or injury from neuropathy (including neuropathy selected from multifocal, sensory, motor, sensory-motor, autonomic, sensory-autonomic or demyelinating neuropathies (including, but not limited to Guillain-Barre syndrome or chronic inflammatory demyelinating polyradiculoneuropathy) or those neuropathies originating from infections, inflammation, immune disorders, drug abuse, pharmacological treatments, toxins, trauma (including, but not limited to compression, crush, laceration or segmentation traumas), metabolic disorders (including, but not limited to, endocrine or paraneoplastic), Charcot-Marie-Tooth disease (including, but not limited to, type 1a, 1b, 2, 4a or 1-X linked), Friedreich's ataxia, metachromatic leukodystrophy, Refsum's disease, adrenomyeloneuropathy, ataxia-telangiectasia, Djerine-Sottas (including, but not limited to, types A or B), Lambert-Eaton syndrome or disorders of the cranial nerves).

Further indications are cognitive disorders. The term “cognitive disorder” shall refer to anxiety disorders, delirium, dementia, amnestic disorders, dissociative disorders, eating disorders, mood disorders, schizophrenia, psychotic disorders, sexual and gender identity disorders, sleep disorders, somatoform disorders, acute stress disorder, obsessive-compulsive disorder, panic disorder, posttraumatic stress disorder, specific phobia, social phobia, substance withdrawal delirium, Alzheimer's disease, Creutzfeldt-Jakob disease, head trauma, Huntington's disease, HIV disease, Parkinson's disease, Pick's disease, learning disorders, motor skills disorders, developmental coordination disorder, communication disorders, phonological disorder, pervasive developmental disorders, Asperger's disorder, autistic disorder, childhood disintegrative disorder, Rett's disorder, pervasive developmental disorder, attention-deficit/hyperactivity disorder (ADHD), conduct disorder, oppositional defiant disorder, pica, rumination disorder, tic disorders, chronic motor or vocal tic disorder, Tourette's disorder, elimination disorders, encopresis, enuresis, selective mutism, separation anxiety disorder, dissociative amnesia, depersonalization disorder, dissociative fugue, dissociative identity disorder, anorexia nervosa, bulimia nervosa, bipolar disorders, schizophreniform disorder, schizoaffective disorder, delusional disorder, psychotic disorder, shared psychotic disorder, delusions, hallucinations, substance-induced psychotic disorder, orgasmic disorders, sexual pain disorders, dyspareunia, vaginismus, sexual dysfunction, paraphilias, dyssomnias, breathing-related sleep disorder, circadian rhythm sleep disorder, hypersomnia, insomnia, narcolepsy, dyssomnia, parasomnias, nightmare disorder, sleep terror disorder, sleepwalking disorder, parasomnia, body dysmorphic disorder, conversion disorder, hypochondriasis, pain disorder, somatization disorder, alcohol related disorders, amphetamine related disorders, caffeine related disorders, cannabis related disorders, cocaine related disorders, hallucinogen related disorders, inhalant related disorders, nicotine related disorders, opioid related disorders, phencyclidine-related disorder, abuse, persisting amnestic disorder, intoxication, withdrawal.

The term “bipolar and clinical disorders” shall refer to adjustment disorders, anxiety disorders, delirium, dementia, amnestic and other cognitive disorders, disorders usually first diagnosed in infancy (e.g.), childhood, or adolescence, dissociative disorders (e.g. dissociative amnesia, depersonalization disorder, dissociative fugue and dissociative identity disorder), eating disorders, factitious disorders, impulse-control disorders, mental disorders due to a general medical condition, mood disorders, other conditions that may be a focus of clinical attention, personality disorders, schizophrenia and other psychotic disorders, sexual and gender identity disorders, sleep disorders, somatoform disorders, substance-related disorders, generalized anxiety disorder (e.g. acute stress disorder, posttraumatic stress disorder), panic disorder, phobia, agoraphobia, obsessive-compulsive disorder, stress, acute stress disorder, anxiety neurosis, nervousness, phobia, posttraumatic stress disorder, posttraumatic stress disorder (PTSD), abuse, obsessive-compulsive disorder (OCD), manic depressive psychosis, specific phobias, social phobia, adjustment disorder with anxious features.

Examples for disorders usually first diagnosed in infancy, childhood, or adolescence are: mental retardation, learning disorders, mathematics disorder, reading disorder, disorder of written expression, motor skills disorders, developmental coordination disorder, communication disorders, expressive language disorder, phonological disorder, mixed receptive-expressive language disorder, stuttering, pervasive developmental disorders, Asperger's disorder, autistic disorder, childhood disintegrative disorder, Rett's disorder, pervasive developmental disorder, attention-deficit/hyperactivity disorder (ADHD), conduct disorder, oppositional defiant disorder, feeding disorder of infancy or early childhood, pica, rumination disorder, tic disorders, chronic motor or vocal tic disorder, Tourette's syndrome, elimination disorders, encopresis, enuresis, selective mutism, separation anxiety disorder, reactive attachment disorder of infancy or early childhood, stereotypic movement disorder.

Examples for substance-related disorders are: alcohol related disorders, amphetamine related disorders, caffeine related disorders, cannabis related disorders, cocaine related disorders, hallucinogen related disorders, inhalant related disorders, nicotine related disorders, opioid related disorders, psychotic disorder, psychotic disorder, phencyclidine-related disorder, abuse, persisting amnestic disorder, anxiety disorder, persisting dementia, dependence, intoxication, intoxication delirium, mood disorder, psychotic disorder, withdrawal, withdrawal delirium, sexual dysfunction, sleep disorder.

The term “neuroprotection” as used herein shall mean; inhibiting, preventing, ameliorating or reducing the severity of the dysfunction, degeneration or death of nerve cells, axons or their supporting cells in the central or peripheral nervous system of a mammal, including a human. This includes the treatment or prophylaxis of a neurodegenerative disease; protection against excitotoxicity or ameliorating the cytotoxic effect of a compound (for example, a excitatory amino acid such as glutamate; a toxin; or a prophylactic or therapeutic compound that exerts an immediate or delayed cytotoxic side effect including but not limited to the immediate or delayed induction of apoptosis) in a patient in need thereof.

The term “a patient in need of treatment with a neuroprotective drug” as used herein will refer to any patient who currently has or may develop any of the above syndromes or disorders, or any disorder in which the patient's present clinical condition or prognosis could benefit from providing neuroprotection to prevent the development, extension, worsening or increased resistance to treatment of any neurological or psychiatric disorder.

As used herein the term “combination administration” of a compound, therapeutic agent or known drug with the combination of the present disclosure means administration of the drug and the one or more compounds at such time that both the known drug and/or combination will have a therapeutic effect. In some cases this therapeutic effect will be synergistic. Such concomitant administration can involve concurrent (i.e. at the same time), prior, or subsequent administration of the drug with respect to the administration of the composition and/or combination of the present disclosure. A person of ordinary skill in the art would have no difficulty determining the appropriate timing, sequence and dosages of administration for particular drugs of the present disclosure.

Another aspect of the present disclosure is directed to the use of the continuous drug delivery systems and methods as disclosed herein as a therapeutic agent for the prophylaxis and/or treatment of heart disease. Heart disease is a general term used to describe many different heart conditions. For example, coronary artery disease, which is the most common heart disease, is characterized by constriction or narrowing of the arteries supplying the heart with oxygen-rich blood, and can lead to myocardial infarction, which is the death of a portion of the heart muscle. Heart failure is a condition resulting from the inability of the heart to pump an adequate amount of blood through the body. Heart failure is not a sudden, abrupt stop of heart activity but, rather, typically develops slowly over many years, as the heart gradually loses its ability to pump blood efficiently. Risk factors for heart failure include coronary artery disease, hypertension, valvular heart disease, cardiomyopathy, disease of the heart muscle, obesity, diabetes, and/or a family history of heart failure.

Examples of cardiovascular diseases and disorders are: aneurysm, stable angina, unstable angina, angina pectoris, angioneurotic edema, aortic valve stenosis, aortic aneurysm, arrhythmia, arrhythmogenic right ventricular dysplasia, arteriosclerosis, arteriovenous malformations, atrial fibrillation, Behcet syndrome, bradycardia, cardiac tamponade, cardiomegaly, congestive card iomyopathy, hypertrophic cardiomyopathy, restrictive cardiomyopathy, carotid stenosis, cerebral hemorrhage, Churg-Strauss syndrome, diabetes, Ebstein's Anomaly, Eisenmenger complex, cholesterol embolism, bacterial endocarditis, fibromuscular dysplasia, congenital heart defects, heart diseases, congestive heart failure, heart valve diseases, heart attack, epidural hematoma, hematoma, subdural, Hippel-Lindau disease, hyperemia, hypertension, pulmonary hypertension, cardiac hypertrophy, left ventricular hypertrophy, right ventricular hypertrophy, hypoplastic left heart syndrome, hypotension, intermittent claudication, ischemic heart disease, Klippel-Trenaunay-Weber syndrome, lateral medullary syndrome, long QT syndrome mitral valve prolapse, moyamoya disease, mucocutaneous lymph node syndrome, myocardial infarction, myocardial ischemia, myocarditis, pericarditis, peripheral vascular diseases, phlebitis, polyarteritis nodosa, pulmonary atresia, Raynaud disease, Sneddon syndrome, superior vena cava syndrome, syndrome X, tachycardia, Takayasu's arteritis, hereditary hemorrhagic telangiectasia, telangiectasis, temporal arteritis, tetralogy of Fallot, thromboangiitis obliterans, thrombosis, thromboembolism, tricuspid atresia, varicose veins, vascular diseases, vasculitis, vasospasm, ventricular fibrillation, Williams syndrome, peripheral vascular disease, varicose veins and leg ulcers, deep vein thrombosis, Wolff-Parkinson-White syndrome.

Vascular diseases are often the result of decreased perfusion in the vascular system or physical or biochemical injury to the blood vessel.

Peripheral vascular disease (PVD) is defined as a disease of blood vessels often encountered as narrowing of the vessels of the limbs. There are two main types of these disorders, functional disease which doesn't involve defects in the blood vessels but rather arises from stimuli such as cold, stress, or smoking, and organic disease which arises from structural defects in the vasculature such as atherosclerotic lesions, local inflammation, or traumatic injury. This can lead to occlusion of the vessel, aberrant blood flow, and ultimately to tissue ischemia.

One of the more clinically significant forms of PVD is peripheral artery disease (PAD). PAD is often treated by angioplasty and implantation of a stent or by artery bypass surgery. Clinical presentation depends on the location of the occluded vessel. For example, narrowing of the artery that supplies blood to the intestine can result in severe postprandial pain in the lower abdomen resulting from the inability of the occluded vessel to meet the increased oxygen demand arising from digestive and absorptive processes. In severe forms the ischemia can lead to intestinal necrosis. Similarly, PAD in the leg can lead to intermittent pain, usually in the calf, that comes and goes with activity. This disorder is known as intermittent claudication (IC) and can progress to persistent pain while resting, ischemic ulceration, and even amputation.

Peripheral vascular disease is also manifested in atherosclerotic stenosis of the renal artery, which can lead to renal ischemia and kidney dysfunction.

One disease in which vascular diseases and their complications are very common is diabetes mellitus. Diabetes mellitus causes a variety of physiological and anatomical irregularities, the most prominent of which is the inability of the body to utilize glucose normally, which results in hyperglycemia. Chronic diabetes can lead to complications of the vascular system which include atherosclerosis, abnormalities involving large and medium size blood vessels (macroangiopathy) and abnormalities involving small blood vessels (microangiopathy) such as arterioles and capillaries.

Patients with diabetes mellitus are at increased risk of developing one or more foot ulcers as a result of established long-term complications of the disease, which include impaired nerve function (neuropathy) and/or ischemia. Local tissue ischemia is a key contributing factor to diabetic foot ulceration.

In addition to large vessel disease, patients with diabetes suffer further threat to their skin perfusion in at least two additional ways. First, by involvement of the non-conduit arteries, which are detrimentally affected by the process of atherosclerosis, and secondly, and perhaps more importantly, by impairment of the microcirculatory control mechanisms (small vessel disease). Normally, when a body part suffers some form of trauma, the body part will, as part of the body's healing mechanism, experience an increased blood flow. When small vessel disease and ischemia are both present, as in the case of many diabetics, this natural increased blood flow response is significantly reduced. This fact, together with the tendency of diabetics to form blood clots (thrombosis) in the microcirculatory system during low levels of blood flow, is believed to be an important factor in ulcer pathogenesis.

As used herein, the term “peripheral vascular diseases” comprises any peripheral vascular disease including peripheral and autonomic neuropathies. Examples of “peripheral vascular disease” include peripheral arterial disease, such as chronic arterial occlusion including arteriosclerosis, arteriosclerosis obliterans and thromboangiitis obliterans (Buerger's disease), macroangiopathy, microangiopathy, diabetes mellitus, thrombophlebitis, phlebemphraxis, Raynaud's disease, Raynaud's syndrome, CREST syndrome, health hazard due to vibration, Sudeck's syndrome, intermittent claudication, cold sense in extremities, abnormal sensation in extremities, sensitivity to the cold, Meniere's disease, Meniere's syndrome, numbness, lack of sensation, anesthesia, resting pain, causalgia (burning pain), disturbance of peripheral circulation function, disturbance of nerve function, disturbance of motor function, motor paralysis, diabetic peripheral circulation disorder, lumbar spinal canal stenosis, diabetic neuropathy, shock, autoimmune disease such as erythematosis, rheumatoid disease and rheumatoid arthritis, autonomic neuropathy, diabetic autonomic neuropathy, autonomic imbalance, orthostatic hypotension, erectile dysfunction, female sexual dysfunction, retrograde ejaculation, cystopathy, neurogenic bladder, defective vaginal lubrication, exercise intolerance, cardiac denervation, heat intolerance, gustatory sweating, diabetic complication, hyperglycemia, hypoglycemia unawareness, hypoglycemia unresponsiveness; glaucoma, neovascular glaucoma, cataract, retinopathy, diabetic retinopathy, diabetic maculopathy, occlusion of retinal artery, obstruction of central artery of retina, occlusion of retinal vein, macular edema, aged macular degeneration, aged disciform macular degeneration, cystoid macular edema, palpebral edema, retinal edema, chorioretinopathy, neovascular maculopathy, uveitis, iritis, retinal vasculitis, endophthalmitis, panophthalmitis, metastatic ophthalmia, choroiditis, retinal pigment epithelitis, conjunctivitis, cyclitis, scleritis, episcleritis, optic neuritis, retrobulbar optic neuritis, keratitis, blepharitis, exudative retinal detachment, corneal ulcer, conjunctival ulcer, chronic nummular keratitis, Thygeson keratitis, progressive Mooren's ulcer, damage of skin, skin ulcer including foot ulcer, diabetic ulcer, burn ulcer, lower leg ulcer, postoperative ulcer, traumatic ulcer, ulcer after herpes zoster, radiation ulcer, drug induced ulcer, frostbite (cold injury), chilblain, gangrene and sudden gangrene, angina pectoris/variant angiitis, coronary arteriosclerosis (chronic ischemic heart disease, asymptomatic ischemic heart disease, arteriosclerotic cardiovascular disease), myocardial infarction, heart failure, congestive heart failure and painless ischemic heart disease, pulmonary edema, hypertension, pulmonary hypertension; portal hypertension, diabetic nephropathy, decubitus, renal failure.

### Additional Active Agents

As used herein the term “combination administration” of a compound, therapeutic agent or known drug with the continuous drug delivery systems and methods as disclosed herein in combination means administration of the drug and the one or more compounds at such time that both the known drug and/or combination will have a therapeutic effect. In some cases this therapeutic effect will be synergistic. Such concomitant administration can involve concurrent (i.e. at the same time), prior, or subsequent administration of the drug with respect to the administration of the composition and/or combination of the present invention. A person of ordinary skill in the art would have no difficulty determining the appropriate timing, sequence and dosages of administration for particular drugs of the present invention.

Further, active ingredient(s), where applicable, may be present either in the form of one substantially optically pure enantiomer or as a mixture of enantiomers or polymorphs thereof.

The active ingredient(s) may comprise one or more of the following therapeutic classes but not limited to adrenergic agent; adrenocortical steroid; adrenocortical suppressant; aldosterone antagonist; amino acid; anabolic; analeptic; analgesic; anesthetic; anorectic; anti-acne agent; anti-adrenergic; anti-allergic; anti-amebic; anti-anemic; anti-anginal; anti-arthritic; anti-asthmatic; anti-atherosclerotic; antibacterial; anticholinergic; anticoagulant; anticonvulsant; antidepressant; antidiabetic; anti diarrheal; antidiuretic; anti-emetic; anti-epileptic; antifibrinolytic; antifungal; antihemorrhagic; antihistamine; antihyperlipidemia; antihypertensive; antihypotensive; anti-infective; anti-inflammatory; antimicrobial; antimigraine; antimitotic; antimycotic, antinauseant, antineoplastic, antineutropenic, antiparasitic; antiproliferative; antipsychotic; antirheumatic; anti seborrheic; anti secretory; anti spasmodic; antithrombotic; anti-ulcerative; antiviral; appetite suppressant; blood glucose regulator; bone resorption inhibitor; bronchodilator; cardiovascular agent; cholinergic; depressant; diagnostic aid; diuretic; dopaminergic agent; estrogen receptor agonist; fibrinolytic; fluorescent agent; free oxygen radical scavenger; gastric acid suppressant; gastrointestinal motility effector; glucocorticoid; hair growth stimulant; hemostatic; histamine H2 receptor antagonists; hormone; hypocholesterolemic; hypoglycemic; hypolipidemic; hypotensive; imaging agent; immunizing agent; immunomodulator; immunoregulator; immunostimulant; immunosuppressant; keratolytic; LHRH agonist; mood regulator; mucolytic; mydriatic; nasal decongestant; neuromuscular blocking agent; neuroprotective; NMDA antagonist; non-hormonal sterol derivative; plasminogen activator; platelet activating factor antagonist; platelet aggregation inhibitor; psychotropic; radioactive agent; scabicide; sclerosing agent; sedative; sedative-hypnotic; selective adenosine A1 antagonist; serotonin antagonist; serotonin inhibitor; serotonin receptor antagonist; steroid; thyroid hormone; thyroid inhibitor; thyromimetic; tranquilizer; amyotrophic lateral sclerosis agent; cerebral ischemia agent; Paget's disease agent; unstable angina agent; vasoconstrictor; vasodilator; wound healing agent; xanthine oxidase inhibitor.

As indicated the continuous drug delivery systems and methods as disclosed herein may comprise auxiliary excipients such as for example diluents, binders, lubricants, surfactants, disintegrants, plasticizers, anti-tack agents, opacifying agents, pigments, and such like. As will be appreciated by those skilled in the art, the exact choice of excipient and their relative amounts will depend to some extent on the final oral dosage form.

### Parkinson's Disease

The disclosure provides continuous drug delivery systems and methods for the treatment and/or prevention and/or control of for example, Parkinson's disease (PD). PD is a degenerative disorder of the central nervous system mainly affecting the motor system and belongs to a group of conditions called motor system disorders, which are the result of the loss of dopamine-producing cells in the substantia nigra. The causes of this cell death are largely unknown. Early in the course of the disease, the most obvious symptoms are movement-related; these include shaking, rigidity, slowness of movement and difficulty with walking and gait. Later, thinking and behavioral problems may arise, with dementia commonly occurring in the advanced stages of the disease. Depression is the most common psychiatric symptom. Other symptoms include sensory, sleep and emotional problems. Parkinson's disease is more common in older people, with most cases occurring after the age of 50; when it is seen in young adults, it is called Young Onset PD (YOPD).

The four primary symptoms of PD are tremor, or trembling in hands, arms, legs, jaw, and face; rigidity, or stiffness of the limbs and trunk; bradykinesia, or slowness of movement; and postural instability, or impaired balance and coordination. As these symptoms become more pronounced, patients may have difficulty walking, talking, or completing other simple tasks. Other symptoms may include depression and other emotional changes; difficulty in swallowing, chewing, and speaking; urinary problems or constipation; skin problems; and sleep disruptions. There are currently no blood or laboratory tests that have been proven to help in diagnosing sporadic PD, and diagnosis is based on medical history and a neurological examination.

The main motor symptoms are collectively called parkinsonism, or a “parkinsonian syndrome.” The disease can be either primary or secondary. Primary Parkinson's disease is referred to as idiopathic (having no known cause), although some atypical cases have a genetic origin, while secondary parkinsonism is due to known causes like toxins. Many risks and protective factors have been investigated: the clearest evidence is for an increased risk of PD in people exposed to certain pesticides and a reduced risk in tobacco smokers. The pathology of the disease is characterized by the accumulation of a protein into Lewy bodies in neurons, and insufficient formation and activity of dopamine in certain parts of the midbrain. Where the Lewy bodies are located is often related to the expression and degree of the symptoms of an individual. Diagnosis of typical cases is mainly based on symptoms, with tests such as neuroimaging being used for confirmation.

Currently, there is no cure for PD, but a variety of medications provide relief from the symptoms. Treatments, typically the medications L-DOPA and dopamine agonists, improve the early symptoms of the disease. As the disease progresses and dopaminergic neurons continue to be lost, these drugs eventually become ineffective at treating the symptoms and at the same time produce a complication marked by involuntary writhing movements. Diet and some forms of rehabilitation have shown some effectiveness at improving symptoms. Surgery and deep brain stimulation have been used to reduce motor symptoms as a last resort in severe cases where drugs are ineffective. Research directions include investigations into new animal models of the disease and of the potential usefulness of gene therapy, stem cell transplants and neuroprotective agents. Medications to treat non-movement-related symptoms of PD, such as sleep disturbances and emotional problems, also exist.

Levodopa (L-DOPA) has been the most widely used treatment since 1967. L-DOPA is converted into dopamine in the dopaminergic neurons by the enzyme dopa decarboxylase. Since motor symptoms are produced by a lack of dopamine in the substantia nigra, the administration of L-DOPA temporarily diminishes the motor symptoms. Usually, affected individuals are given levodopa combined with carbidopa, which delays the conversion of levodopa into dopamine until it reaches the brain. Nerve cells can use levodopa to make dopamine and replenish the brain's dwindling supply. Although levodopa helps at least three-quarters of parkinsonian cases, not all symptoms respond equally to the drug. Bradykinesia and rigidity respond best, while tremor may be only marginally reduced. Problems with balance and other symptoms may not be alleviated at all. Anticholinergics may help control tremor and rigidity. Other drugs, such as bromocriptine, pramipexole, and ropinirole, mimic the role of dopamine in the brain, causing the neurons to react as they would to dopamine. An antiviral drug, amantadine, also appears to reduce symptoms.

An estimated 53 million people have PD resulting in about 103,000 deaths globally. Parkinson's disease typically occurs in people over the age of 60, with males being affected more often than females. The average life expectancy following diagnosis is between 7 and 14 years. Parkinson's disease is typically idiopathic (having no specific known cause); however, a proportion of cases can be attributed to known genetic factors. For example, mutations in specific genes have been shown to cause PD (e.g., alpha-synuclein (SNCA), parkin (PRKN), leucine-rich repeat kinase 2 (LRRK2), PTEN-induced putative kinase 1 (PINK1), DJ-1 and ATP13A2). Mutations in LRRK2 are the most common known cause of familial and sporadic PD, accounting for approximately 5% of individuals with a family history of the disease and 3% of sporadic cases. The LRRK2 G2019S gain of function gene mutation is one of the most prevalent mutations contributing to PD pathogenesis. While treatments for PD are available, more effective therapies are needed.

Modern treatments try to manage the early motor symptoms of the disease, mainly through the use of levodopa and dopamine agonists. As the disease progresses and dopamine neurons continue to be lost, a point eventually arrives at which these drugs become ineffective at treating the symptoms and at the same time produce a complication called dyskinesia, marked by involuntary writhing movements. Therefore, there is a need in the art to be able to deliver large oral equivalent dose of, for example, CBD to obtain a therapeutic effect, and eliminate or reduce discontinuance, adverse events, and side effects, such as diarrhea, somnolence, abdominal pain, weight gain, headaches, etc., as adverse events like liver enzyme elevations, associated with large oral doses to treat motor symptoms in subjects with Parkinson's Disease, including symptoms of Parkinson's Disease as well as symptoms indirectly associated with Parkinson's Disease, such as those arising as side effects of treatment.

The present disclosure provides continuous drug delivery systems and methods for the treatment and/or prevention and/or control of Parkinson's Disease in a patient comprising administration of, for example, CBD and/or THC, as set forth herein.

### Seizure Disorders

The present disclosure provides continuous drug delivery systems and methods for the treatment and/or prevention and/or control of seizure disorders in a patient comprising administration of, for example, CBD and/or THC, as set forth herein. A seizure is the physical findings or changes in behavior that occur after an episode of abnormal electrical activity in the brain. The term “seizure” is often used interchangeably with “convulsion.” Convulsions are when a person's body shakes rapidly and uncontrollably. During convulsions, the person's muscles contract and relax repeatedly.

Based on the type of behavior and brain activity, seizures are divided into two broad categories: generalized and partial (also called local or focal). Classifying the type of seizure helps doctors diagnose whether or not a patient has epilepsy.

Generalized seizures are produced by electrical impulses from throughout the entire brain, whereas partial seizures are produced (at least initially) by electrical impulses in a relatively small part of the brain. The part of the brain generating the seizures is sometimes called the focus.

There are six types of generalized seizures. The most common and dramatic, and therefore the most well-known, is the generalized convulsion, also called the grand-mal seizure. In this type of seizure, the patient loses consciousness and usually collapses. The loss of consciousness is followed by generalized body stiffening (called the “tonic” phase of the seizure) for 30 to 60 seconds, then by violent jerking (the “clonic” phase) for 30 to 60 seconds, after which the patient goes into a deep sleep (the “postictal” or after-seizure phase). During grand-mal seizures, injuries and accidents may occur, such as tongue biting and urinary incontinence.

Absence seizures cause a short loss of consciousness (just a few seconds) with few or no symptoms. The patient, most often a child, typically interrupts an activity and stares blankly. These seizures begin and end abruptly and may occur several times a day. Patients are usually not aware that they are having a seizure, except that they may be aware of “losing time.” Myoclonic seizures consist of sporadic jerks, usually on both sides of the body. Patients sometimes describe the jerks as brief electrical shocks. When violent, these seizures may result in dropping or involuntarily throwing objects. Clonic seizures are repetitive, rhythmic jerks that involve both sides of the body at the same time. Tonic seizures are characterized by stiffening of the muscles. Atonic seizures consist of a sudden and general loss of muscle tone, particularly in the arms and legs, which often results in a fall.

Seizures described herein can include epileptic seizures; acute repetitive seizures; cluster seizures; continuous seizures; unremitting seizures; prolonged seizures; recurrent seizures; status epilepticus seizures, e.g., refractory convulsive status epilepticus, non-convulsive status epilepticus seizures; refractory seizures; myoclonic seizures; tonic seizures; tonic-clonic seizures; simple partial seizures; complex partial seizures; secondarily generalized seizures; atypical absence seizures; absence seizures; atonic seizures; benign Rolandic seizures; febrile seizures; emotional seizures; focal seizures; gelastic seizures; generalized onset seizures; infantile spasms; Jacksonian seizures; massive bilateral myoclonus seizures; multifocal seizures; neonatal onset seizures; nocturnal seizures; occipital lobe seizures; post traumatic seizures; subtle seizures; Sylvan seizures; visual reflex seizures; or withdrawal seizures.

### Epilepsy

The present disclosure provides continuous drug delivery systems and methods for the treatment and/or prevention and/or control of epilepsy in a patient comprising administration of, for example, CBD and/or THC, as set forth herein. Epilepsy is a brain disorder characterized by repeated seizures over time. Types of epilepsy can include, but are not limited to generalized epilepsy, e.g., childhood absence epilepsy, juvenile myoclonic epilepsy, epilepsy with grand-mal seizures on awakening, West syndrome, Lennox-Gastaut syndrome, Dravet syndrome, Tuberous Sclerosis Complex (TSC), Treatment-Resistant Epilepsy, Treatment Resistant Pediatric Epilepsy, partial epilepsy, e.g., temporal lobe epilepsy, frontal lobe epilepsy, benign focal epilepsy of childhood.

Status epilepticus (SE) can include, e.g., convulsive status epilepticus, e.g., early status epilepticus, established status epilepticus, refractory status epilepticus, super-refractory status epilepticus; non-convulsive status epilepticus, e.g., generalized status epilepticus, complex partial status epilepticus; generalized periodic epileptiform discharges; and periodic lateralized epileptiform discharges. Convulsive status epilepticus is characterized by the presence of convulsive status epileptic seizures, and can include early status epilepticus, established status epilepticus, refractory status epilepticus, super-refractory status epilepticus. Early status epilepticus is treated with a first line therapy. Established status epilepticus is characterized by status epileptic seizures which persist despite treatment with a first line therapy, and a second line therapy is administered. Refractory status epilepticus is characterized by status epileptic seizures which persist despite treatment with a first line and a second line therapy, and a general anesthetic is generally administered. Super refractory status epilepticus is characterized by status epileptic seizures which persist despite treatment with a first line therapy, a second line therapy, and a general anesthetic for 24 hours or more.

Non-convulsive status epilepticus can include, e.g., focal non-convulsive status epilepticus, e.g., complex partial non-convulsive status epilepticus, simple partial non-convulsive status epilepticus, subtle non-convulsive status epilepticus; generalized non-convulsive status epilepticus, e.g., late onset absence non-convulsive status epilepticus, atypical absence non-convulsive status epilepticus, or typical absence non-convulsive status epilepticus.

Compositions described herein can also be administered as a prophylactic to a subject having a CNS disorder e.g., a traumatic brain injury, status epilepticus, e.g., convulsive status epilepticus, e.g., early status epilepticus, established status epilepticus, refractory status epilepticus, super-refractory status epilepticus; non-convulsive status epilepticus, e.g., generalized status epilepticus, complex partial status epilepticus; generalized periodic epileptiform discharges; and periodic lateralized epileptiform discharges; prior to the onset of a seizure.

Lennox-Gastaut Syndrome (LGS) is a severe form of epilepsy that typically becomes apparent during infancy or early childhood. Onset of LGS is usually between 2-7 years with a peak onset between 3 to 5 years. Affected Children experience several different types of seizures most commonly atonic, tonic and atypical absence seizures.

In 2018, GW Pharmaceutical received FDA approval for its fast track designated drug “Epidiolex” (Cannabidiol) to treat two orphan conditions in children—LGS and Dravet syndrome (DS).

Epidiolex contains naturally derived cannabidiol from Sativex plant in oral solution form (100 mg/ml). According to the FDA Label, the recommended dose of Epidiolex described in Table 1.

TABLE 1
Recommended dose of Epidiolex for LGS and DS¹

| | Avg wt of | Avg wt of | | |
|---|---|---|---|---|
| Recommended | 2 years old | 7-year-old | | |
| Dose | Child (kg) | Child (kg) | Total Dose (2-7 yrs) | |
| 5 mg/kg/day | 12.25 | 22.7 Kg | 61.25-113.5 | mg/day |
| 10 mg/kg/day | (12-12.5 kg) | (22.4-22.9 Kg) | 122.5-227 | mg/day |
| 20 mg/kg/day | | | 245-454 | mg/day |

Epidiolex has mainly five dose dependent side effects: 1) Hepatocellular injury, 2) Somnolence and sedation, 3) Suicidal Behavior and Ideation, 4) Hypersensitivity Reaction and 5) withdrawal of antiepileptic drugs¹.

The discontinuation rate of Epidiolex is high due to hepatocellular injury and somnolence and sedation side effects. During clinical trial 1.3% patient taking 10 mg/kg/day and 5.9% patient taking 20 mg/kg/day Epidiolex discontinue due to hepatocellular injury. Furthermore, there were 0% patient taking dose of 10 mg/kg/day and 3% patient taking 20 mg/kg/day dropped out due to somnolence and sedation. Table 2 provides the dose dependent side effects due to Epidiolex¹.

TABLE 2
Dose dependent Adverse effects: (FDA)¹

| | EPIDIOLEX | | |
|---|---|---|---|
| | 10 mg/kg/day | 20 mg/kg/day | Placebo |
| | N = 75 | N = 238 | N = 227 |
| Adverse Reactions | % | % | % |

Hepatic Disorders

| | | | |
|---|---|---|---|
| Transaminases elevated | 8 | 16 | 3 |
| Hepatocellular Injury | 1 | 17 | 0 |

Gastrointestinal Disorders

| | | | |
|---|---|---|---|
| Decreased appetite | 16 | 22 | 5 |
| Diarrhea | 9 | 20 | 9 |
| Weight decreased | 3 | 5 | 1 |
| Gastroenteritis | 0 | 4 | 1 |
| Abdominal pain, discomfort | 3 | 3 | 1 |

Nervous System Disorders

| | | | |
|---|---|---|---|
| Somnolence | 23 | 25 | 8 |
| Sedation | 3 | 6 | 1 |
| Lethargy | 4 | 8 | 2 |
| Fatigue, malaise, asthenia | 11 | 12 | 4 |
| Insomnia, sleep disorder, | 11 | 5 | 4 |
| poor quality sleep | | | |
| Irritability, agitation | 9 | 5 | 2 |
| Aggression, anger | 3 | 5 | <1 |
| Drooling, salivary | 1 | 4 | <1 |
| hypersecretion | | | |
| Gait disturbance | 3 | 2 | <1 |

Infections

| | | | |
|---|---|---|---|
| Infection, all | 41 | 40 | 31 |
| Infection, viral | 7 | 11 | 6 |
| Pneumonia | 8 | 5 | 1 |
| Infection, fungal | 1 | 3 | 0 |
| Infection, other | 25 | 21 | 24 |

Other

| | | | |
|---|---|---|---|
| Rash | 7 | 13 | 3 |
| Hypoxia, respiratory failure | 3 | 3 | 1 |

These side effects are dose related and therefore it is required to monitor the patients and reduced the dose if needed.

Many orally delivered drugs irritate the gastrointestinal mucosa and a large number, including CBD, undergo extensive ‘first-pass’ inactivation by the liver. The compositions and methods of the disclosure are directed to drug delivery directly to the systemic circulation, and thus the compositions and methods of the disclosure overcome the problems gastrointestinal tract irritation and liver first pass metabolism because the active agent avoids being metabolized by the liver after absorption, and also avoids gastrointestinal irritation since it is not orally administered.

However, the continuous drug delivery systems and methods as disclose herein deliver drug molecule constantly at defined input rate. Instead of peaks and valley in plasma concentrations like in oral delivery, the continuous drug delivery systems and methods as disclosed herein maintain the average plasma concentration at predetermined constant input rate.

While there are patents available on cannabidiol, the shortcomings of these disclosures are overcome by the compounds and methods as disclosed herein. For example, U.S. Pat. No. 9,375,417 fails to provide any in-vitro or in-vivo data. U.S. Pat. No. 6,328,992 discloses reservoir and adhesive matrix patches, however, these examples contain mixture of cannabinoids (such as delta-8-THC, delta-9-THC, cannabidiol, and cannabinol) instead of cannabidiol only. The THC is a psychoactive agent and an addictive substance, so the utility is problematic.

In addition, U.S. Pat. No. 8,449,908 discloses delivery of the cumulative amount of 60600 ng in 48 hrs through the human cadaver skin. This amount represents the flux of 1925 ng/sqcm/hr. The patch area can be calculated using following equation.

$$In - Vitro Flux ( ug / sqcm / hr ) = ( Css ( ug / l ) * CL ( L / hr ) / Patch Area ( Sqcm )$$

$$Patch Area ( Sqcm ) = ( CSS ( ug / l ) * CL ( L / hr ) / In - Vitro Flux ( ug / sqcm / hr ) = ( 10 - 74.4 ) / 1.925 = 386 sqcm$$

In order to deliver 5 mg/kg/day cannabidiol, the patient has to apply formulation on 386 sqcm surface area. This is an impractical patch size for any transdermal drug delivery system (TDDS). Furthermore, the '908 patent discloses using receiving media PBS:PEG-400 (60:40). It is very well known that PEG-400 is permeability enhancer and by incorporating it in receiving media, the skin is damaged from the dermis side, this can also increase the permeation amount due to unviable skin samples.

### Pain

The present disclosure provides continuous drug delivery systems and methods for the treatment and/or prevention and/or control of pain in a patient comprising administration of, for example, CBD and/or THC, as set forth herein. As used herein the following terms have the following meanings.

The term “chronic pain” or “chronic pain states” as used herein, is defined as any pain lasting longer than, for example, about 6 to about 12 weeks.

The term “neuropathic pain” as used herein has its conventional meaning and here means a pain arising as a direct or indirect consequence of a lesion or disease affecting the somatosensory system (central and/or peripheral). Neuropathic pain as used herein, includes all types of neuropathic pain, such as peripheral neuropathy caused by diabetes type 1 or 2, induced by various noxious substances such as alcohol, due to various deficiencies such as vitamin B1, B6 and/or B12 deficiency, various intoxications, such as hypervitaminosis B6, hypothyroidism, chemotherapeutic compound (e.g., paclitaxel or other taxane derivative, vincristine or other vinca alkaloids, cisplatin or other platinum derivate), drug-induced neuropathy, compounds for the treatment of infectious diseases (e.g., streptomycin, didanosine or zalcitabine), or any other chemically toxic compound. Other peripheral neuropathies include the following: trigeminal neuralgia, post-herpetic neuralgia, intercostal neuralgia, entrapment neuropathy (e.g., carpal tunnel syndrome, tarsal tunnel syndrome, abdominal cutaneous nerve entrapment syndrome), small fiber neuropathy, hereditary motor and sensory neuropathies, chronic inflammatory demyelinating polyneuropathy, sciatic pain chronic idiopathic sensory neuropathy, infectious disease conditions such as post-polio syndrome, AIDS or HIV-associated, Lyme-associated, Sjogren-associated, lymphomatous neuropathy, myelomatous neuropathy, carcinomatous neuropathy, acute pan autonomic neuropathy, vasculitic/ischaemic neuropathy and other mono- and polyneuropathies. Furthermore, under the term “neuropathic pain” also the following is included: complex regional pain syndrome type I and II (reflex sympathetic dystrophy), central neuropathic pain (e.g., thalamic neuropathy, spinal cord injury neuropathy, post stroke pain, multiple sclerosis neuropathy, syringomyelia, spinal cord tumors), phantom limb pain, restless genital syndrome (pain), post-surgical scar pain including cardiac surgery and mastectomy.

The term “inflammatory pain” as used herein has its conventional meaning and here means a pain that arises from inflammation that may be caused but by not limited to trauma, burns, extreme cold, fractures, (osteo)arthritis, rheumatoid arthritis, chronic strains, surgery, infection and autoimmune diseases excessive stretching, infections and vasoconstriction. Multiple inflammatory mediators can directly affect nociceptors or may sensitize them to touch or movement, even some distance from the inflammatory field.

The term “musculoskeletal pain” as used herein has its conventional meaning and here means a pain that affects the muscles, ligaments, tendons, bones, joints and/or soft tissues that are part of the musculoskeletal system. Musculoskeletal pain as used herein, includes all types of pain due to damage of muscle tissue as a result of wear and tear of daily activities. Trauma to an area (jerking movements, auto accidents, falls, sport injuries, fractures, sprains, strains dislocations, and direct blows to the muscle) also can cause musculoskeletal pain. Other causes of musculoskeletal pain include postural strain, repetitive movements, overuse, and prolonged immobilization, misuse of muscles, fibromyalgia, lumbar pain, pain due to increased muscle tone, and tendinitis due to overuse.

The term “analgesic” or “analgesics” as used herein has its conventional meaning and here refers to compounds, agents, drugs or substances that reduce pain in its broadest context.

The term “co-analgesic” or “co-analgesics” as used herein has its conventional meaning and here refers to compounds, agents, drugs or substances whose primary indication is for a purpose other than pain relief, which compounds demonstrate analgesic activity.

The term “reinstating analgesic effects” as used herein has its regular scientific meaning and is here referring to the capability (of a compound or of a composition) of reinstating an analgesic effect of at least one analgesic compound or at least one co-analgesic compound, when decreasing analgesic effect occurs after repeated use of a topical formulation containing at least one analgesic or co-analgesic compound.

The term “effect booster” or “co-analgesic effect booster” or “therapeutic effect booster” or “booster effect” or “synergistic effect” as used herein has its conventional meaning and here means the enhancement of a therapeutic effect induced by a co-analgesic compound (“co-analgesic”) leading to 1) intensified therapeutic effects of an active pharmaceutical ingredient with the purpose of alleviating neuropathic pain, inflammatory pain, musculoskeletal pain, pain due to muscle spasms, and/or other chronic pain states, 2) a faster onset of pain relieving effect, 3) a longer duration of analgesia, and/or 4) reinstating analgesic effects, when decreasing analgesic effect occurs after repeated use of a topical pharmaceutical composition containing at least one analgesic compound (“analgesic”) or co-analgesic compound.

Pain results from noxious stimulation of nerve endings. Nociceptive pain is caused by noxious stimulation of nociceptors that transmit impulses over intact neural pathways to the spinal neurons and then to the brain. Peripheral neuropathic pain is pain due to damage of the nerve endings, mostly found in the skin, especially in the epidermis. These damaged nerve endings can generate impulses in the absence of stimulation, can be hypersensitive to normal stimulation, and/or can be triggered by remaining local inflammatory stimulation. Even a very small number of damaged and overactive small nerve fibers in the epidermis are sufficient to trigger peripheral neuropathic pain. Neuropathic pain can be debilitating and can reduce quality of life of patients considerably. This pain may persist for months or years beyond the apparent healing of any damaged tissues.

Neuropathic pain has a local inflammatory component that results in sensitization of nerve fibers. Other intact nerve fibers, such as nociceptors being present up in the stratum granulosum, innervating the same region can also be sensitized and participate in clinical symptoms of neuropathic pain (e.g., hyperalgesia). This results in a situation of local neurogenic inflammation resulting in many different clinical features such as burning, freezing, electric shock, itch, tingling, pins and needles, hyperalgesia and allodynia (pain resulting from a non-painful stimulus such as a light touch or stroke).

Peripheral nerve damage leads to enhanced transmitter release within the spinal cord and can lead to central sensitization. Increased peripheral input through primary afferents is critically involved in central sensitization and the maintenance of neuropathic pain. Peripherally acting drugs, such as lidocaine 5% medicated patches and capsaicin 8% patches, have demonstrated the ability to reduce pain in neuropathic pain syndromes. However, lidocaine patches are not easy to apply, especially on the toes and by elderly, because the patch has to be cut, and many elderly cannot reach their toes properly. Application of capsaicin creams and patches quite often induce intolerable side effects, such as an increase of burning sensation, and often the treatment has to be combined with a local anesthetic to neutralize this side effect.

In chronic pain in general, for instance, oral analgesics such as acetaminophen, nonsteroidal anti-inflammatory drugs (NSAIDs) and opioids are part of guidelines aimed at to reduce the pain. Chronic use of such oral analgesics however, can induce serious and mortal side effects and/or detrimental drug-drug interactions.

Topical painkiller pharmaceutical compositions are also explored to help patients suffering from chronic pain. Two most commonly used topical compounds in neuropathic pain are capsaicin (vanilloid receptor agonist and counter-irritant) and lidocaine (membrane stabilizer), and both have clear drawbacks.

Clearly, there remains a pressing and long felt need in the art of developing treatment options for chronic pain in general and neuropathic pain, in particular, for the development of a novel and effective pharmaceutical composition for use in the treatment of chronic pain, the composition having reduced side effects in the patient.

### Dosage Forms

The continuous drug delivery systems and methods as disclosed herein can comprise active agent in, for example, a liquid formulation. Liquid formulations can also be prepared by dissolving or suspending one or the combination of active substances in a conventional liquid vehicle acceptable for administration so as to provide the desired dosage in, for example, one to four teaspoonfuls.

Dosage forms can be administered to the patient on a regimen of, for example, one, two, three, four, five, six, or other multiple doses per day.

In order to more finely regulate the dosage schedule, the active substances may be administered separately in individual dosage units at the same time or carefully coordinated times. The respective substances can be individually formulated in separate unit dosage forms in a manner similar to that described above.

In formulating the compositions, the active substances, in the amounts described above, may be compounded according to accepted practice with a physiologically acceptable vehicle, carrier, excipient, binder, viscosity modifier, preservative, stabilizer, flavor, etc., in the particular type of unit dosage form.

### Packaging/Treatment Kits

The disclosure provides a kit for conveniently and effectively carrying out the methods in accordance with the present disclosure. Such kits may be suited for the delivery of solid oral forms such as tablets or capsules. Such a kit may include a number of unit dosages. Such kits can include a means for containing the dosages oriented in the order of their intended use. An example of a means for containing the dosages in the order of their intended uses is a card. An example of such a kit is a “blister pack”. Blister packs are well known in the packaging industry and are widely used for packaging unit dosage forms. If desired, the blister can be in the form of a childproof blister, i.e., a blister that is difficult for a child to open, yet can be readily opened by an adult. If desired, a memory aid can be provided, for example in the form of numbers, letters, or other markings or with a calendar feature and/or calendar insert, designating the days and the sections of a day in the treatment schedule in which the dosages can be administered, such as, for example, an AM dose is packaged with a “midday” and a PM dose; or an AM dose is packaged with a PM dose. Alternatively, placebo dosages, or vitamin or dietary supplements, either in a form similar to or distinct from the active dosages, can be included.

The disclosure provides compositions, including preparations, formulations and/or kits, comprising combinations of ingredients, as described above (including the multi-ingredient combinations of drugs), that are serviceable as therapies for treating, preventing or improving conditions, states and disease as provided in the invention. In one aspect, each member of the combination of ingredients is manufactured in a separate package, kit or container; or, all or a subset of the combinations of ingredients are manufactured in a separate package or container. In alternative aspects, the package, kit or container comprises a blister package, a clamshell, a tray, a shrink wrap and the like.

In one aspect, the package, kit or container comprises a “blister package” (also called a blister pack, or bubble pack). In one aspect, the blister package consists two or more separate compartments. This blister package is made up of two separate material elements: a transparent plastic cavity shaped to the product and its blister board backing. These two elements are then joined together with a heat sealing process which allows the product to be hung or displayed. Exemplary types of “blister packages” include: Face seal blister packages, gang run blister packages, mock blister packages, interactive blister packages, slide blister packages.

Blister packs, clamshells or trays are forms of packaging used for goods; thus, the invention provides for blister packs, clamshells or trays comprising a composition (e.g., a (the multi-ingredient combination of drugs of the invention) combination of active ingredients) of the invention. Blister packs, clamshells or trays can be designed to be non-reclosable, so consumers can tell if a package has already opened. They are used to package for sale goods where product tampering is a consideration, such as the agents of the invention. In one aspect, a blister pack of the invention comprises a moulded PVC base, with raised areas (the “blisters”) to contain the tablets, pills, etc. comprising the combinations of the invention, covered by a foil laminate. Tablets, pills, etc. are removed from the pack either by peeling the foil back or by pushing the blister to force the tablet to break the foil. In one aspect, a specialized form of a blister pack is a strip pack.

In one aspect, a blister pack also comprises a method of packaging where the compositions comprising combinations of ingredients of the invention are contained in-between a card and clear PVC. The PVC can be transparent so the item (pill, tablet, geltab, etc.) can be seen and examined easily; and in one aspect, can be vacuum-formed around a mould so it can contain the item snugly and have room to be opened upon purchase. In one aspect, the card is brightly colored and designed depending on the item (pill, tablet, geltab, etc.) inside, and the PVC is affixed to the card using pre-formed tabs where the adhesive is placed. The adhesive can be strong enough so that the pack may hang on a peg, but weak enough so that this way one can tear open the join and access the item. Sometimes with large items or multiple enclosed pills, tablets, geltabs, etc., the card has a perforated window for access. In one aspect, more secure blister packs, e.g., for items such as pills, tablets, geltabs, etc. of the invention are used, and they can comprise of two vacuum-formed PVC sheets meshed together at the edges, with the informative card inside.

In one aspect, blister packaging comprises at least two components (e.g., is a multi-ingredient combination of drugs of the invention): a thermoformed “blister” which houses the product (e.g., a combination of the invention), and then a “blister card” that is a printed card with an adhesive coating on the front surface. During the assembly process, the blister component, which is most commonly made out of PVC, is attached to the blister card using a blister machine. Conventional blister packs can also be sealed.

As discussed herein, the products of manufacture of the invention can comprise the packaging of the therapeutic drug combinations of the invention, alone or in combination, as “blister packages” or as a plurality of packettes, including as lidded blister packages, lidded blister or blister card or packets, or a shrink wrap.

In one aspect, any of the invention's products of manufacture, including kits or blister packs, include memory aids to help remind patients when and how to take the agents of the invention.

The treatment kits can be constructed in a variety of forms familiar to one of ordinary skill in the art. The kits comprise at least one unit dosage of an active for administration according to a daily regimen and a means for containing the unit dosages. The treatment kits can, for example, be constructed for administration once daily, twice daily, thrice daily, four times daily, multiple administrations daily, or other dosage regimens. The kits comprise a means for the daily administration of an agent of the invention. In one embodiment the kits include from about one to about four unit dosages.

In one embodiment, the means for containing the unit dosages is a card, including, for example, a card that is capable of being folded. This card will be referred to herein as a main card, or alternatively a principal card or a first card, to distinguish it from additional optional cards, circulars, or other such materials which can be associated with the kit. This main card can be folded with a simple crease, or alternatively, with a double crease, so as to exhibit a spine, similar to the spine of a closed book. The main card can comprise a printable surface, i.e. a surface upon which the product name, appropriate administration instructions, product information, drawings, logos, memory aids, calendar features, etc. can be printed. The main card can comprise a means for containing said unit dosage or different dosages designated for different time of the day, and a memory aid for administering said unit dosage or dosages. The main card, especially if it is prepared from two or more laminated paperboard surfaces, can comprise a slit or pocket, for example in one of the inner paperboard surfaces of the folded card. The slit or pocket can be used to contain a removable secondary card, i.e., a second card or insert card, which is not permanently attached or affixed to the main card.

The memory aid can include a listing of the days of the week, i.e. Sunday, Monday, Tuesday, Wednesday, Thursday, Friday, and Saturday, with appropriate spaces for the patient to select and indicate on the card the preferred day of the week on which to administer the therapy. The memory aid can include a listing of the time of day with appropriate spaces for the patient to select and indicate on the card the preferred time of day (e.g.: AM, PM, midday) at which to administer the therapy. The memory aid can also include removable stickers having an appropriate pressure sensitive adhesive to facilitate easy removal and refastening to a desired surface such as a calendar or dayminder. The removable stickers can be located on the main card, or can be located on the secondary card which is constructed so that it can be readily inserted into and removed from the optional slit in the main card. Additionally, the optional slit can contain additional patient information and other circulars.

Other means for containing said unit dosages can include bottles and vials, wherein the bottle or vial comprises a memory aid, such as a printed label for administering said unit dosage or dosages. The label can also contain removable reminder stickers for placement on a calendar or dayminder to further help the patient to remember when to take a dosage or when a dosage has been taken.

The invention will be illustrated in more detail with reference to the following Examples, but it should be understood that the present invention is not deemed to be limited thereto.

### EXAMPLES

### Example 1

The continuous drug delivery systems and methods as disclosed herein may comprise solvents known to those skilled in the art either alone or in combinations thereof without any limitation to following like alcohol C₁-C₂₀ such as but not limited to (methanol, ethanol, isopropyl alcohol, butanol, propanol etc.), polyhydric alcohols, glycols such as but not limited to (propylene glycol, polyethylene glycol, dipropylene glycol, hexylene glycol, butylene glycol, glycerine etc.), derivative of glycols, pyrrolidone such as but not limited to (N methyl 2-pyrrolidone, 2-pyrrolidone etc.), sulfoxides such as but not limited to (dimethyl sulfoxide, decymethylsulfoxide etc), dimethylisosorbide, mineral oils, vegetable oils, sesame oil water, polar solvents, semi polar solvents, non polar solvents, volatile chemicals which can be used to make matrix patch such as but not limited to (ethanol, propanol, ethyl acetate, acetone, methanol, dichloromethane, chloroform, toluene, IPA, hexane), acids such as but not limited to acetic acid, lactic acid, levulinic acid, bases and others, pentane, dimethylformamide, butane, lipids. More preferably in the range of 0.01%-95% w/w or w/v. In exemplary embodiments, formulations of the disclosure may comprise solvents at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, about 80%, about 95%, about 96%, about 97%, about 98%, and about 99% of the formulation. In exemplary embodiments, formulations of the disclosure may comprise solvents at a concentration of about 1 to 20%, of about 5% to 25%, about 10% to about 20%, or about 15% to about 18%, about 30% to about 70%, about 35% to about 65%, about 63.13%, and about 40% to about 64% w/w. In exemplary formulations of the disclosure, the solvents will represent approximately 1 wt % to 75 wt %, preferably 2 wt % to 30 wt %, more preferably 5 wt. % to 20 wt. % of the formulation.

### Example 2

The continuous drug delivery system and methods as disclosed herein may comprise gelling agents and/or thickening and/or suspending agents and/or polymers and/or adhesive polymers and/or pressure sensitive adhesive polymers known to those skilled in the art either alone or in combinations thereof without any limitation to following like natural polymers, polysaccharides and its derivatives such as but not limited to (agar, alginic acid and derivatives, cassia tora, collagen, gelatin, gellum gum, guar gum, pectin, potassium, or sodium carageenan, tragacanth, xantham, gum copal, chitosan, resin etc.), semisynthetic polymers and its derivatives such as without any limitation to cellulose and its derivatives (methylcellulose, ethyl cellulose, carboxymethyl cellulose, hydroxylpropyl cellulose, hydroxylpropylmethyl cellulose etc.), synthetic polymers and its derivatives such as without any limitation to carboxyvinyl polymers or carbomers (carbopol 940, carbopol 934, carbopol 9′71p NF), polyethylene, and its copolymers etc, clays such as but not limited to (silicates, bentonite), silicon dioxide, polyvinyl alcohol, acrylic polymers (eudragit), acrylic acid esters, polyacrylate copolymers, polyacrylamide, polyvinyl pyrrolidone homopolymer and polyvinyl pyrrolidone copolymers such as but not limited to (PVP, Kollidon 30, poloxamer), isobutylene, ethyl vinyl acetate copolymers, natural rubber, synthetic rubber, pressure sensitive adhesives such as silicone polymers such as but not limited to (bio psa 4302, bio-psa 4202 etc.), acrylic pressure sensitive adhesives such as but not limited to (duro-tak 87-2156, duro-tak 387-2287, duro-tak 87-9301, duro-tak 387-2051, etc.), polyisobutylene such as but not limited to (polyisobutylene low molecular weight, polyisobutylene medium molecular weight, polyisobutylene 35000 mw, etc), acrylic copolymers, rubber based adhesives, hot melt adhesives, styrene-butadiene copolymers, bentonite, all water and/or organic solvent swellable polymers, etc. In exemplary embodiments, formulations of the disclosure may comprise gelling agents and/or thickening and/or suspending agents and/or polymers and/or adhesive polymers and/or pressure sensitive adhesive polymers at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 80%, about 95%, about 96%, about 97%, about 98%, and about 99% of the formulation. In exemplary embodiments, formulations of the disclosure may comprise gelling agents and/or thickening and/or suspending agents and/or polymers and/or adhesive polymers and/or pressure sensitive adhesive polymers at a concentration of about 1 to 20%, of about 5% to 25%, about 10% to about 20%, or about 15% to about 18%, about 30% to about 70%, about 35% to about 65%, about 63.13%, and about 40% to about 64% w/w. In exemplary formulations of the disclosure, the gelling agents and/or thickening and/or suspending agents and/or polymers and/or adhesive polymers and/or pressure sensitive adhesive polymers will represent approximately 1 wt % to 75 wt %, preferably 2 wt % to 30 wt %, more preferably 5 wt. % to 20 wt. % of the formulation, more preferably in the range of 0.1% 80% w/w or w/v.

### Example 3

The continuous drug delivery systems and methods as disclosed herein may comprise permeation enhancers known to those skilled in the art either alone or in combination thereof without any limitation to the following, such as sulfoxides, and similar chemicals such as but not limited to (dimethylsulfoxide, dimethylacetamide, dimethylformamide, decymethylsulfoxide, dimethylisosorbide etc), azone, pyrrolidones such as but not limited to (N-methyl-2-pyrrolidone, 2-pyrrolidon etc.), esters, fatty acid esters such as but not limited to (propylene glycol monolaurate, butyl ethanoate, ethyl ethanoate, isopropyl myristate, isopropyl palmitate, methyl ethanoate, lauryl lactate, ethyl oleate, decyl oleate, glycerol monooleate, glycerol monolaurate, lauryl laurate etc.), fatty acids such as but not limited to (capric acid, caprylic acid, lauric acid, oleic acid, myristic acid, linoleic acid, stearic acid, palmitic acid etc.), alcohols, fatty alcohols and glycols such as but not limited to (oleyl alcohol, nathanol, dodecanol, propylene glycol, glycerol etc.), ethers alcohol such as but not limited to (diethylene glycol monoethyl ether), urea, triglycerides such as but not limited to triacetin, polyoxyethylene fatty alcohol ethers, polyoxyethylene fatty acid esters, esters of fatty alcohols, essential oils, surfactant type enhancers such as but not limited to (brij, sodium lauryl sulfate, tween, polysorbate), terpene, terpenoids and all penetration or permeation enhancers referred in the book “Percutaneous Penetration Enhancers” (Eric W. Smith, Howard I. Maibach, 2005. November, CRC press). In exemplary embodiments, formulations of the disclosure may comprise permeation enhancers at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 80%, about 95%, about 96%, about 97%, about 98%, and about 99% of the formulation. In exemplary embodiments, formulations of the disclosure may comprise permeation enhancers at a concentration of about 1 to 20%, of about 5% to 25%, about 10% to about 20%, or about 15% to about 18%, about 30% to about 70%, about 35% to about 65%, about 63.13%, and about 40% to about 64% w/w. In exemplary formulations of the disclosure, the permeation enhancers will represent approximately 1 wt % to 75 wt %, preferably 2 wt % to 30 wt %, more preferably 5 wt. % to 20 wt. % of the formulation, more preferably in the range of 0.01%-95% w/w or w/v.

### Example 4

The continuous drug delivery systems and methods as disclosed herein may comprise plasticizers known to those skilled in the art either alone or in combination thereof without any limitation to following like glycerol and its esters, phosphate esters, glycol derivatives, sugar alcohols, sebacic acid esters, citric acid esters, tartaric acid esters, adipate, phthalic acid esters, triacetin, oleic acid esters and all the plasticizers which can be used in to continuous drug delivery system referred in the book “Handbook of Plasticizers” (George Wypych, 2004, Chem Tec Publishing). In exemplary embodiments, formulations of the disclosure may comprise plasticizers at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, about 80%, about 95%, about 96%, about 97%, about 98%, and about 99% of the formulation. In exemplary embodiments, formulations of the disclosure may comprise plasticizers at a concentration of about 1 to 20%, of about 5% to 25%, about 10% to about 20%, or about 15% to about 18%, about 30% to about 70%, about 35% to about 65%, about 63.13%, and about 40% to about 64% w/w. In exemplary formulations of the disclosure, the plasticizers will represent approximately 1 wt % to 75 wt %, preferably 2 wt % to 30 wt %, more preferably 5 wt. % to 20 wt. % of the formulation, more preferably in the range of 0.01%-95% w/w or w/v.

### Example 5

The continuous drug delivery systems and methods as disclosed herein may comprise emollients, humectants, skin irritation reducing agents and similar compounds or chemicals known to those skilled in the art either alone or in combinations thereof without any limitation to following like petrolatum, lanolin, mineral oil, dimethicone, zinc oxide, glycerin, propylene glycol and others. More preferably in the range of 0.01%-95% w/w or w/v. In exemplary embodiments, formulations of the disclosure may comprise emollients, humectants, skin irritation reducing agents and similar compounds at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 80%, about 95%, about 96%, about 97%, about 98%, and about 99% of the formulation. In exemplary embodiments, formulations of the disclosure may comprise emollients, humectants, skin irritation reducing agents and similar compounds at a concentration of about 1 to 20%, of about 5% to 25%, about 10% to about 20%, or about 15% to about 18%, about 30% to about 70%, about 35% to about 65%, about 63.13%, and about 40% to about 64% w/w. In exemplary formulations of the disclosure, the emollients, humectants, skin irritation reducing agents and similar compounds will represent approximately 1 wt % to 75 wt %, preferably 2 wt % to 30 wt %, more preferably 5 wt. % to 20 wt. % of the formulation, more preferably in the range of 0.01%-95% w/w or w/v.

### Example 6

The continuous drug delivery systems and methods as disclosed herein may comprise solubilizers, surfactants, emulsifying agents, dispersing agents and similar compounds or chemicals known to those skilled in the art either alone or in combination thereof without any limitation to following like polysorbate such as but not limited to (polysorbate 20, polysorbate 40, polysorbate 60, polysorbate 80 etc.), span such as but not limited to (span 80, span 20 etc.), surfactants such as (anionic, cationic, nonionic and amphoteric), propylene glycol monocaprylate type I, propylene glycol monocaprylate type II, propylene glycol dicaprylate, medium chain triglycerides, propylene glycol monolaurate type II, linoleoyl polyoxyl-6 glycerides, oleoyl-polyoxyl-6-glycerides, lauroyl polyoxyl-6-glycerides, polyglyceryl-3-dioleate, diethylene glycol monoethyl ether, propylene glycol monolaurate type I, polyglyceryl-3-dioleate, caprylocaproyl polyoxyl—8 glycerides etc, cyclodextrins and others. More preferably in the range of 0.01% 95% w/w or w/v. In exemplary embodiments, formulations of the disclosure may comprise solubilizers, surfactants, emulsifying agents, dispersing agents and similar compounds at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, about 80%, about 95%, about 96%, about 97%, about 98%, and about 99% of the formulation. In exemplary embodiments, formulations of the disclosure may comprise solubilizers, surfactants, emulsifying agents, dispersing agents and similar compounds at a concentration of about 1 to 20%, of about 5% to 25%, about 10% to about 20%, or about 15% to about 18%, about 30% to about 70%, about 35% to about 65%, about 63.13%, and about 40% to about 64% w/w. In exemplary formulations of the disclosure, the solubilizers, surfactants, emulsifying agents, dispersing agents and similar compounds will represent approximately 1 wt % to 75 wt %, preferably 2 wt % to 30 wt %, more preferably 5 wt. % to 20 wt. % of the formulation, more preferably in the range of 0.01% 95% w/w or w/v.

### Example 7

Different techniques and ingredients can be used to increase the stability and/or solubility of the active agents in formulation such as without any limitation to coating, encapsulation, microencapsulation, nanoencapsulation, lyophilization, chelating agents, complexing agents, packaging of a continuous drug delivery system, moisture scavengers, etc. Different stabilizers can be used to increase stability of active agents in formulation. etc.

### Example 8

The continuous drug delivery systems and methods as disclosed herein may comprise auxiliary pH buffering agents and pH stabilizers and similar compounds known to those skilled in the art which helps to maintain the appropriate pH of formulation preferably in the range of 4.0-8.0 either alone or in combination thereof without any limitation to following such as phosphate buffer, acetate buffer, citrate buffer, etc., acids such as but not limited to (carboxylic acids, inorganic acids, sulfonic acids, vinylogous carboxylic acids and others), base such as but not limited to (sodium hydroxide, potassium hydroxide, ammonium hydroxide, triethylamine, sodium carbonate, sodium bicarbonate) etc. More preferably in the range of 0.01%-30% w/w or w/v. In exemplary embodiments, formulations of the disclosure may comprise pH buffering agents and pH stabilizers and similar compounds at a concentration of about 0.01%, about 0.02%, about about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, about 80%, about 95%, about 96%, about 97%, about 98%, and about 99% of the formulation. In exemplary embodiments, formulations of the disclosure may comprise pH buffering agents and pH stabilizers and similar compounds at a concentration of about 1 to 20%, of about 5% to 25%, about 10% to about 20%, or about 15% to about 18%, about 30% to about 70%, about 35% to about 65%, about 63.13%, and about 40% to about 64% w/w. In exemplary formulations of the disclosure, the pH buffering agents and pH stabilizers and similar compounds will represent approximately 1 wt % to 75 wt %, preferably 2 wt % to 30 wt %, more preferably 5 wt. % to 20 wt. % of the formulation, more preferably in the range of 0.01%-30% w/w or w/v.

### Example 9

The continuous drug delivery systems and methods as disclosed herein may comprise antioxidants such as but not limited to (sodium metabisulfite, citric acid, ascorbic acid, BHA, BHT), oxidizing agents, stabilizers, discoloring agents, preservatives and similar compounds or chemicals known to those skilled in the art which helps to get a stable formulation can be used either alone or in combination thereof without any limitation. More preferably in the range of 0.01%-50% w/w or w/v. In exemplary embodiments, formulations of the disclosure may comprise antioxidants at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 80%, about 95%, about 96%, about 97%, about 98%, and about 99% of the formulation. In exemplary embodiments, formulations of the disclosure may comprise antioxidants at a concentration of about 1 to 20%, of about 5% to 25%, about 10% to about 20%, or about 15% to about 18%, about 30% to about 70%, about 35% to about 65%, about 63.13%, and about 40% to about 64% w/w. In exemplary formulations of the disclosure, the antioxidants will represent approximately 1 wt % to 75 wt %, preferably 2 wt % to 30 wt %, more preferably 5 wt. % to 20 wt. % of the formulation, more preferably in the range of 0.01%-50% w/w or w/v.

### Example 10

The continuous drug delivery systems and methods as disclosed herein may comprise drug formulated in ointment and/or cream base and/or gel and/or film forming transdermal formulation and/or transdermal matrix formulation and/or drug-in-adhesive matrix patch and/or matrix patch known to those skilled in the art.

### Example 11

Materials to make the continuous drug delivery systems and methods of the disclosure is known to those skilled in the art, for example, such as but not limited to a pump, reservoir patch, matrix patch, drug in adhesives, film forming formulation, micro-dosing transdermal patch, transdermal films and may include, such as but are not limited to polymers, copolymers, derivatives, backing film, release membranes, release liners, etc. either alone or in combinations thereof. Pressure sensitive adhesives (such as but not limited to silicone polymers, rubber based adhesives, acrylic polymers, acrylic copolymers, polyisobutylene, acrylic acid—isooctyl acrylate copolymer, hot melt adhesives, polybutylene etc.), backing film (such as but not limited to ethylene vinyl acetate copolymers, vinyl acetate resins, polyurethane, polyvinyl chloride, metal foils, polyester, aluminized films, polyethylene, etc.), release membrane (such as but not limited to microporous polyethylene membrane, microporous polypropylene membrane, rate controlling ethylene vinyl acetate copolymer membrane etc.), release liners (such as but not limited to siliconized polyester films, fluoropolymer coated polyester film, polyester film, siliconized polyethylene terephthalate film, etc.), tapes, etc.

The continuous drug delivery systems and methods as disclosed herein may deliver at least therapeutic effective dose of active agent, such as for example, CBD and/or THC, and derivatives of these compounds, alone or in combinations thereof in human plasma required for treating and/or preventing and/or controlling chronic pain. Therapeutically effective active agent such as CBD and/or THC, and derivatives of these compounds dosages refers to the therapeutic concentration of in human plasma required for treating and/or preventing and/or controlling chronic pain. Furthermore, the precise therapeutic effective dose of such as CBD and/or THC, and derivatives of these compounds in the continuous drug delivery system such as a pump, transdermal formulation or topical formulation or transdermal delivery system can be determined by those skilled in the art based on factors such as but not limited to the patient's condition etc. The transdermal formulation or topical formulation or transdermal delivery system will be available in different dosage strengths and patch sizes in order to achieve optimum therapeutic outcome based on patient's requirement.

The continuous drug delivery systems and methods such as a pump, or transdermal formulation or transdermal patch of active agents such as CBD and/or THC, and derivatives of these compounds can be applied to the skin surface in any of the following dosage regimens such as once in a day, once in two days, once in three days, once in four days, once in five days, once in six days, once in a week, once in a range of from about 8 to about 13 days, once in two weeks, or once in 15 days.

### Example 12

### Example Formulations

| Component | % W/W |
|---|---|
| Active component (THC and/or CBD) | 1%-30% |
| Solvent | 2%-30% |
| Permeation enhancer | 2%-30% |
| Pressure sensitive adhesive polymer | 20%-80% |
| Polymer | 1%-10% |

### Example Formulation

| Component | % W/W |
|---|---|
| Active component (THC and/or CBD) | 1%-30% |
| Solvent | 2%-30% |
| Permeation enhancer | 2%-30% |
| Pressure sensitive adhesive polymer | 20%-80% |

While the invention has been described in detail and with reference to specific examples thereof, it will be apparent to one skilled in the art that various changes and modifications can be made therein without departing from the spirit and scope thereof.

What is claimed is:

1. A method of treating, preventing, and/or slowing the onset or progression of a disease or condition and/or related symptom in a patient comprising:
selecting a patient in need of treating, preventing, and/or slowing the onset or progression of a disease or condition and/or a related symptom;
administering to the patient a pharmaceutical composition in a continuous delivery system, the pharmaceutical composition comprising at least one active agent selected from the group consisting of:
about 0.1% to about 50% of an active agent selected from the group consisting of cannabidiol (CBD), synthetic forms thereof, biosynthetic forms thereof, free base forms thereof, salts thereof, isomers thereof, amorphous forms thereof, derivatives thereof, and combinations thereof;
about 0.1% to about 50% of an active agent selected from the group consisting of tetrahydrocannabinol (THC), synthetic forms thereof, biosynthetic forms thereof, free base forms thereof, salts thereof, isomers thereof, amorphous forms thereof, derivatives thereof, and combinations thereof;
optionally, at least one additional active agent, and
combinations thereof
wherein the disease or condition and/or related symptom is treated, prevented, and/or slowed in the patient.

2. The method of claim 1 wherein the continuous delivery system provides a continuous, sustained delivery of the pharmaceutical composition to mitigate peak and valley pharmacokinetic behavior of the active agent.

3. The method of claim 1 wherein the continuous delivery system provides a continuous, sustained delivery of the pharmaceutical composition via administration to the patient by a route selected from the group consisting of parenteral, intravenous, subcutaneous, intramuscular, intrathecal, oral, buccal, mucosal, intranasal, rectal, vaginal, transdermal, implantable, topical, and combinations thereof.

4. The method of claim 1 wherein the continuous delivery system provides a continuous, sustained delivery of the pharmaceutical composition to the patient via intravenous or subcutaneous infusion.

5. The method of claim 1 wherein the continuous delivery system provides a continuous, sustained delivery of the pharmaceutical composition to the patient via subcutaneous infusion.

6. The method of claim 1 wherein the continuous delivery system provides a continuous, sustained delivery of the pharmaceutical composition to the patient via an infusion pump device.

7. The method of claim 1 wherein the continuous delivery system provides a continuous, sustained delivery of the pharmaceutical composition to the patient via an ambulatory/portable infusion pump that is worn by the patient and may use replaceable cartridges.

8. The method of claim 1 wherein the continuous delivery system provides a continuous, sustained delivery of the pharmaceutical composition to the patient via a patch pump or micro pump.

9. The method of claim 1 wherein the continuous delivery system comprises a pharmaceutical composition selected from the group consisting of a liquid formulation, a solid formulation, a semi-solid formulation, an emulsion formulation, a nanoparticle formulation, a matrix formulation, a film formulation, a patch formulation, a formulation in an infusion pump, and/or combinations thereof.

10. The method of claim 1 wherein the continuous delivery system comprises a pharmaceutical composition selected from the group consisting of transdermal liquid formulation, transdermal semisolid formulation, transdermal gel formulation, or transdermal polymer matrix formulation, transdermal adhesive matrix formulation, transdermal film forming gel, transdermal film forming spray formulation, multilayer transdermal matrix system, transdermal drug-in-adhesive matrix formulation, and combinations thereof.

11. The method of claim 1 wherein the continuous delivery system comprises a pharmaceutical composition formulated as a topical liquid formulation, topical semisolid formulation, topical gel formulation, topical polymer matrix formulation, topical adhesive matrix formulation, topical film forming gel formulation, or topical film forming spray formulation.

12. The method of claim 1 wherein the continuous delivery system comprises a pharmaceutical composition formulated as a transdermal patch, wherein the transdermal patch is selected from the group such as to reservoir patch, a multilayer transdermal matrix system, a microreservoir patch, a matrix patch, a drug in adhesive patch, a matrix patch of adhesive in combination with polymers, a pressure sensitive adhesive patch, extended-release transdermal film a liquid reservoir system, a microreservoir patch, a mucoadhesive patch, and combinations thereof.

13. The method of claim 1 wherein the continuous delivery system comprises a pharmaceutical composition formulated as a topical patch, wherein the topical patch is selected from the group such as a multilayer transdermal matrix system, reservoir patch, a microreservoir patch, a matrix patch, a drug in adhesive patch, a pressure sensitive adhesive patch, extended-release transdermal film a liquid reservoir system, a microreservoir patch, a mucoadhesive patch, a micro-dosing patch, a matrix patch of adhesive in combination with polymers, and combinations thereof.

14. The method of claim 1 wherein the continuous delivery system comprises a pharmaceutical composition formulated as a transdermal patch.

15. The method of claim 1 wherein the continuous delivery system comprises a pharmaceutical composition formulated as a multilayer transdermal matrix system, a metered dose transdermal gel, metered dose transdermal spray, a film forming gel, a film forming spray, or a meter-dose aerosol.

16. The method of claim 1 wherein the continuous delivery system comprises a pharmaceutical composition formulated as a topical patch.

17. The method of claim 1 wherein the continuous delivery system comprises a pharmaceutical composition formulated as metered dose gel, metered dose spray, gel, cream, solution, emulsion, liquid compositions, semisolid compositions, a matrix of adhesive in combination with polymers, or film forming formulation.

18. The method of claim 1 wherein the continuous delivery system comprises a pharmaceutical composition further comprising carriers or ingredients in effective amount selected from the group consisting of solvents, gelling agents, polymers, pressure sensitive adhesive polymers, penetration enhancers, emollients, skin irritation reducing agents, buffering agents, pH stabilizers, tackifier, diluent, bulking agent, solubilizers, suspending agents, dispersing agents, stabilizers, plasticizers, surfactants, antioxidants, oxidants, and combinations thereof.

19. The method of claim 1 wherein the continuous delivery system comprises a pharmaceutical composition further comprising carriers or ingredients in effective amount selected from the group consisting of solvents, gelling agents, polymers, pressure sensitive adhesive polymers, penetration enhancers, emollients, skin irritation reducing agents, buffering agents, pH stabilizers, solubilizers, suspending agents, dispersing agents, stabilizers, plasticizers, tackifiers, diluents, bulking agents, surfactants, antioxidants, oxidants, and combinations thereof in the range of 0.1%-99.5% w/w or w/v.

20. The method of claim 1 wherein the continuous delivery system comprises a pharmaceutical composition formulated as a transdermal formulation which can be administered in a dosage regimen selected from the group consisting of once daily, twice daily, three times a day, once in 1-8 hrs, once in 1-24 hrs, once in two days, once in three days, once in four days, once in five days, once in six days, once in a week, once in a 8 to about 13 days, once in two weeks, and once in 15 days to about 30 days.

21. The method of claim 1 wherein the continuous delivery system comprises a pharmaceutical composition formulated as a topical formulation which can be administered in a dosage regimen selected from the group consisting of once daily, twice daily, three times a day, four times a day, five times a day, six times a day, once in 1-8 hrs, once in 1-24 hrs, once in two days, once in three days, once in four days, once in five days, once in six days, once in a week, once in a 8 to about 13 days, once in two weeks, and once in 15 days to about 30 days.

22. The method of claim 1 wherein the continuous delivery system provides a continuous, sustained delivery of the pharmaceutical composition to the patient via microneedles.

23. The method of claim 1 wherein the continuous delivery system provides a blood serum level of active agent selected from the group consisting of about 0.01 ng/mL, about 0.02 ng/mL, about 0.05 ng/mL, about 0.1 ng/mL, about 0.2 ng/mL, about 0.5 ng/mL, about 1 ng/mL, about 2 ng/mL, about 5 ng/mL, about 10 ng/mL, about 20 ng/mL, about 50 ng/mL, about 100 ng/mL, about 200 ng/mL, about 500 ng/mL, about 1 μg/mL, about 2 μg/mL, and about 5 μg/mL.

24. The method of claim 1 wherein the continuous delivery system comprises a pharmaceutical composition wherein active agent is present at a concentration selected from the group consisting of about 1% to about 10%, about 1% to about 9%, about 1% to about 8%, about 1% to about 7%, about 1% to about 6%, about 1% to about 5%, about 0.1 to about 50%, about 1 to 20%, of about 5% to 25%, about 10% to about 20%, or about 15% to about 18%, about 30% to about 70%, and about 35% to about 65% of the formulation.

25. The method of claim 1 wherein the continuous delivery system comprises a pharmaceutical composition wherein active agent is present at a concentration selected from the group consisting of about 1% to about 10%, about 1% to about 9%, about 1% to about 8%, about 1% to about 7%, about 1% to about 6%, and about 1% to about 5% of the formulation.

26. The method of claim 1 wherein the continuous delivery system comprises a pharmaceutical composition wherein THC is selected from the group comprising of free base thereof, salts thereof, isomers thereof, amorphous forms thereof, crystalline forms thereof, co-crystalline forms thereof, prodrugs thereof, analogs thereof, derivatives thereof, synthetic forms thereof, biosynthetic forms thereof, naturally occurring forms thereof, active metabolites thereof, polymorph thereof, solid solution thereof, coated form thereof, stereoisomers thereof, solid solution thereof, ion-pair thereof, solution thereof, powder form thereof, liquid form thereof, alone or combinations thereof.

27. The method of claim 1 wherein the continuous delivery system comprises a pharmaceutical composition wherein CBD is selected from the group comprising of free base thereof, salts thereof, isomers thereof, amorphous forms thereof, crystalline forms thereof, co-crystalline forms thereof, prodrugs thereof, analogs thereof, derivatives thereof, synthetic forms thereof, biosynthetic forms thereof, naturally occurring forms thereof, active metabolites thereof, polymorph thereof, solid solution thereof, coated form thereof, ion-pairs thereof, stereoisomers thereof, solid solution thereof, solution thereof, powder form thereof, liquid form thereof, alone or combinations thereof.

28. The method of claim 1 wherein the continuous delivery system comprises a pharmaceutical composition which comprises one or more active agents selected from the group consisting of tetrahydrocannabinol (THC), cannabidiol (CBD), the free base thereof, salts thereof, isomers thereof, amorphous forms thereof, crystalline forms thereof, co-crystalline forms thereof, prodrugs thereof, analogs thereof, derivatives thereof, synthetic forms thereof, biosynthetic forms thereof, naturally occurring forms thereof, active metabolites thereof, polymorph thereof, solid solution thereof, coated form thereof, and combinations thereof, in a dosage form for transdermal delivery.

29. The method of claim 1 wherein the continuous delivery system comprises a pharmaceutical composition wherein said tetrahydrocannabinol (THC), cannabidiol (CBD), the free base thereof, salts thereof, isomers thereof, amorphous forms thereof, polymorphs forms thereof, stereoisomers thereof, ion-pairs thereof, coated forms thereof, crystalline forms thereof, co-crystalline forms thereof, prodrugs thereof, analogs thereof, derivatives thereof, synthetic forms thereof, biosynthetic forms thereof, naturally occurring forms thereof, active metabolites thereof, and combinations thereof is produced by a synthetic route.

30. The method of claim 1 wherein the continuous delivery system comprises a pharmaceutical composition wherein active agent is produced synthetically and has a purity equal to or greater than about 90%, 91%, 92%, 93%, 94%, 95%, 96%, 97%, 98%, 99%, 99.5%, 99.75%, or 100% (w/w) before being added to said pharmaceutical composition.

31. The method of claim 1 wherein the continuous delivery system comprises a pharmaceutical composition which is co-administered with at least one additional an active agent selected from the group consisting of: a dopamine precursor, a dopamine receptor agonist, and combinations thereof.

32. The method of claim 1 wherein the continuous delivery system comprises a pharmaceutical composition which is co-administered with at least one additional an active agent selected from the group consisting of: levodopa, bromocriptine, pergolide, pramipexole, cabergoline, ropinirole, apomorphine, and any combination thereof.

33. The method of claim 1 wherein the disease or condition and/or related symptom is selected from the group consisting of cancer, pain, seizure disorder, Parkinson's Disease (“PD”), and a symptom of PD.

34. The method of claim 33 wherein the disease or condition and/or related symptom is cancer which is selected from the group consisting of endometrial cancer, breast cancer, ovarian cancer, cervical cancer, fallopian tube cancer, testicular cancer, primary peritoneal cancer, colon cancer, colorectal cancer, small intestine cancer, squamous cell carcinoma of the anogenital region, melanoma, renal cell carcinoma, lung cancer, non-small cell lung cancer, squamous cell carcinoma of the lung, stomach cancer, bladder cancer, gall bladder cancer, liver cancer, thyroid cancer, laryngeal cancer, salivary gland cancer, esophageal cancer, head and neck cancer, squamous cell carcinoma of the head and neck, prostate cancer, pancreatic cancer, mesothelioma, Merkel cell carcinoma, sarcoma, glioblastoma, GBM, and a hematological cancer, such as multiple myeloma, B-cell lymphoma, T-cell lymphoma, Hodgkin's lymphoma/primary mediastinal B-cell lymphoma, and chronic myelogenous leukemia.

35. The method of claim 34 wherein the cancer is glioblastoma, GBM.

36. The method of claim 1 wherein the pharmaceutical composition is co-administered with at least one additional therapeutic selected from the group consisting of: temozolomide, peptides, polypeptides, fusion proteins, nucleic acid molecules, small molecules, mimetic agents, synthetic drugs, inorganic molecules, organic molecules, chemotherapies, radiation therapies, hormonal therapies, anti-angiogenesis therapies, targeted therapies, and/or biological therapies including immunotherapies and surgery, and combinations thereof.

37. The method of claim 33 wherein the disease or condition and/or related symptom is pain which is chronic pain selected from the group consisting of neuropathic pain, peripheral neuropathic pain, inflammatory pain, musculoskeletal pain, pain due to muscle spasms, pain due to increased muscle tone, osteoarthritic pain, muscular headache, tension-type headache, migraine, cluster headache, atypical facial pain, referred pain, vulvodynia, proctodynia, and any combination thereof.

38. The method of claim 33 wherein the disease or condition and/or related symptom is seizure disorder selected from the group consisting of complex partial seizures, simple partial seizures, partial seizures with secondary generalization, generalized seizures (including absence, grand mal (tonic clonic), status epilepticus, tonic, atonic, myoclonic), neonatal and infantile spasms, drug-induced seizures, trauma-induced seizures, and febrile seizures, and additional specific epilepsy syndromes such as juvenile myoclonic epilepsy, Lennox-Gastaut, Dravet syndrome, Tuberous Sclerosis Complex (TSC), Treatment-Resistant Epilepsy, Treatment Resistant Pediatric Epilepsy, mesial temporal lobe epilepsy, nocturnal frontal lobe epilepsy, progressive epilepsy with mental retardation, and progressive myoclonic epilepsy, and seizures associated with CNS mass lesions.

39. The method of claim 38 wherein the seizure disorder is selected from the group consisting of complex partial seizures, simple partial seizures, partial seizures with secondary generalization, generalized seizures (including absence, grand mal (tonic clonic), status epilepticus, tonic, atonic, myoclonic), neonatal and infantile spasms, drug-induced seizures, trauma-induced seizures, and febrile seizures, and additional specific epilepsy syndromes such as juvenile myoclonic epilepsy, Lennox-Gastaut, Dravet syndrome, Tuberous Sclerosis Complex (TSC), Treatment-Resistant Epilepsy, Treatment Resistant Pediatric Epilepsy, mesial temporal lobe epilepsy, nocturnal frontal lobe epilepsy, progressive epilepsy with mental retardation, and progressive myoclonic epilepsy, as well as seizures associated with CNS mass lesions.

40. The method of claim 33 wherein the disease or condition and/or related symptom is a symptom of PD to be treated, prevented, and/or slowed and is selected from the group consisting of:
(a) at least one non-motor aspect of experiences of daily living as defined by Part I of the Unified Parkinson's Disease Rating Scale selected from the group consisting of cognitive impairment, hallucinations and psychosis, depressed mood, anxious mood, apathy, features of dopamine dysregulation syndrome, sleep problems, daytime sleepiness, pain, urinary problems, constipation problems, lightheadedness on standing, and fatigue;
(b) at least one motor aspect of experiences of daily living as defined by Part II of the Unified Parkinson's Disease Rating Scale selected from the group consisting of speech, saliva and drooling, chewing and swallowing, eating tasks, dressing, hygiene, handwriting, turning in bed, tremors, getting out of a bed, a car, or a deep chair, walking and balance, and freezing;
(c) at least one motor symptom identified in Part III of the Unified Parkinson's Disease Rating Scale selected from the group consisting of speech, facial expression, rigidity, finger tapping, hand movements, pronation-supination movements of hands, toe tapping, leg agility, arising from chair, gait, freezing of gait, postural stability, posture, body bradykinesia, postural tremor of the hands, kinetic tremor of the hands, rest tremor amplitude, and constancy of rest tremor;
(d) at least one motor complication identified in Part IV of the Unified Parkinson's Disease Rating Scale selected from the group consisting of time spent with dyskinesias, functional impact of dyskinesias, time spent in the off state, functional impact of fluctuations, complexity of motor fluctuations, and painful off-state dystonia;
(e) constipation;
(f) depression;
(g) cognitive impairment;
(h) short or long term memory impairment;
(i) concentration impairment;
(j) coordination impairment;
(k) mobility impairment;
(l) speech impairment;
(m) mental confusion;
(n) sleep problem, sleep disorder, or sleep disturbance;
(o) circadian rhythm dysfunction;
(p) hallucinations;
(q) fatigue;
(r) REM disturbed sleep;
(s) REM behavior disorder;
(t) erectile dysfunction;
(u) postural hypotension;
(v) correction of blood pressure or orthostatic hypotension;
(w) nocturnal hypertension;
(x) regulation of temperature;
(y) improvement in breathing or apnea;
(z) correction of cardiac conduction defect;
(aa) amelioration of pain;
(bb) urinary incontinence, or restoration of bladder sensation and urination;
(cc) mood swings;
(dd) apathy;
(ee) control of nocturia;
(ff) neurodegeneration;
(gg) anxiety;
(hh) improving speaking ability, including public speaking ability; and/or
(ii) Parkinson's Disease Dementia.

41. The method of claim 33 wherein the disease or condition and/or related symptom is a PD symptom to be treated, prevented, and/or slowed which is a sleep problem, sleep disorder, sleep disturbance, circadian rhythm dysfunction, REM disturbed sleep, or REM behavior disorder, and wherein:
(a) the sleep disorder or sleep disturbance comprises a delay in sleep onset, sleep fragmentation, REM-behavior disorder, sleep-disordered breathing including snoring and apnea, day-time sleepiness, micro-sleep episodes, narcolepsy, hallucinations, or any combination thereof; and/or
(b) the REM-behavior disorder comprises vivid dreams, nightmares, and acting out the dreams by speaking or screaming, or fidgeting or thrashing of arms or legs during sleep; and/or
(c) treating the sleep problem, sleep disorder, sleep disturbance prevents or delays the onset and/or progression of the Parkinson's disease; and/or
(d) the method results in a positive change in the sleeping pattern of the subject over a defined period of time; and/or
(e) the method results in a positive change in the sleeping pattern of the subject over a defined period of time, wherein the positive change is defined as:
(i) an increase in the total amount of sleep obtained of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or
(ii) a percent decrease in the number of awakenings during the night selected from the group consisting of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; and/or
(f) as a result of the method the subject obtains the total number of hours of sleep recommended by a medical authority for the age group of the subject; and/or
(g) wherein each defined period of time is independently selected from the group consisting of about 1 day to about 10 days, about 10 days to about 30 days, about 30 days to about 3 months, about 3 months to about 6 months, about 6 months to about 12 months, and about greater than 12 months.

42. The method of claim 33 wherein the disease or condition and/or related symptom is a PD symptom to be treated, prevented, and/or slowed which is hallucination and wherein:
(a) the hallucination comprises a visual, auditory, tactile, gustatory or olfactory hallucination; and/or
(b) treating the hallucination prevents and/or delays the onset and/or progression of the Parkinson's disease; and/or
(c) the method results in a decreased number of hallucinations of the subject over a defined period of time; and/or
(d) the method results in a decreased number of hallucinations of the subject over a defined period of time and the decrease in number is selected from the group consisting of by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or
(e) the method results in the subject being hallucination-free; and/or
(f) the method results in a decreased severity of hallucinations of the subject over a defined period of time, as measured by one or more medically recognized technique; and/or
(g) the method results in a decreased severity of hallucinations of the subject over a defined period of time and the decrease in severity is selected from the group consisting of by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%, as measured by one or more medically recognized technique; and/or
(h) the medically recognized technique selected from the group consisting of Chicago Hallucination Assessment Tool (CHAT), The Psychotic Symptom Rating Scales (PSYRATS), Auditory Hallucinations Rating Scale (AHRS), Hamilton Program for Schizophrenia Voices Questionnaire (HPSVQ), Characteristics of Auditory Hallucinations Questionnaire (CAHQ), Mental Health Research Institute Unusual Perception Schedule (MUPS), positive and negative syndrome scale (PANSS), scale for the assessment of positive symptoms (SAPS), Launay-Slade hallucinations scale (LSHS), the Cardiff anomalous perceptions scale (CAPS), and structured interview for assessing perceptual anomalies (SIAPA); and/or
(i) each defined period of time is independently selected from the group consisting of about 1 day to about 10 days, about 10 days to about 30 days, about 30 days to about 3 months, about 3 months to about 6 months, about 6 months to about 12 months, and about greater than 12 months.

43. The method of claim 33 wherein the disease or condition and/or related symptom is a PD symptom to be treated, prevented, and/or slowed which is depression and wherein:
(a) treating the depression prevents and/or delays the onset and/or progression of the Parkinson's disease; and/or
(b) the method results in improvement in a subject's depression over a defined period of time, as measured by one or more clinically-recognized depression rating scale; and/or
(c) the method results in improvement in a subject's depression over a defined period of time, as measured by one or more clinically-recognized depression rating scale and the improvement is in one or more depression characteristics selected from the group consisting of mood, behavior, bodily functions such as eating, sleeping, energy, and sexual activity, and/or episodes of sadness or apathy; and/or
(d) the method results in improvement in a subject's depression, as measured by one or more clinically-recognized depression rating scale, and the improvement a subject experiences following treatment is about 5, about 10, about 15, about 20, about 25, about 30, about 35, about 40, about 45, about 50, about 55, about 60, about 65, about 70, about 75, about 80, about 85, about 90, about 95 or about 100%; and/or
(e) wherein each defined period of time is independently selected from the group consisting of about 1 day to about 10 days, about 10 days to about 30 days, about 30 days to about 3 months, about 3 months to about 6 months, about 6 months to about 12 months, and about greater than 12 months.

44. The method of claim 33 wherein the disease or condition and/or related symptom is a PD symptom to be treated, prevented, and/or slowed is cognitive impairment, and wherein:
(a) treating the cognitive impairment prevents and/or delays the onset and/or progression of the Parkinson's disease; and/or
(b) progression or onset of the cognitive impairment is slowed, halted, or reversed over a defined period of time following administration of the pharmaceutical composition, as measured by a medically-recognized technique; and/or
(c) the cognitive impairment is positively impacted by the administration of the pharmaceutical composition, as measured by a medically-recognized technique; and/or
(d) the cognitive impairment is positively impacted by the administration of the pharmaceutical composition, as measured by a medically-recognized technique and the positive impact on and/or progression of cognitive decline is measured quantitatively or qualitatively by one or more techniques selected from the group consisting of Mini-Mental State Exam (MMSE), Mini-cog test, and a computerized tested selected from Cantab Mobile, Cognigram, Cognivue, Cognision, or Automated Neuropsychological Assessment Metrics; and/or
(e) the progression or onset of cognitive impairment is slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique; and/or
(f) each defined period of time is independently selected from the group consisting of about 1 day to about 10 days, about 10 days to about 30 days, about 30 days to about 3 months, about 3 months to about 6 months, about 6 months to about 12 months, and about greater than 12 months.

45. The method of claim 33 wherein the disease or condition and/or related symptom is PD symptom to be treated, prevented, and/or slowed which is constipation, and wherein:
(a) treating the constipation prevents and/or delays the onset and/or progression of the Parkinson's disease; and/or
(b) the administration of the pharmaceutical composition causes the subject to have a bowel movement; and/or
(c) the method results in an increase in the frequency of bowel movement in the subject over a defined period of time; and/or
(d) the method results in an increase in the frequency of bowel movement in the subject and the increase in the frequency of bowel movement is defined as:
(i) an increase in the number of bowel movements per week of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or
(ii) a percent decrease in the amount of time between each successive bowel movement selected from the group consisting of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; and/or
(e) as a result of the method the subject has the frequency of bowel movement recommended by a medical authority for the age group of the subject.

46. The method of claim 33 wherein the disease or condition and/or related symptom is a PD symptom to be treated, prevented, and/or slowed which is neurodegeneration correlated with PD, and wherein:
(a) treating the neurodegeneration prevents and/or delays the onset and/or progression of the Parkinson's disease; and/or
(b) the method results in treating, preventing, and/or delaying the progression and/or onset of neurodegeneration in the subject; and/or
(c) progression or onset of the neurodegeneration is slowed, halted, or reversed over a defined period of time following administration of the pharmaceutical composition, as measured by a medically-recognized technique; and/or
(d) the neurodegeneration is positively impacted by the administration of the pharmaceutical composition, as measured by a medically-recognized technique; and/or
(e) the positive impact and/or progression of neurodegeneration is measured quantitatively or qualitatively by one or more techniques selected from the group consisting of electroencephalogram (EEG), neuroimaging, functional Mill, structural Mill, diffusion tensor imaging (DTI), [18F]fluorodeoxyglucose (FDG) PET, agents that label amyloid, [18F]F-dopa PET, radiotracer imaging, volumetric analysis of regional tissue loss, specific imaging markers of abnormal protein deposition, multimodal imaging, and biomarker analysis; and/or
(f) the progression or onset of neurodegeneration is slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique; and/or
(g) each defined period of time is independently selected from the group consisting of about 1 day to about 10 days, about 10 days to about 30 days, about 30 days to about 3 months, about 3 months to about 6 months, about 6 months to about 12 months, and about greater than 12 months.

47. A continuous delivery system comprising a pharmaceutical composition comprising at least one active agent selected from the group consisting of:
about 0.1% to about 50% of an active agent selected from the group consisting of cannabidiol (CBD), synthetic forms thereof, biosynthetic forms thereof, free base forms thereof, salts thereof, isomers thereof, amorphous forms thereof, derivatives thereof, and combinations thereof;
about 0.1% to about 50% of an active agent selected from the group consisting of tetrahydrocannabinol (THC), synthetic forms thereof, biosynthetic forms thereof, free base forms thereof, salts thereof, isomers thereof, amorphous forms thereof, derivatives thereof, and combinations thereof;
optionally, at least one additional active agent, and
combinations thereof.

48. The continuous delivery system of claim 47 wherein the continuous delivery system provides a continuous, sustained delivery of the pharmaceutical composition to mitigate peak and valley pharmacokinetic behavior of the active agent.

49. The continuous delivery system claim 47 wherein the continuous delivery system provides a continuous, sustained delivery of the pharmaceutical composition via administration to the patient by a route selected from the group consisting of parenteral, intravenous, subcutaneous, intramuscular, intrathecal, oral, buccal, mucosal, intranasal, rectal, vaginal, transdermal, implantable, topical, and combinations thereof.

50. The continuous delivery system of claim 47 wherein the continuous delivery system provides a continuous, sustained delivery of the pharmaceutical composition via intravenous or subcutaneous infusion.

51. The continuous delivery system of claim 47 wherein the continuous delivery system provides a continuous, sustained delivery of the pharmaceutical composition via subcutaneous infusion.

52. The continuous delivery system of claim 47 wherein the continuous delivery system provides a continuous, sustained delivery of the pharmaceutical composition via an infusion pump device.

53. The continuous delivery system of claim 47 wherein the continuous delivery system provides a continuous, sustained delivery of the pharmaceutical composition via an ambulatory/portable infusion pump that is worn by the patient and may use replaceable cartridges

54. The continuous delivery system of claim 47 wherein the continuous delivery system provides a continuous, sustained delivery of the pharmaceutical composition via a patch pump or micro pump.

55. The continuous delivery system of claim 47 wherein the continuous delivery system comprises a pharmaceutical composition selected from the group consisting of a liquid formulation, a solid formulation, a semi-solid formulation, an emulsion formulation, a nanoparticle formulation, a matrix formulation, a film formulation, a patch formulation, a formulation in an infusion pump, and/or combinations thereof.

56. The continuous delivery system of claim 47 wherein the continuous delivery system comprises a pharmaceutical composition selected from the group consisting of transdermal liquid formulation, transdermal semisolid formulation, transdermal gel formulation, or transdermal polymer matrix formulation, transdermal adhesive matrix formulation, transdermal film forming gel, transdermal film forming spray formulation, multilayer transdermal matrix system, transdermal drug-in-adhesive matrix formulation, and combinations thereof.

57. The continuous delivery system of claim 47 wherein the continuous delivery system comprises a pharmaceutical composition formulated as a topical liquid formulation, topical semi solid formulation, topical gel formulation, topical polymer matrix formulation, topical adhesive matrix formulation, topical film forming gel formulation, or topical film forming spray formulation.

58. The continuous delivery system of claim 47 wherein the continuous delivery system comprises a pharmaceutical composition formulated as a transdermal patch, wherein the transdermal patch is selected from the group such as to reservoir patch, a multilayer transdermal matrix system, a microreservoir patch, a matrix patch, a drug in adhesive patch, a matrix patch of adhesive in combination with polymers, a pressure sensitive adhesive patch, extended-release transdermal film a liquid reservoir system, a microreservoir patch, a mucoadhesive patch, and combinations thereof.

59. The continuous delivery system of claim 47 wherein the continuous delivery system comprises a pharmaceutical composition formulated as a topical patch, wherein the topical patch is selected from the group such as a multilayer transdermal matrix system, reservoir patch, a microreservoir patch, a matrix patch, a drug in adhesive patch, a pressure sensitive adhesive patch, extended-release transdermal film a liquid reservoir system, a microreservoir patch, a mucoadhesive patch, a micro-dosing patch, a matrix patch of adhesive in combination with polymers, and combinations thereof.

60. The continuous delivery system of claim 47 wherein the continuous delivery system comprises a pharmaceutical composition formulated as a transdermal patch.

61. The continuous delivery system of claim 47 wherein the continuous delivery system comprises a pharmaceutical composition formulated as a multilayer transdermal matrix system, a metered dose transdermal gel, metered dose transdermal spray, a film forming gel, a film forming spray, or a meter-dose aerosol.

62. The continuous delivery system of claim 47 wherein the continuous delivery system comprises a pharmaceutical composition formulated as a topical patch.

63. The continuous delivery system of claim 47 wherein the continuous delivery system comprises a pharmaceutical composition formulated as metered dose gel, metered dose spray, gel, cream, solution, emulsion, liquid compositions, semisolid compositions, a matrix of adhesive in combination with polymers, or film forming formulation.

64. The continuous delivery system of claim 47 wherein the continuous delivery system comprises a pharmaceutical composition further comprising carriers or ingredients in effective amount selected from the group consisting of solvents, gelling agents, polymers, pressure sensitive adhesive polymers, penetration enhancers, emollients, skin irritation reducing agents, buffering agents, pH stabilizers, tackifier, diluent, bulking agent, solubilizers, suspending agents, dispersing agents, stabilizers, plasticizers, surfactants, antioxidants, oxidants, and combinations thereof.

65. The continuous delivery system of claim 47 wherein the continuous delivery system comprises a pharmaceutical composition further comprising carriers or ingredients in effective amount selected from the group consisting of solvents, gelling agents, polymers, pressure sensitive adhesive polymers, penetration enhancers, emollients, skin irritation reducing agents, buffering agents, pH stabilizers, solubilizers, suspending agents, dispersing agents, stabilizers, plasticizers, tackifiers, diluents, bulking agents, surfactants, antioxidants, oxidants, and combinations thereof in the range of 0.1%-99.5% w/w or w/v.

66. The continuous delivery system of claim 47 wherein the continuous delivery system comprises a pharmaceutical composition formulated as a transdermal formulation which can be administered in a dosage regimen selected from the group consisting of once daily, twice daily, three times a day, once in 1-8 hrs, once in 1-24 hrs, once in two days, once in three days, once in four days, once in five days, once in six days, once in a week, once in a 8 to about 13 days, once in two weeks, and once in 15 days to about 30 days.

67. The continuous delivery system of claim 47 wherein the continuous delivery system comprises a pharmaceutical composition formulated as a topical formulation which can be administered in a dosage regimen selected from the group consisting of once daily, twice daily, three times a day, four times a day, five times a day, six times a day, once in 1-8 hrs, once in 1-24 hrs, once in two days, once in three days, once in four days, once in five days, once in six days, once in a week, once in a 8 to about 13 days, once in two weeks, and once in 15 days to about 30 days.

68. The continuous delivery system of claim 47 wherein the continuous delivery system comprises a pharmaceutical composition which is formulated as microneedles.

69. The continuous delivery system of claim 47 wherein the continuous delivery system provides a blood serum level of active agent selected from the group consisting of about 0.01 ng/mL, about 0.02 ng/mL, about 0.05 ng/mL, about 0.1 ng/mL, about 0.2 ng/mL, about 0.5 ng/mL, about 1 ng/mL, about 2 ng/mL, about 5 ng/mL, about 10 ng/mL, about 20 ng/mL, about 50 ng/mL, about 100 ng/mL, about 200 ng/mL, about 500 ng/mL, about 1 μg/mL, about 2 μg/mL, and about 5 μg/mL.

70. The continuous delivery system of claim 47 wherein the continuous delivery system comprises a pharmaceutical composition wherein active agent is present at a concentration selected from the group consisting of about 1% to about 10%, about 1% to about 9%, about 1% to about 8%, about 1% to about 7%, about 1% to about 6%, about 1% to about 5%, about 0.1 to about 50%, about 1 to 20%, of about 5% to 25%, about 10% to about 20%, or about 15% to about 18%, about 30% to about 70%, and about 35% to about 65% of the formulation.

71. The continuous delivery system of claim 47 wherein the continuous delivery system comprises a pharmaceutical composition wherein active agent is present at a concentration selected from the group consisting of about 1% to about 10%, about 1% to about 9%, about 1% to about 8%, about 1% to about 7%, about 1% to about 6%, and about 1% to about 5% of the formulation.

72. The continuous delivery system of claim 47 wherein the continuous delivery system comprises a pharmaceutical composition wherein THC is selected from the group comprising of free base thereof, salts thereof, isomers thereof, amorphous forms thereof, crystalline forms thereof, co-crystalline forms thereof, prodrugs thereof, analogs thereof, derivatives thereof, synthetic forms thereof, biosynthetic forms thereof, naturally occurring forms thereof, active metabolites thereof, polymorph thereof, solid solution thereof, coated form thereof, stereoisomers thereof, solid solution thereof, ion-pair thereof, solution thereof, powder form thereof, liquid form thereof, alone or combinations thereof.

73. The continuous delivery system of claim 47 wherein the continuous delivery system comprises a pharmaceutical composition wherein CBD is selected from the group comprising of free base thereof, salts thereof, isomers thereof, amorphous forms thereof, crystalline forms thereof, co-crystalline forms thereof, prodrugs thereof, analogs thereof, derivatives thereof, synthetic forms thereof, biosynthetic forms thereof, naturally occurring forms thereof, active metabolites thereof, polymorph thereof, solid solution thereof, coated form thereof, ion-pairs thereof, stereoisomers thereof, solid solution thereof, solution thereof, powder form thereof, liquid form thereof, alone or combinations thereof.

74. The continuous delivery system of claim 47 wherein the continuous delivery system comprises a pharmaceutical composition which comprises one or more active agents selected from the group consisting of tetrahydrocannabinol (THC), cannabidiol (CBD), the free base thereof, salts thereof, isomers thereof, amorphous forms thereof, crystalline forms thereof, co-crystalline forms thereof, prodrugs thereof, analogs thereof, derivatives thereof, synthetic forms thereof, biosynthetic forms thereof, naturally occurring forms thereof, active metabolites thereof, polymorph thereof, solid solution thereof, coated form thereof, and combinations thereof, in a dosage form for transdermal delivery.

75. The continuous delivery system of claim 47 wherein the continuous delivery system comprises a pharmaceutical composition wherein said tetrahydrocannabinol (THC), cannabidiol (CBD), the free base thereof, salts thereof, isomers thereof, amorphous forms thereof, polymorphs forms thereof, stereoisomers thereof, ion-pairs thereof, coated forms thereof, crystalline forms thereof, co-crystalline forms thereof, prodrugs thereof, analogs thereof, derivatives thereof, synthetic forms thereof, biosynthetic forms thereof, naturally occurring forms thereof, active metabolites thereof, and combinations thereof is produced by a synthetic route.

76. The continuous delivery system of claim 47 wherein the continuous delivery system comprises a pharmaceutical composition wherein active agent is produced synthetically and has a purity equal to or greater than about 90%, 91%, 92%, 93%, 94%, 95%, 96%, 97%, 98%, 99%, 99.5%, 99.75%, or 100% (w/w) before being added to said pharmaceutical composition.

77. The continuous delivery system of claim 47 wherein the continuous delivery system comprises a pharmaceutical composition which is co-administered with at least one additional an active agent selected from the group consisting of: a dopamine precursor, a dopamine receptor agonist, and combinations thereof.

78. The continuous delivery system of claim 47 wherein the continuous delivery system comprises a pharmaceutical composition which is co-administered with at least one additional an active agent selected from the group consisting of: levodopa, bromocriptine, pergolide, pramipexole, cabergoline, ropinirole, apomorphine, and any combination thereof.
